(19) United States
(12) Patent Application Publication
(10) Pub. No.: US 2023/0151109 A1
(43) Pub. Date: May 18, 2023

(54) BISPECIFIC ANTIBODIES AGAINST CD9

(71) Applicant: UCB BIOPHARMA SRL, Brussels (BE)

(72) Inventors: David Alan COOK, Slough, Berkshire (GB); Helen Margaret FINNEY, Slough, Berkshire (GB); Stephen Edward RAPECKI, Slough, Berkshire (GB)

(21) Appl. No.: 17/929,031

(22) PCT Filed: Feb. 13, 2020
(86) PCT No.: PCT/EP2020/053780

§ 371 Date: Aug. 10, 2022

(51) Int. Cl. C07K 16/28; A61P 35/00

(52) U.S. Cl. CPC C07K 16/2896; C07K 16/2878; A61P 35/00; C07K 2317/31; C07K 2317/55; C07K 2317/622; C07K 2317/75

(57) ABSTRACT

The present invention relates to multispecific antibodies against a novel targets' combination of CD9 and another antigen, and their use in the treatment of cancer and infectious diseases.

### FIELD OF THE INVENTION

The present invention belongs to the field of multispecific antibodies binding at least CD9 and at least another antigen, and their uses in the treatment of cancer and/or infectious diseases.

### BACKGROUND OF THE INVENTION

T cells are key to a successful cell-mediated immune response necessary to eliminate cancer cells, bacteria and viruses. They recognise antigens displayed on the surface of tumour cells or antigens from bacteria and viruses replicating within the cells or from pathogens or pathogen products endocytosed from the extracellular fluid. T cells have two major roles. They can become cytotoxic T cells capable of destroying cells marked as foreign. Cytotoxic T cells have a unique surface protein called CD8, thus they are often referred to as CD8+ T cells. Alternatively, T cells can become helper T cells, which work to regulate and coordinate the immune system. Helper T cells have a unique surface protein called CD4 and are thus often called CD4+ T cells. Helper T cells have several important roles in the immune system: 1) responding to activation by specific antigens by rapidly proliferating; 2) signaling B cells to produce antibodies; and 3) activating macrophages.

Cancer eludes the immune system by exploiting mechanisms developed to avoid auto-immunity. However, the immune system is programmed to avoid immune over-activation which could harm healthy tissue. T cell activation is at the core of these mechanisms. Antigen specific T cells normally able to fight disease can become functionally tolerant (exhausted) to infectious agents or tumour cells by over stimulation or exposure to suppressive molecules. Therefore, molecules that enhance the natural function of T cells or overcome suppression of T cells have great utility in the treatment or prevention of cancer and infectious disease.

In recent years, immunotherapy has become an established treatment option for an increasing number of cancer patients, exemplified by the increased use of therapeutic antibody-based immune checkpoint inhibitors (CPI's). This has arisen from an increased immunological understanding of how cancer cells perturb immune cell activation by hijacking pathways normally involved in maintaining tolerance and skewing the balance between co-stimulation and co-inhibition (Chen and Mellman., Immunity. 39:1-105 (2013)). Amongst the pathways that have emerged as key regulators in this regard, include CTLA-4 and the PD-1/PD-L1 checkpoint molecules serving to down-regulate T cell and myeloid cell activation in the tumour microenvironment. Ipilimumab (anti-CTLA-4) was the first CPI to be approved in 2011 as a treatment for melanoma, closely followed by FDA approval of anti-PD1 directed antibodies, pembrolizumab and nivolumab in 2014 (Hargadon et al., International Immunopharmacol. 62:29-39 (2018)). There are still significant challenges in understanding differences in efficacy across patient groups, ranging from complete responses, to treatment relapse and even failure to respond, (Haslam and Prasad. JAMA Network Open.5:2e192535 (2019)). Despite the promising anti-tumour efficacy of several monoclonal antibodies, many cancers are refractory to treatments with a single antibody. Combinations of two or more antibodies are currently being tested in patients to provide improved methods of treatment. To date, these therapies rely on rational design of known mechanisms of action and are largely based on combining antigen-specificities known to be independently effective in the treatment of cancer, either as combination therapies or in a bispecific antibody format. This state of the art approach is a limiting factor in the development of new therapies as it relies on known therapies.

Therefore, there is still the need to identify novel modulators of T-cell activation based on unbiased biology, which allows to identify novel target pairs capable of enhancing T cell activation and induction of T cell proliferation for the treatment of cancer and infectious diseases.

### SUMMARY OF THE INVENTION

The present invention addresses the above-identified need by providing in a first aspect an antibody which comprises a first antigen-binding portion binding CD9 and a second antigen-binding portion binding another antigen.

In one embodiment, the second antigen-binding portion binds an antigen expressed on a cell surface, such as a cell of the immune system. The cell is preferably a T cell, a B cell or a NK cell.

In another embodiment, the second antigen-binding portion binds CD137, or HVEM, or CD7.

In one embodiment of this first aspect, each of the antigen-binding portions is independently a monoclonal antigen-binding portion.

In another embodiment, each of the antigen-binding portions is independently selected from a Fab, a Fab′, a scFv or a VHH. In yet another aspect, the antigen-binding portions are the antigen-binding portions of an IgG.

In another embodiment, the antibody which comprises a first antigen-binding portion binding CD9 and a second antigen-binding portion binding another antigen is chimeric, human or humanised, and preferably the antibody is humanised.

In another embodiment, the antibody comprises a heavy chain constant region selected from an IgG1, an IgG2, an IgG3 or an IgG4 isotype, or a variant thereof.

In another embodiment, the antibody further comprises at least an additional antigen-binding portion. The additional antigen-binding portion may be capable of increasing the half-life of the antibody. Preferably the additional antigen-binding portion binds albumin, more preferably human serum albumin.

In one embodiment, the first antigen binding portion binds CD9 in loop2, wherein preferably the first antigen-binding portion binds within amino acids 112 to 195 of SEQ ID NO:1.

In one embodiment, the first antigen-binding portion binding CD9 of the antibody of the present invention comprises a first heavy chain variable region and a first light chain variable region and the second antigen-binding portion comprises a second heavy chain variable region and a second light chain variable region and binds CD137 and wherein:
a. The first heavy chain variable region comprises a CDR-H1 comprising SEQ ID NO: 11, a CDR-H2 comprising SEQ ID NO: 12 and a CDR-H3 comprising SEQ ID NO: 13; and
b. The first light chain variable region comprises a CDR-L1 comprising SEQ ID NO: 14, a CDR-L2 comprising SEQ ID NO: 15 and a CDR-L3 comprising SEQ ID NO: 16; and
c. The second heavy chain variable region comprises a CDR-H1 comprising SEQ ID NO: 5, a CDR-H2 comprising SEQ ID NO: 6 and a CDR-H3 comprising SEQ ID NO: 7; and
d. The second light chain variable region comprises a CDR-L1 comprising SEQ ID NO: 8, a CDR-L2 comprising SEQ ID NO: 9 and a CDR-L3 comprising SEQ ID NO: 10;
or
e. The first heavy chain variable region comprises SEQ ID NO: 21 and the first light chain variable region comprises SEQ ID NO: 23; and the second heavy chain variable region comprises SEQ ID NO: 17 and second light chain variable region comprises SEQ ID NO: 19;
or
f. The first heavy chain variable region is encoded by a nucleotide sequence comprising SEQ ID NO: 22 and the first light chain variable region is encoded by a nucleotide sequence comprising SEQ ID NO: 24; and the second heavy chain variable region is encoded by a nucleotide sequence comprising SEQ ID NO: 18 and second light chain variable region is encoded by a nucleotide sequence comprising SEQ ID NO: 20.

In one embodiment, the first antigen-binding portion binding CD9 of the antibody of the present invention comprises a first heavy chain variable region and a first light chain variable region and the second antigen-binding portion comprises a second heavy chain variable region and a second light chain variable region and binds HVEM and wherein:
g. The first heavy chain variable region comprises a CDR-H1 comprising SEQ ID NO: 11, a CDR-H2 comprising SEQ ID NO: 12 and a CDR-H3 comprising SEQ ID NO: 13; and
h. The first light chain variable region comprises a CDR-L1 comprising SEQ ID NO: 14, a CDR-L2 comprising SEQ ID NO: 15 and a CDR-L3 comprising SEQ ID NO: 16; and
i. The second heavy chain variable region comprises a CDR-H1 comprising SEQ ID NO: 25, a CDR-H2 comprising SEQ ID NO: 26 and a CDR-H3 comprising SEQ ID NO: 27; and
j. The second light chain variable region comprises a CDR-LI comprising SEQ ID NO: 30;
or
k. The first heavy chain variable region comprises SEQ ID NO: 21 and the first light chain variable region comprises SEQ ID NO: 23; and the second heavy chain variable region comprises SEQ ID NO: 31 and second light chain variable region comprises SEQ ID NO: 33;
or
l. The first heavy chain variable region is encoded by a nucleotide sequence comprising SEQ ID NO: 22 and the first light chain variable region is encoded by a nucleotide sequence comprising SEQ ID NO: 24; and the second heavy chain variable region is encoded by a nucleotide sequence comprising SEQ ID NO: 32 and second light chain variable region is encoded by a nucleotide sequence comprising SEQ ID NO: 34.

In one embodiment, the first antigen-binding portion binding CD9 of the antibody of the present invention comprises a first heavy chain variable region and a first light chain variable region and the second antigen-binding portion comprises a second heavy chain variable region and a second light chain variable region and binds CD7 and wherein:
m. The first heavy chain variable region comprises a CDR-H1 comprising SEQ ID NO: 11, a CDR-H2 comprising SEQ ID NO: 12 and a CDR-H3 comprising SEQ ID NO: 13; and
n. The first light chain variable region comprises a CDR-L1 comprising SEQ ID NO: 14, a CDR-L2 comprising SEQ ID NO: 15 and a CDR-L3 comprising SEQ ID NO: 16; and
o. The second heavy chain variable region comprises a CDR-H1 comprising SEQ ID NO: 35, a CDR-H2 comprising SEQ ID NO: 36 and a CDR-H3 comprising SEQ ID NO: 37; and
p. The second light chain variable region comprises a CDR-L1 comprising SEQ ID NO: 38, a CDR-L2 comprising SEQ ID NO: 39 and a CDR-L3 comprising SEQ ID NO: 40;
or
q. The first heavy chain variable region comprises SEQ ID NO: 21 and the first light chain variable region comprises SEQ ID NO: 23; and the second heavy chain variable region comprises SEQ ID NO: 41 and second light chain variable region comprises SEQ ID NO: 43;
or
r. The first heavy chain variable region is encoded by a nucleotide sequence comprising SEQ ID NO: 22 and the first light chain variable region is encoded by a nucleotide sequence comprising SEQ ID NO: 24; and the second heavy chain variable region is encoded by a nucleotide sequence comprising SEQ ID NO: 42 and second light chain variable region is encoded by a nucleotide sequence comprising SEQ ID NO: 44.

In a second aspect of the present invention there is provided a pharmaceutical composition comprising the antibody according to the first aspect of the invention and all its embodiments and one or more pharmaceutically acceptable excipients.

In a third aspect, the invention provides for the antibody according to the first aspect of the invention and all its embodiments or the pharmaceutical composition according to the second aspect of the invention and all its embodiments for use in therapy.

In one embodiment of this third aspect, the use is for the treatment of cancer and/or an infectious disease. In another embodiment, the antibody or the composition according to the invention and all its embodiments are for use in the treatment of cancer concomitantly or sequentially to one or more additional cancer therapies.

In a fourth aspect of the present invention, there is provided for a method for treating a subject afflicted with cancer and/or an infectious disease, comprising administering to the subject a pharmaceutically effective amount of the antibody according to the first aspect of the invention and all its embodiments or the pharmaceutical composition according to the second aspect of the invention and all its embodiments.

In one embodiment of this fourth aspect, the antibody or the composition are administered concomitantly or sequentially to one or more additional cancer therapies.

In a fifth aspect, the invention provides for the use of an antibody according to the first aspect of the invention and all its embodiments or the pharmaceutical composition according to the second aspect of the invention and all its embodiments in the manufacture of a medicament for treating cancer.

In one embodiment of this fifth aspect, the antibody or the composition are administered concomitantly or sequentially to one or more additional cancer therapies.

### BRIEF DESCRIPTION OF THE DRAWINGS

FIG. 1. Schematic representation of acquired immune resistance through selective memory T cell activation versus non-specific T cell activation.

FIG. 2. General representation of a Fab-X and Fab-Y comprising antigen-binding portions and of the resulting bispecific antibody.

FIG. 3. Log 2 fold change in the median fluorescence intensity (MFI) values of CD25 expression on CD8+ T cells in the presence of anti-CD3 (10 ng/mL) stimulation. Samples of the stimulated cultured PBMC were analysed upon treatment with CD137-CD9 bispecific antibodies or control antibodies by flow cytometry and the CD8+ T cell population identified. Log 2 fold changes were calculated for the MFI of CD25 levels in the treated samples relative to the anti-CD3 stimulated controls. N=4 donors, 2 technical replicates±standard error of the mean (SEM).

FIG. 4. Log 2 fold change in the MFI values of CD25 expression on CD8+ T cells in the absence of any stimulation. Samples of the cultured PBMC were analysed upon treatment with CD137-CD9 bispecific antibodies or control antibodies by flow cytometry and the CD8+ T cell population identified. Log 2 fold changes were calculated for the MFI of CD25 levels in the treated samples relative to the controls. N=4 donors, 2 technical replicates±SEM.

FIG. 5. Log 2 fold change in the MFI values of CD71 expression on CD8+ T cells in the presence of anti-CD3 (10 ng/mL) stimulation. Samples of the stimulated cultured PBMC were analysed upon treatment with CD137-CD9 bispecific antibodies or control antibodies by flow cytometry and the CD8+ T cell population identified. Log 2 fold changes were calculated for the MFI of CD71 levels in the treated samples relative to the anti-CD3 stimulated controls. N=4 donors, 2 technical replicates±SEM.

FIG. 6. Log 2 fold change in the WI values of CD71 expression on CD8+ T cells in the absence of any stimulation. Samples of the cultured PBMC were analysed upon treatment with CD137-CD9 bispecific antibodies or control antibodies by flow cytometry and the CD8+ T cell population identified. Log 2 fold changes were calculated for the MFI of CD71 levels in the treated samples relative to the controls. N=4 donors, 2 technical replicates±SEM.

FIG. 7. Log 2 fold change in the MFI values of CD137 expression on CD8+ T cells in the presence of anti-CD3 (10 ng/mL) stimulation. Samples of the stimulated cultured PBMC were analysed upon treatment with CD137-CD9 bispecific antibodies or control antibodies by flow cytometry and the CD8+ T cell population identified. Log 2 fold changes were calculated for the MFI of CD137 levels in the treated samples relative to the anti-CD3 stimulated controls. N=4 donors, 2 technical replicates±SEM.

FIG. 8. Log 2 fold change in the MFI values of CD137 expression on CD8+ T cells in the absence of any stimulation. Samples of the cultured PBMC were analysed upon treatment with CD137-CD9 bispecific antibodies or control antibodies by flow cytometry and the CD8+ T cell population identified. Log 2 fold changes were calculated for the MFI of CD137 levels in the treated samples relative to controls. N=4 donors, 2 technical replicates±SEM.

FIG. 9. Log 2 fold change in the MFI values of CD25 expression on CD4+ T cells in the presence of anti-CD3 (10 ng/mL) stimulation. Samples of the stimulated cultured PBMC were analysed upon treatment with CD137-CD9 bispecific antibodies or control antibodies by flow cytometry and the CD4+ T cell population identified. Log 2 fold changes were calculated for the MFI of CD25 levels in the treated samples relative to the anti-CD3 stimulated controls. N=4 donors, 2 technical replicates±SEM.

FIG. 10. Log 2 fold change in the MFI values of CD25 expression on CD4+ T cells in the absence of any stimulation. Samples of the cultured PBMC were analysed upon treatment with CD137-CD9 bispecific antibodies or control antibodies by flow cytometry and the CD4+ T-cell population identified. Log 2 fold changes were calculated for the MFI of CD25 levels in the treated samples relative to the controls. N=4 donors, 2 technical replicates±SEM.

FIG. 11. Log 2 fold change in the MFI values of CD71 expression on CD4+ T cells in the presence of anti-CD3 (10 ng/mL) stimulation. Samples of the stimulated cultured PBMC were analysed upon treatment with CD137-CD9 bispecific antibodies or control antibodies by flow cytometry and the CD4+ T cell population identified. Log 2 fold changes were calculated for the MFI of CD71 levels in the treated samples relative to the anti-CD3 stimulated controls. N=4 donors, 2 technical replicates±SEM.

FIG. 12. Log 2 fold change in the MFI values of CD71 expression on CD4+ T cells in the absence of any stimulation. Samples of the cultured PBMC were analysed upon treatment with CD137-CD9 bispecific antibodies or control antibodies by flow cytometry and the CD4+ T cell population identified. Log 2 fold changes were calculated for the MFI of CD71 levels in the treated samples relative to the controls. N=4 donors, 2 technical replicates±SEM.

FIG. 13. Log 2 fold change in the MFI values of CD137 expression on CD4+ T cells in the presence of anti-CD3 (10 ng/mL) stimulation. Samples of the stimulated cultured PBMC were analysed upon treatment with CD137-CD9 bispecific antibodies or control antibodies by flow cytometry and the CD4+ T cell population identified. Log 2 fold changes were calculated for the MFI of CD137 levels in the treated samples relative to the anti-CD3 stimulated controls. N=4 donors, 2 technical replicates±SEM.

FIG. 14. Log 2 fold change in the MFI values of CD137 expression on CD4+ T cells in the absence of any stimulation. Samples of the stimulated cultured PBMC were analysed upon treatment with CD137-CD9 bispecific antibodies or control antibodies by flow cytometry and the CD4+ T cell population identified. Log 2 fold changes were calculated for the MFI of CD137 levels in the treated samples relative to the anti-CD3 stimulated controls. N=4 donors, 2 technical replicates±SEM.

FIG. 15. Log 2 fold change in the MFI values of CD25 expression on CD4+ T cells in the presence of SEB (1 μg/mL) stimulation. PBMC cultures were treated with Staphylococcus aureus Enterotoxin B (SEB) at 1 μg/mL for 48 hours in the presence of either the HVEM-CD9 bispecific construct or control constructs followed by flow cytometry for identifying the CD4+ T cell population. Log 2 fold changes were calculated for the MFI of CD25 levels in the treated samples relative to the SEB stimulated controls. N=4 donors, 2 technical replicates±SEM.

FIG. 16. Log 2 fold change in the MFI values of CD25 expression on CD8+ T cells in the presence of SEB (1 μg/mL) stimulation. PBMC cultures were treated with SEB at 1 μg/mL for 48 hours in the presence of either the HVEM-CD9 bispecific antibodies or control antibodies. The samples were then analysed by flow cytometry and the CD8+ T cell population identified. Log 2 fold changes were calculated for the MFI of CD25 levels in the treated samples relative to the SEB stimulated controls. N=4 donors, 2 technical replicates±SEM.

FIG. 17. Log 2 fold change in the MFI values of CD25 expression on CD4+ T cells in the absence of any stimulation. PBMC cultures were cultured for 48 hours in the presence of either the HVEM-CD9 bispecific antibodies or control antibodies. The samples were then analysed by flow cytometry and the CD4+ T cell population identified. Log 2 fold changes were calculated for the MFI of CD25 levels in the treated samples relative to the unstimulated controls. N=4 donors, 2 technical replicates±SEM.

FIG. 18. Log 2 fold change in the MFI values of CD25 expression on CD8+ T cells in the absence of any stimulation. PBMC cultures were cultured for 48 hours in the presence of either the HVEM-CD9 bispecific antibodies or control antibodies. The samples were then analysed by flow cytometry and the CD8+ T-cell population identified. Log 2 fold changes were calculated for the MFI of CD25 levels in the treated samples relative to the anti-CD3 stimulated controls. N=4 donors, 2 technical replicates±SEM.

FIG. 19. Log 2 fold change in the MFI values of CD25 expression on CD4+ memory T cells in the presence of SEB (1 μg/mL) stimulation. PBMC cultures were treated with SEB at 1 μg/mL for 48 hours in the presence of either the HVEM-CD9 bispecific antibodies or control antibodies. The samples were then analysed by flow cytometry and the CD4+ T cell population identified. Log 2 fold changes were calculated for the WI of CD25 levels in the treated samples relative to the SEB stimulated controls. N=4 donors, 2 technical replicates±SEM.

FIG. 20. Log 2 fold change in the MFI values of CD25 expression on CD4+ naïve T cells in the presence of SEB (1 lag/mL) stimulation. PBMC cultures were treated with SEB at 1 μg/mL for 48 hours in the presence of either the HVEM-CD9 bispecific antibodies or control antibodies. The samples were then analysed by flow cytometry and the CD4+ T cell population identified. Log 2 fold changes were calculated for the MFI of CD25 levels in the treated samples relative to the SEB stimulated controls. N=4 donors, 2 technical replicates±SEM.

FIG. 21. Log 2 fold change in the MFI values of CD25 expression on CD8+ memory T cells in the presence of SEB (1 μg/mL) stimulation. PBMC cultures were treated with SEB at 1 μg/mL for 48 hours in the presence of either the HVEM-CD9 bispecific antibodies or control antibodies. The samples were then analysed by flow cytometry and the CD8+ T cell population identified. Log 2 fold changes were calculated for the MFI of CD25 levels in the treated samples relative to the SEB stimulated controls. N=4 donors, 2 technical replicates±SEM.

FIG. 22. Log 2 fold change in the MFI values of CD25 expression on CD8+ naïve T cells in the presence of SEB (1 μg/mL) stimulation. PBMC cultures were treated with SEB at 1 μg/mL for 48 hours in the presence of either the HVEM-CD9 bispecific antibodies or control antibodies. The samples were then analysed by flow cytometry and the CD8+ T cell population identified. Log 2 fold changes were calculated for the MFI of CD25 levels in the treated samples relative to the SEB stimulated controls. N=4 donors, 2 technical replicates±SEM.

FIG. 23. Log 2 fold change in the MFI values of CD71 expression on CD4+ T memory cells in the presence of SEB (1 μg/mL) stimulation. PBMC cultures were treated with SEB at 1 μg/mL for 48 hours in the presence of either the HVEM-CD9 bispecific antibodies or control antibodies. The samples were then analysed by flow cytometry and the CD4+ memory T cell population identified. Log 2 fold changes were calculated for the MFI of CD71 levels in the treated samples relative to the SEB stimulated controls. N=4 donors, 2 technical replicates±SEM.

FIG. 24. Log 2 fold change in the MFI values of CD71 expression on CD8+ T memory cells in the presence of SEB (1 μg/mL) stimulation. PBMC cultures were treated with SEB at 1 μg/mL for 48 hours in the presence of either the HVEM-CD9 bispecific antibodies or control antibodies. The samples were then analysed by flow cytometry and the CD8+ memory T cell population identified. Log 2 fold changes were calculated for the MFI of CD71 levels in the treated samples relative to the SEB stimulated controls. N=4 donors, 2 technical replicates±SEM.

FIG. 25. Log 2 fold change in the MFI values of CD137 expression on CD4+ T memory cells in the presence of SEB (1 μg/mL) stimulation. PBMC cultures were treated with SEB at 1 μg/mL for 48 hours in the presence of either the HVEM-CD9 bispecific antibodies or control antibodies. The samples were then analysed by flow cytometry and the CD4+ memory T cell population identified. Log 2 fold changes were calculated for the MFI of CD137 levels in the treated samples relative to the SEB stimulated controls. N=4 donors, 2 technical replicates±SEM.

FIG. 26. Log 2 fold change in the MFI values of CD137 expression on CD8+ T memory cells in the presence of SEB (1 μg/mL) stimulation. PBMC cultures were treated with SEB at 1 μg/mL for 48 hours in the presence of either the HVEM-CD9 bispecific antibodies or control antibodies. The samples were then analysed by flow cytometry and the CD8+ memory T cell population identified. Log 2 fold changes were calculated for the MFI of CD137 levels in the treated samples relative to the SEB stimulated controls. N=4 donors, 2 technical replicates±SEM.

FIG. 27. Log 2 fold change in the concentration of granzyme B levels using an IntelliCyt® QBead PlexScreen in the supernatant of PBMC cultures in the presence of SEB (1 μg/mL) stimulation. Log 2 fold changes were calculated for the concentrations of granzyme B levels in the samples treated with the antibodies relative to the SEB stimulated controls. N=3 donors, 2 technical replicates±SEM. PBMC cultures were treated with SEB at 1 μg/mL for 48 hours in the presence of either the CD7-CD9 bispecific antibodies or control antibodies. The conditioned media was collected and diluted 40-fold before analysis of the level of granzyme B.

FIG. 28. Log 2 fold change in the concentration of IFNgamma levels using an IntelliCyt® QBead PlexScreen in the supernatant of PBMC cultures in the presence of SEB (1 μg/mL) stimulation. Log 2 fold changes were calculated for the concentrations of IFNgamma levels in the samples treated with the antibodies relative to the SEB stimulated controls. N=3 donors, 2 technical replicates±SEM. PBMC cultures were treated with SEB at 1 μg/mL for 48 hours in the presence of either the CD7-CD9 bispecific antibodies or control antibodies. The conditioned media was collected and diluted 40-old before analysis of the level of IFNgamma.

FIG. 29. Log 2 fold change in the concentration of granzyme B levels using an IntelliCyt® QBead PlexScreen in the supernatant of PBMC cultures in the presence of anti-CD3 (clone UCHT1) stimulation. Log 2 fold changes were calculated for the concentrations of granzyme B levels in the samples treated with the antibodies relative to the SEB stimulated controls. N=3 donors, 2 technical replicates±SEM. PBMC cultures were treated with anti-CD3 at 250 ng/mL for 48 hours in the presence of either the CD7-CD9 bispecific antibodies or control antibodies. The conditioned media was collected and diluted 40-fold before analysis of the level of granzyme B.

FIG. 30. Log 2 fold change in the concentration of IFNgamma levels using an IntelliCyt® QBead PlexScreen in the supernatant of PBMC cultures in the presence of anti-CD3 (clone UCHT1) stimulation. Log 2 fold changes were calculated for the concentrations of IFNgamma levels in the treated samples relative to the SEB stimulated controls. N=3 donors, 2 technical replicates±SEM. PBMC cultures were treated with anti-CD3 at 250 ng/mL for 48 hours in the presence of either the CD7-CD9 bispecific antibodies or control antibodies. The conditioned media was collected and diluted 40-fold before analysis of the level of IFNgamma.

FIG. 31. Log 2 fold change in the MFI values of CD25 expression on CD8+ T cells in the presence of anti-CD3 (10 ng/mL) stimulation. PBMC cultures were treated with soluble anti-CD3 (clone UCHT1) at 10 ng/mL for 48 hours in the presence of either the CD137-CD9 bispecific antibodies, control antibodies or corresponding IgG proteins. The samples were then analysed by flow cytometry and the CD8+ T cell population identified. Log 2 fold changes were calculated for the MFI of CD25 levels in the treated samples relative to the anti-CD3 stimulated controls. N=4 donors, 2 technical replicates±SEM.

FIG. 32. Log 2 fold change in the MFI values of CD25 expression on CD8+ T cells in the absence of any stimulation. PBMC cultures were incubated for 48 hours in the presence of either the CD137-CD9 bispecific antibodies, control antibodies or corresponding IgG proteins. The samples were then analysed by flow cytometry and the CD8+ T cell population identified. Log 2 fold changes were calculated for the MFI of CD25 levels in the treated samples relative to the untreated controls. N=4 donors, 2 technical replicates±SEM

FIG. 33. Log 2 fold change in the MFI values of CD25 expression on CD4+ T cells in the presence of anti-CD3 (10 ng/mL) stimulation. PBMC cultures were treated with soluble anti-CD3 (clone UCHT1) at 10 ng/mL for 48 hours in the presence of either the CD137-CD9 bispecific antibodies, control antibodies or corresponding IgG proteins. The samples were then analysed by flow cytometry and the CD4+ T cell population identified. Log 2 fold changes were calculated for the MFI of CD25 levels in the treated samples relative to the anti-CD3 stimulated controls. N=4 donors, 2 technical replicates±SEM

FIG. 34. Log 2 fold change in the MFI values of CD25 expression on CD4+ T cells in the absence of any stimulation. PBMC cultures were incubated for 48 hours in the presence of either the CD137-CD9 bispecific antibodies, control antibodies or corresponding IgG proteins. The samples were then analysed by flow cytometry and the CD4+ T cell population identified. Log 2 fold changes were calculated for the MFI of CD25 levels in the treated samples relative to the untreated controls. N=4 donors, 2 technical replicates±SEM

FIG. 35. Log 2 fold change in the MFI values of CD71 expression on CD8+ T cells in the presence of anti-CD3 (10 ng/mL) stimulation. PBMC cultures were treated with soluble anti-CD3 (clone UCHT1) at 10 ng/mL for 48 hours in the presence of either the CD137-CD9 bispecific antibodies, control antibodies or corresponding IgG proteins. The samples were then analysed by flow cytometry and the CD8+ T cell population identified. Log 2 fold changes were calculated for the MFI of CD71 levels in the treated samples relative to the anti-CD3 stimulated controls. N=4 donors, 2 technical replicates±SEM

FIG. 36. Log 2 fold change in the MFI values of CD71 expression on CD8+ T cells in the absence of any stimulation. PBMC cultures were incubated for 48 hours in the presence of either the CD137-CD9 bispecific antibodies, control antibodies or corresponding IgG proteins. The samples were then analysed by flow cytometry and the CD8+ T cell population identified. Log 2 fold changes were calculated for the MFI of CD71 levels in the treated samples relative to the untreated controls. N=4 donors, 2 technical replicates±SEM

FIG. 37. Log 2 fold change in the MFI values of CD71 expression on CD4+ T cells in the presence of anti-CD3 (10 ng/mL) stimulation. PBMC cultures were treated with soluble anti-CD3 (clone UCHT1) at 10 ng/mL for 48 hours in the presence of either the CD137-CD9 bispecific antibodies, control antibodies or corresponding IgG proteins. The samples were then analysed by flow cytometry and the CD4+ T cell population identified. Log 2 fold changes were calculated for the MFI of CD71 levels in the treated samples relative to the anti-CD3 stimulated controls. N=4 donors, 2 technical replicates±SEM

FIG. 38. Log 2 fold change in the MFI values of CD71 expression on CD4+ T cells in the absence of any stimulation. PBMC cultures were incubated for 48 hours in the presence of either the CD137-CD9 bispecific antibodies, control antibodies or corresponding IgG proteins. The samples were then analysed by flow cytometry and the CD4+ T cell population identified. Log 2 fold changes were calculated for the MFI of CD71 levels in the treated samples relative to the untreated controls. N=4 donors, 2 technical replicates±SEM.

FIG. 39. Numbers of proliferating CD8+ and CD4+ T cells in the presence of anti-CD3 (10 ng/mL) stimulation. Human PBMC were labeled with Cell Tracen™ Violet (CTV) then incubated in for 4 days with anti-CD3 plus 100 nM of the CD137-CD9 bispecific antibodies or control antibodies. T cell proliferation was assessed by flow cytometry by gating on CD8+ (top plot) and CD4+ (bottom plot) populations and enumeration of CTV low cells. Results are presented as the mean±SEM. Similar data was obtained from a further 3 PBMC donors.

FIG. 40. Numbers of proliferating CD8+ and CD4+ T cells in the presence of anti-CD3 (50 ng/mL) stimulation. Human PBMC were labeled with Cell Trace™ Violet (CTV) then incubated in for 4 days with anti-CD3 plus 100 nM of the HVEM-CD9 bispecific antibodies. T cell proliferation was assessed by flow cytometry by gating on CD8+ and CD4+ populations and enumeration of CTV low cells. Results are presented as the mean±SEM of 5 PBMC donors and statistically significant differences with a p value <0.01 high-lighted (Mann-Whitley test).

FIG. 41. Numbers of proliferating CD8+ and CD4+ T cells in the presence of anti-CD3 (50 ng/mL) stimulation. Human PBMC were labeled with Cell Tracen™ Violet (CTV) then incubated in triplicate wells for 4 days with anti-CD3 plus 100 nM of the CD7-CD9 bispecific antibodies. T cell proliferation was assessed by flow cytometry by gating on CD8+ and CD4+ populations and enumeration of CTV low cells. Results are presented as the mean±SEM of 5 PBMC donors and statistically significant differences with a p value <0.01 high-lighted (Mann-Whitley test).

FIG. 42. Numbers of proliferating CD8+ and CD4+ T cells in the presence of anti-CD3 (10 ng/mL) stimulation. Human PBMC were labeled with Cell Tracen™ Violet (CTV) then incubated in triplicate wells for 4 days with anti-CD3 plus 100, 10 or 1 nM of the CD137-CD9 bispecific antibodies or control antibodies. T cell proliferation was assessed by flow cytometry by gating on CD8+ (top plot) and CD4+ (bottom plot) populations and enumeration of CTV low cells. Results are presented as the mean±SEM and statistically significant differences with a p value <0.01 high-lighted (Mann-Whitley test).

FIG. 43. Log 2 fold change in the number of proliferating CD8+ and CD4+ T cells in the presence of anti-CD3 (50ng/mL) stimulation. Human PBMC were labeled with Cell Tracen™ violet (CTV) then incubated for 6 days with anti-CD3 plus 100 nM of the CD137-CD9 bispecific antibodies or control antibodies. T cell proliferation was assessed by flow cytometry by gating on CD8+ (top plot) and CD4+ (bottom plot) populations and enumeration of CTV-low cells. N=4 donors±SEM.

FIG. 44. Log 2 fold change in the MFI of CD71 on CD8+ and CD4+ T cells in the presence of anti-CD3 (50 ng/mL) stimulation. Human PBMC were incubated in for 6 days with anti-CD3 plus 100 nM of the CD137-CD9 bispecific antibodies or control antibodies. CD25 levels on the T cells was then measured by flow cytometry by gating on CD8+ (top plot) and CD4+ (bottom plot) populations. Log 2 fold changes were calculated for the MFI of CD71 levels in the treated samples relative to the anti-CD3 stimulated controls. N=4 donors±SEM.

FIG. 45. Log 2 fold change in the MFI of CD71 on CD8+ and CD4+ T cells in the presence of anti-CD3 (50ng/mL) plus anti-CD28 (200 ng/mL) stimulation. Human PBMC were incubated for 6 days with anti-CD3 plus 100 nM of the CD137-CD9 bispecific antibodies or control antibodies. CD25 levels on the T cells was then measured by flow cytometry by gating on CD8+ (top plot) and CD4+ (bottom plot) populations. Log 2 fold changes were calculated for the MFI of CD71 levels in the treated samples relative to the anti-CD3 stimulated controls. N=4 donors±SEM.

FIG. 46. Log 2 fold change in the concentration of granzyme B levels in the conditioned medium of PBMC cultures in the presence of SEB (1 μg/mL) stimulation. PBMC cultures were treated with (SEB) at 1 μg/mL for 48 hours in the presence of CD7-CD9 bispecific and control antibodies. The conditioned media were collected and diluted 50-fold before analysis of the level of granzyme B using an IntelliCyt® QBead® PlexScreen. Log 2 fold changes were calculated for the concentration of granzyme B levels in the treated samples relative to the SEB stimulated controls. N=3 donors±SEM.

FIG. 47. NK cell activation and degranulation following co-culture with K562 target cells. Human PBMC cells were co-cultured with K562 target cells at an effector to target ratio (E:T) ratio of 10:1 in the presence of 100 nM of the CD7-CD9 bispecific antibodies and control antibodies for 2 hours at 37° C., 5% CO₂. Each condition was tested in quadruplicate wells. NK cell degranulation was measured by flow cytometry by gating on CD3-CD56+ CD107a+ cells. NK cell activation was measured by gating on CD3-CD56+CD69+ cells. Results from 3 donors were pooled and data is presented as individual donors (black circles) or mean±SEM (horizontal line).

FIG. 48. Melanoma patient PBMC were stimulated with melanoma peptide mix (1 μg/mL) for 6 days, in the presence or absence of IgG molecules (100 nM), CD137-Fab X/CD9-Fab Y (100 nM), or pembrolizumab (1 μg/mL). Unstimulated and peptide-stimulated vehicle controls (1% PBS) were also included. Following 6 days of culture, absolute concentrations of IFNγ and TNFα were quantified in the conditioned medium by Luminex® bead assay and the remaining cells were stained for CD3, CD56 and CD71 marker expression, and CD3-CD56+ natural killer cells, analysed for CD71 activation marker expression by flow cytometry. Data presented as mean±SEM of n=4 or n=5 independent melanoma PBMC donors (cytokine and flow cytometry data respectively). Work performed at Celentyx, Birmingham, UK.

FIG. 49: BioMAP® profile of CD137-CD9 bispecific IgG in the StroHT29 and VascHT29 CRC panels. Individual IgG treatments are shown on the X-axis, and the Y-axis represents a log 2-transformed fold change in the biomarker readouts for the IgG test samples (n=3) divided by vehicle controls (n≥6). The dashed lines indicate the 95% vehicle only controls significance range, and the solid line the effects of pembrolizumab (50 μg/ml) based on the average of cumulative runs in the same assay (n>40). CD137-CD9 bispecific IgG-mediated effects on IFNγ, TNF, IL-2 and collagen III in the StroHT29 model and IFNγ in the VascHT29 model are summarised in each panel, denoted with the prefix Stro or Vasc respectively. Statistically significant changes in biomarker levels are indicated by an asterisk, reflecting a >20% effect compared to the vehicle control (log 2 fold change >0.20-0.33) and a p value <0.01 (unpaired T test).

FIG. 50. Log 2 fold change in the MFI of CD25 expression on CD8+ T-cells in the presence of anti-CD3 (10 ng/mL) stimulation. Samples of the stimulated PBMC were analysed upon treatment with CD137-CD9 bispecific antibodies by flow cytometry and the CD8+ T-cell population identified. Log 2 fold changes were calculated for the MFI of CD25 levels in the treated samples relative to the anti-CD3 stimulated controls. N=3 donors, 3 technical replicates±SEM.

FIG. 51. Log 2 fold change in the MFI of granzyme B levels in the conditioned medium of PBMC cultures in the presence of SEB (1 μg/mL) stimulation. PBMC cultures were treated with SEB at 1 μg/mL for 48 hours in the presence of CD7-CD9 bispecific antibodies. The conditioned medium was collected and diluted 50-fold before analysis of the level of granzyme B using an IntelliCyt® QBead® PlexScreen. Log 2 fold changes were calculated for the MFI of granzyme B levels in the treated samples relative to the SEB stimulated controls. N=3 donors±SEM.

FIG. 52. Log₂ Fold Change in the MFI of CD25 expression on CD4⁺ T cells in the presence of SEB (1 μg/mL) stimulation. PBMC cultures were treated with Staphylococcus aureus Enterotoxin B (SEB) at 1 μg/mL for 48 hours in the presence of the HVEM-CD9 bispecific antibodies. The samples were then analysed by flow cytometry and the CD4⁺ T cell population identified. Log 2 fold changes were calculated for the MFI of CD25 levels in the treated samples relative to the SEB stimulated controls. N=2 donors, 2 technical replicates.±SEM.

### DETAILED DESCRIPTION OF THE INVENTION

The present invention will now be described with respect to particular non-limiting aspects and embodiments thereof and with reference to certain figures and examples.

Technical terms are used by their common sense unless indicated otherwise. If a specific meaning is conveyed to certain terms, definitions of terms will be given in the context of which the terms are used.

Where the term “comprising” is used in the present description and claims, it does not exclude other elements. For the purposes of the present disclosure, the term “consisting of” is considered to be a preferred embodiment of the term “comprising of”.

Where an indefinite or definite article is used when referring to a singular noun, e.g. “a”, “an” or “the”, this includes a plural of that noun unless something else is specifically stated.

As used herein, the terms “treatment”, “treating” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. Treatment thus covers any treatment of a disease in a mammal, particularly in a human, and includes: (a) preventing the disease from occurring in a subject, i.e. a human, which may be predisposed to the disease but has not yet been diagnosed as having it; (b) inhibiting the disease, i.e., arresting its development; and (c) relieving the disease, i.e., causing regression of the disease.

A “therapeutically effective amount” refers to the amount of antibody comprising the distinct antigen-binding portions binding CD9 and another antigen that, when administered to a mammal or other subject for treating a disease, is sufficient to affect such treatment for the disease.

The term “activate” (and grammatical variations thereof) as used herein at least includes the upregulation of specific T cell markers, i.e. increased transcription and/or translation of these markers and/or trafficking of these newly transcribed/translated markers or of any marker already expressed to the cell membrane; the upregulation of specific cytokines, i.e. increased transcription and/or translation of these cytokines and/or release/secretion of these cytokines and includes the induction of proliferation.

The present invention provides for an antibody which comprises at least a first antigen-binding portion binding CD9 and a second antigen-binding portion binding another antigen.

CD9 and in particular human CD9 (Uniprot accession number P21926) was discovered as a human B lymphocyte differentiation antigen and it has been found to be widely expressed on many non-hematopoietic tissues including various cancer. It is also known as Tetraspanin-29, motility-related protein-1, 5H9 antigen, cell growth-inhibiting gene 2 protein, leukocyte antigen MIC3 and MRP-1 (Rappa et al., Oncotarget 6:10, 7970-7991 (2015)). CD9 is a tetraspanin that is broadly expressed in a variety of solid tissues and on a multitude of hematopoietic cells (Nature reviews Cancer (9) 40-55 (2009)). CD9 involvement has been shown in the invasiveness and tumorigenicity of human breast cancer cells (Oncotargets, 6:10 (2015)), the suppression of cell motility and metastasis (J. Exp. Med 177:5 (1993)) and to have a role in T cell activation (J. Exp. Med 184:2 (1994)).

CD9 has also been shown to be present in exosomes (Asia-Pac J Clin Oncol, 2018; 1-9). Exosomes are cell derived nanovesicles with size of 30-120 nm. The molecular composition of exosomes reflects their origin and include unique composition of tetraspanins. Exosomes are thoughts to constitute a potent mode of intercellular communication that is important in the immune response, cell-to-cell spread of infectious agents, and tumour progression.

The sequence of human CD9, including the signal peptide is shown as SEQ ID NO:1 (Table 1).

The second antigen-binding portion preferably binds an antigen expressed on a cell surface. The cell is preferably a cell of the immune system and may be selected from a T cell, a B cell or an NK cell.

The first antigen binding-portion binding CD9 and the second antigen-binding portions are located in the same antibody, i.e. they are part of the same polypeptide chain and/or associate via one or more covalent and/or non-covalent associations (such as the screening format Fab-K_{D}-Fab described herein or the classic heavy and light chain association forming a full IgG antibody) or are covalently linked so as to form one single molecule (such as cross-linking two separately expressed polypeptide chains, optionally via specific cross-linking agents).

In one embodiment, the antibody comprises a first antigen-binding portion binding CD9 and a second antigen-binding portion binding CTLA4. For example, the second antigen-binding portion binding CTLA4 may be the antigen-binding portion of ipilimumab or of tremelimumab.

In another embodiment, the antibody comprises a first antigen-binding portion binding CD9 and a second antigen-binding portion binding PD1. For example, the second antigen-binding portion may be the antigen-binding portion of pembrolizumab or of nivolumab or of cemiplimab.

In another embodiment, the antibody comprises a first antigen-binding portion binding CD9 and a second antigen-binding portion binding PD1L1. For example, the second antigen-binding portion may be the antigen-binding portion of atezolizumab or of avelumab or of durvalumab.

In one other embodiment of the present invention, the antibody comprises a first antigen-binding portion binding CD9 and a second antigen-binding portion binding CD137.

CD137 and in particular human CD137 (Uniprot accession number Q07011) is a T cell costimulatory receptor induced on TCR activation (Nam et al. Curr. Cancer Drug Targets, 5:357-363 (2005)). It is also known by the names 4-1BB ligand receptor, T cell antigen 4-1BB homolog, TNFRSF9 and T cell antigen ILA. In addition to be expressed on activated CD4+ and CD8+ T cells CD137 is also expressed on CD4+ and CD25+ regulatory T-cells, activated natural killer cells and other immune system cells such as monocyte, neutrophils and dendritic cells. The sequence of human CD137, including the signal peptide is shown as SEQ ID NO:2 (Table 1).

In another embodiment of the present invention, the antibody comprises a first antigen-binding portion binding CD9 and a second antigen-binding portion binding HVEM.

The herpes virus entry mediator (HVEM) is a member of the tumour necrosis factor receptor superfamily (TNFRSF), and it is also known as TNFRSF14 or CD270 Uniprot reference Q92956. HVEM is a bidirectional switch regulating T cell activation in a costimulatory or coinhibitory fashion whose outcome depends on the binding partner. HVEM can act as both receptor and ligand, the binding of endogenous ligand LIGHT or agonist antibodies to HVEM delivers a costimulatory signal; whereas the binding of HVEM to BTLA (IgSF) or CD160 on Effector T cells delivers a coinhibitory signal. The sequence of human HVEM, including the signal peptide is shown as SEQ ID NO:3 (Table 1).

In another embodiment of the present invention, the antibody comprises a first antigen-binding portion binding CD9 and a second antigen-binding portion binding CD7.

CD7 and in particular human CD7 (Uniprot accession number P09564) is a transmembrane protein which is a member of the immunoglobulin superfamily. This protein is found on thymocytes and mature T cells. It plays an essential role in T cell signal transduction, T cell interactions and also in T cell/B cell interaction during early lymphoid development (Immunologic Research 24, 31-52 (2001)). The sequence of human CD7, including the signal peptide is shown as SEQ ID NO:4 (Table 1).

TABLE 1

| | |
|---|---|
| SEQ ID NO: 1 | MPVKGGTKCIKYLLFGFNFIFWLAGIAVLAIGLWLRFDSQTKSIFEQETN |
| Human CD9 | NNNSSFYTGVYILIGAGALMMLVGFLGCCGAVQESQCMLGLFFGFLLVI |
| | FAIEIAAAIWGYSHKDEVIKEVQEFYKDTYNKLKTKDEPQRETLKAIHYAL |
| | NCCGLAGGVEQFISDICPKKDVLETFTVKSCPDAIKEVFDNKFHIIGAVGI |
| | GIAVVMIFGMIFSMILCCAIRRNREMV |
| SEQ ID NO: 2 | MGNSCYNIVATLLLVLNFERTRSLQDPCSNCPAGTFCDNNRNQICSPC |
| Human CD137 | PPNSFSSAGGQRTCDICRQCKGVFRTRKECSSTSNAECDCTPGFHCL |
| | GAGCSMCEQDCKQGQELTKKGCKDCCFGTFNDQKRGICRPWTNCSL |
| | DGKSVLVNGTKERDVVCGPSPADLSPGASSVTPPAPAREPGHSPQIIS |
| | FFLALTSTALLFLLFFLTLRFSVVKRGRKKLLYIFKQPFMRPVQTTQEED |
| | GCSCRFPEEEEGGCEL |
| SEQ ID NO: 3 | MEPPGDWGPPPWRSTPKTDVLRLVLYLTFLGAPCYAPALPSCKEDEY |
| Human HVEM | PVGSECCPKCSPGYRVKEACGELTGTVCEPCPPGTYIAHLNGLSKCLQ |
| | CQMCDPAMGLRASRNCSRTENAVCGCSPGHFCIVQDGDHCAACRAY |
| | ATSSPGQRVQKGGTESQDTLCQNCPPGTFSPNGTLEECQHQTKCSW |
| | LVTKAGAGTSSSHWVWWFLSGSLVIVIVCSTVGLIICVKRRKPRGDVVK |
| | VIVSVQRKRQEAEGEATVIEALQAPPDVTTVAVEETIPSFTGRSPNH |
| SEQ ID NO: 4 | MAGPPRLLLLPLLLALARGLPGALAAQEVQQSPHCTTVPVGASVNITCS |
| Human CD7 | TSGGLRGIYLRQLGPQPQDIIYYEDGVVPTTDRRFRGRIDFSGSQDNLTI |
| | TMHRLQLSDTGTYTCQAITEVNVYGSGTLVLVTEEQSQGWHRCSDAP |
| | PRASALPAPPTGSALPDPQTASALPDPPAASALPAALAVISFLLGLGLGV |
| | ACVLARTQIKKLCSWRDKNSAACWYEDMSHSRCNTLSSPNQYQ |
| SEQ ID NO: 5 | GFSLSTYAMS |
| CDR-H1 | |
| VR8475 | |
| SEQ ID NO: 6 | AIWSGGTTDYTSWAKG |
| CDR-H2 | |
| VR8475 | |
| SEQ ID NO: 7 | MAVIFNAYTFDS |
| CDR-H3 | |
| VR8475 | |
| SEQ ID NO: 8 | QASQNIYSYLA |
| CDR-L1 | |
| VR8475 | |
| SEQ ID NO: 9 | GASTLAS |
| CDR-L2 | |
| VR8475 | |
| SEQ ID NO: 10 | QQGHSSGNVGNNV |
| CDR-L3 | |
| VR8475 | |
| SEQ ID NO: 11 | GFSLSSYAMG |
| CDR-H1 | |
| VR7272 | |
| SEQ ID NO: 12 | AIGSITATGYARWAKG |
| CDR-H2 | |
| VR7272 | |
| SEQ ID NO: 13 | EIYVGSAYAFDI |
| CDR-H3 | |
| VR7272 | |
| SEQ ID NO: 14 | QASQSISNYLA |
| CDR-L1 | |
| VR7272 | |
| SEQ ID NO: 15 | LASTLAS |
| CDR-L2 | |
| VR7272 | |
| SEQ ID NO: 16 | QQGYIDNVNKG |
| CDR-L3 | |
| VR7272 | |
| SEQ ID NO: 17 | QSVEESGGRLVTPGTPLTLTCTVSGFSLSTYAMSWVRQAPGKGLEWI |
| VH VR8475 | GAIWSGGTTDYTSWAKGRFTISKASTTVDLKITSPTTEDTATYFCVRMA |
| | VIFNAYTFDSWGPGTLVTVSS |
| SEQ ID NO: 18 | AAGCTTCGAAGCCACCATGGAGACTGGGCTGCGCTGGCTTCTCCTG |
| VH nucl. | GTCGCTGTGCTCAAAGGTGTCCAGTGTCAGTCGGTGGAGGAGTCC |
| VR8475 | GGGGGTCGCCTGGTCACGCCTGGGACACCCCTGACACTCACCTGC |
| | ACAGTCTCTGGATTCTCCCTCAGTACGTATGCAATGAGCTGGGTCC |
| | GCCAGGCTCCAGGGAAGGGGCTGGAGTGGATCGGAGCGATTTGGA |
| | GTGGTGGTACCACGGACTACACGAGCTGGGCGAAAGGCCGATTCA |
| | CCATCTCCAAAGCCTCGACCACGGTGGATCTGAAAATCACCAGTCC |
| | GACAACCGAGGACACGGCCACCTATTTCTGTGTCAGAATGGCAGTG |
| | ATTTTTAATGCTTACACTTTTGATTCCTGGGGCCCAGGCACCCTGGT |
| | CACCGTCTCGAGT |
| SEQ ID NO: 19 | AYDMTQTPSSVSAAVGGTVTIKCQASQNIYSYLAWYQQKPGQRPKLLI |
| VL VR8475 | YGASTLASGVPSRFKGSGSGTDFTLTISDLECDDAATYYCQQGHSSGN |
| | VGNNVFGGGTEVVVK |
| SEQ ID NO: 20 | AAGCTTCGAAGCCACCATGAACATGAGGGCCCCCACTCAGCTGCTG |
| VL nucl. | GGGCTCCTGCTGCTCTGGCTCCCAGGTGCCAGATGTGCCTATGATA |
| VR8475 | TGACCCAGACTCCATCCTCCGTGTCTGCAGCTGTGGGAGGCACAGT |
| | CACCATCAAGTGCCAGGCCAGTCAGAACATTTACAGTTACTTAGCCT |
| | GGTATCAGCAGAAACCAGGGCAGCGTCCCAAGCTCCTGATCTATGG |
| | TGCGTCCACTCTGGCATCTGGGGTCCCATCGCGGTTCAAAGGCAGT |
| | GGATCTGGGACAGATTTCACTCTCACCATCAGCGACCTGGAGTGTG |
| | ACGATGCTGCCACTTACTACTGTCAACAGGGCCATAGTAGTGGTAAT |
| | GTTGGAAATAATGTTTTCGGCGGAGGGACCGAGGTGGTGGTCAAAC |
| | GTACG |
| SEQ ID NO: 21 | QSLEESGGRLVTPGTPLTLTCTVSGFSLSSYAMGWVRQAPGKGLEWI |
| VH VR7272 | GAIGSITATGYARWAKGRFSISKTSTTVDLKMTSPTTEDTATYFCAREIY |
| | VGSAYAFDIWGPGTLVTVSS |
| SEQ ID NO: 22 | AAGCTTCGAAGCCACCATGGAGACTGGGCTGCGCTGGCTTCTCCTG |
| VH nucl. | GTCGCTGTGCTCAAAGGTGTCCAGTGTCAGTCGCTGGAGGAGTCC |
| VR7272 | GGGGGTCGCCTGGTCACGCCTGGGACACCCCTGACACTCACCTGC |
| | ACAGTCTCTGGATTCTCCCTCAGTAGCTATGCAATGGGCTGGGTCC |
| | GCCAGGCTCCAGGGAAGGGGCTGGAGTGGATCGGAGCCATTGGTA |
| | GTATTACTGCCACTGGCTACGCGCGCTGGGCAAAAGGCCGATTCAG |
| | CATCTCCAAGACCTCGACCACGGTGGATCTGAAAATGACCAGTCCG |
| | ACAACCGAGGACACGGCCACCTATTTCTGTGCCAGAGAGATTTATG |
| | TTGGGTCTGCTTATGCCTTTGACATCTGGGGCCCAGGCACCCTGGT |
| | CACCGTCTCGAGT |
| SEQ ID NO: 23 | AYDMTQTPASVEVAVGDTVTIKCQASQSISNYLAWYQQKPGQPPKLLIY |
| VL VR7272 | LASTLASGVPSRFKGSGSGTEFTLTISDLECADAATYYCQQGYIDNVNK |
| | GFGGGTEVVVK |
| SEQ ID NO: 24 | AAGCTTCGAAGCCACCATGGACACGAGGGCCCCCACTCAGCTGCT |
| VL nucl. | GGGGCTCCTGCTGCTCTGGCTCCCAGGTGCCAGATGTGCCTATGAT |
| VR7272 | ATGACCCAGACTCCAGCCTCTGTGGAGGTAGCTGTGGGAGACACTG |
| | TCACCATCAAGTGTCAGGCCAGTCAGAGCATTAGTAACTACTTAGCC |
| | TGGTATCAGCAGAAACCAGGGCAGCCTCCCAAGCTCCTGATCTATC |
| | TGGCATCTACTCTGGCATCTGGGGTCCCATCGCGGTTCAAAGGCAG |
| | TGGATCTGGGACAGAGTTCACTCTCACCATCAGCGACCTGGAGTGT |
| | GCCGATGCTGCCACTTACTATTGTCAACAGGGTTATATTGATAATGT |
| | TAATAAAGGTTTCGGCGGAGGGACCGAGGTGGTGGTCAAACGTAC |
| | G |
| SEQ ID NO: 25 | GFSLSTYDMI |
| CDR-H1 | |
| VR7660 | |
| SEQ ID NO: 26 | VITKTTTTYYANWAKG |
| CDR-H2 | |
| VR7660 | |
| SEQ ID NO: 27 | DADDAVYYYFDI |
| CDR-H3 | |
| VR7660 | |
| SEQ ID NO: 28 | QASQSIGSGLA |
| CDR-L1 | |
| VR7660 | |
| SEQ ID NO: 29 | AASTLTS |
| CDR-L2 | |
| VR7660 | |
| SEQ ID NO: 30 | QEGVSRGNIDNA |
| CDR-L3 | |
| VR7660 | |
| SEQ ID NO: 31 | QSVEESGGRLVTPGTPLTLTCTVAGFSLSTYDMIWVRQAPGKGLEYIG |
| VH VR7660 | VITKTTTTYYANWAKGRFTISKTSTTVDLKMTSPTTEDTATYFCARDAD |
| | DAVYYYFDIWGPGTLVTVSS |
| SEQ ID NO: 32 | AAGCTTCGAAGCCACCATGGAGACTGGGCTGCGCTGGCTTCTCCTG |
| VH nucl. | GTCGCTGTGCTCAAAGGTGTCCAGTGTCAGTCGGTGGAGGAGTCC |
| VR7660 | GGGGGTCGCCTGGTCACGCCTGGGACACCCCTGACACTCACCTGC |
| | ACGGTCGCTGGATTCTCCCTCAGTACCTACGACATGATCTGGGTCC |
| | GCCAGGCTCCAGGGAAGGGGCTGGAATACATCGGAGTCATTACTAA |
| | AACTACTACCACATACTACGCGAACTGGGCGAAAGGCCGATTCACC |
| | ATCTCCAAAACCTCGACCACGGTGGATCTGAAAATGACCAGTCCGA |
| | CAACCGAGGACACGGCCACCTATTTCTGTGCCAGAGATGCTGATGA |
| | TGCTGTTTATTACTACTTTGACATCTGGGGCCCAGGCACCCTGGTCA |
| | CCGTCTCGAGT |
| SEQ ID NO: 33 | AYDMTQTPASAEVAVGGTVTINCQASQSIGSGLAWYQQKPGQRPKLLI |
| VL VR7660 | YAASTLTSGVSSRFKGSGSGTEFTLTISGVECADAATYYCQEGVSRGNI |
| | DNAFGGGTEVVVK |
| SEQ ID NO: 34 | AAGCTTCGAAGCCACCATGGACATGAGGGCCCCCACTCAGCTGCTG |
| VL nucl. | GGGCTCCTGCTGCTCTGGCTCCCAGGTGCCAGATGTGCCTATGATA |
| VR7660 | TGACCCAGACTCCAGCCTCTGCGGAGGTAGCTGTGGGAGGCACAG |
| | TCACCATCAATTGCCAGGCCAGTCAGAGCATTGGCAGTGGTTTGGC |
| | CTGGTATCAGCAGAAACCAGGGCAGCGTCCCAAACTCCTGATCTAT |
| | GCTGCATCCACTCTGACATCTGGGGTCTCATCGCGGTTCAAAGGCA |
| | GTGGATCTGGGACAGAGTTCACTCTCACCATCAGCGGCGTGGAGTG |
| | TGCCGATGCTGCCACTTACTACTGTCAAGAGGGTGTTAGTCGTGGT |
| | AATATTGATAATGCCTTCGGCGGAGGGACCGAGGTGGTGGTCAAAC |
| | GTACG |
| SEQ ID NO: 35 | GFSLSSFAMC |
| CDR-H1 | |
| VR8850 | |
| SEQ ID NO: 36 | IINTGGSAYYASWATG |
| CDR-H2 | |
| VR8850 | |
| SEQ ID NO: 37 | GNGYAGYGYDGFDP |
| CDR-H3 | |
| VR8850 | |
| SEQ ID NO: 38 | QASQSITSWLS |
| CDR-L1 | |
| VR8850 | |
| SEQ ID NO: 39 | AASKLTF |
| CDR-L2 | |
| VR8850 | |
| SEQ ID NO: 40 | QSNYGSSSAYGA |
| CDR-L3 | |
| VR8850 | |
| SEQ ID NO: 41 | QSVEESGGRLVTPGTPLTLTCTVSGFSLSSFAMCWVRQAPGKGLEYIG |
| VH VR8850 | IINTGGSAYYASWATGRFTISKTSTTVDLKISSPTTEDTATYFCARGNGY |
| | AGYGYDGFDPWGPGTLVTVSS |
| SEQ ID NO: 42 | AAGCTTCGAAGCCACCATGGAGACTGGGCTGCGCTGGCTTCTCCTG |
| VH nucl. | GTCGCTGTGCTCAAAGGTGTCCAGTGTCAGTCGGTGGAGGAGTCC |
| VR8850 | GGGGGTCGCCTGGTCACGCCGGGGACACCCCTGACACTCACCTGC |
| | ACAGTCTCTGGATTCTCCCTCAGTAGCTTTGCAATGTGCTGGGTCC |
| | GCCAGGCTCCAGGGAAGGGACTGGAATACATCGGAATCATTAATAC |
| | TGGTGGTAGCGCATACTACGCGAGCTGGGCGACAGGCCGATTCAC |
| | CATCTCCAAAACCTCGACCACGGTGGATCTGAAAATCTCCAGTCCG |
| | ACAACCGAGGACACGGCCACCTATTTCTGTGCCAGAGGAAATGGTT |
| | ATGCTGGTTATGGTTATGATGGTTTTGATCCCTGGGGCCCAGGCAC |
| | CCTGGTCACCGTCTCGAGT |
| SEQ ID NO: 43 | DIVMTQTPASVSEPVGGTVTIKCQASQSITSWLSWYQQKPGQPPKLLIY |
| VL VR8850 | AASKLTFGVSSRFRGSGSGTEYTLTISDLECADAATYYCQSNYGSSSA |
| | YGAFGGGTEVVVK |
| SEQ ID NO: 44 | AAGCTTCGAAGCCACCATGGACACGAGGGCCCCCACTCAGCTGCT |
| VL nucl. | GGGGCTCCTGCTGCTCTGGCTCCCAGGTGCCAGATGTGCTGACATT |
| VR8850 | GTGATGACCCAGACTCCAGCCTCCGTGTCTGAACCTGTGGGAGGCA |
| | CAGTCACCATCAAGTGCCAGGCCAGTCAGAGCATTACCAGTTGGTT |
| | ATCCTGGTATCAGCAGAAACCAGGGCAGCCTCCCAAGCTCCTGATC |
| | TACGCGGCATCCAAACTGACATTTGGGGTCTCATCAAGATTCAGAG |
| | GCAGTGGATCTGGGACAGAGTACACTCTCACCATCAGCGACCTGGA |
| | GTGTGCCGATGCTGCCACTTACTACTGTCAAAGCAATTATGGTAGTA |
| | GTAGTGCTTATGGGGCTTTCGGCGGAGGGACCGAGGTGGTGGTCA |
| | AACGTACG |

Within the present invention, unless recited otherwise, human CD9, human CD137, human HVEM and human CD7 and are always intended to be included in the term “CD9”, “CD137”, “HVEM” and “CD7”. However, unless “human CD9”, “human CD137”, “human HVEM” or “human CD7” are explicitly used, the terms “CD9”, “CD137”, “HVEM” or “CD7” include the same targets in other species, especially non-primate (e.g. rodents) and non-human primate (such as cynomolgus monkey) species.

The present invention therefore provides for an antibody comprising a first antigen-binding portion binding human CD9 and a second antigen-binding portion binding human CD137 or human HVEM or human CD7. The first and the second antigen-binding portions are located on the same antibody i.e. they are part of the same polypeptide chain and/or associate via one or more covalent and/or non-covalent associations (such as the screening format Fab-K_{D}-Fab described herein or the classic heavy and light chain association forming a full IgG antibody) or are covalently linked so as to form one single molecule (such as cross-linking two separately expressed polypeptide chains, optionally via specific cross-linking agents).

The present invention also provides for an antibody comprising a first antigen-binding portion binding human CD9 as defined in SEQ ID NO: 1 or from amino acid 2 to 228 of SEQ ID NO: 1, alternatively from amino acid 34 to 55 of SEQ ID NO: 1 or alternatively and preferably the first antigen-binding portion binds within amino acid 112 to 195 of SEQ ID NO: 1 and a second antigen-binding portion binding human CD137 as defined in SEQ ID NO: 2 or from amino acid 24 to 186 of SEQ ID NO: 2. The first and the second antigen-binding portions are located on the same antibody, i.e. they are part of the same polypeptide chain, they associate via one or more non-covalent and/or covalent associations or are linked so as to form one single molecule.

Antibodies against CD137 have been disclosed in several US granted patents such as U.S. Pat. No. 7,288,638 or U.S. Pat. No. 6,887,673. Urelumab is an anti-CD137 fully human IgG4 monoclonal antibody currently in the clinics. In one embodiment, the present invention discloses an antibody comprising a first antigen-binding portion binding human CD137, which is the antigen-binding portion of urelumab, and a second antigen-binding portion binding human CD9.

The monoclonal antibody of the present invention, upon binding of CD137 and CD9, stimulates T cell activation, i.e. further activates T cells and enhances induction of T cell proliferation, and in particular, the monoclonal antibody comprising a first antigen-binding portion binding CD137 and a second antigen-binding portion biding CD9 further activates T cells and enhance induction of T cell proliferation in the presence of an anti-CD3 stimulation. More specifically, the monoclonal antibody comprising a first antigen-binding portion binding CD137 and a second antigen-binding portion binding CD9 further activates T cells and enhance induction of T cell proliferation in the presence of an anti-CD3 stimulation but it does not activate unstimulated T cells. More specifically, the T cell is at least a CD4+ T cell or at least a CD8+ T cell or a mixture thereof.

Hence, the present invention provides for a monoclonal antibody comprising a first antigen-binding portion binding CD137 and a second antigen-binding portion binding CD9 capable of activating T cells in the presence of an anti-CD3 stimulation wherein the further activation of T cell is measured as an upregulation of T cell markers and the enhancement of T cell proliferation.

Specific markers of T cell activation and proliferation include but are not limited to the upregulation of CD25, CD71 and CD137.

In one preferred embodiment of the present invention, the monoclonal antibody comprising a first antigen-binding portion binding CD137 and a second antigen-binding portion binding CD9 is capable of activating T cells in the presence of an anti-CD3 stimulation wherein activating T cell results in an upregulation of CD71, CD25 and CD137.

The present invention also provides for an antibody comprising a first antigen-binding portion binding human CD9 as defined in SEQ ID NO: 1 or from amino acid 2 to 228 of SEQ ID NO: 1, alternatively from amino acid 34 to 55 of SEQ ID NO: 1 or alternatively and preferably the first antigen-binding portion binds within amino acid 112 to 195 of SEQ ID NO: 1 and a second antigen-binding portion binding human HVEM as defined in SEQ ID NO: 3 or from amino acid 39 to 283 of SEQ ID NO: 3 or from amino acid 39 to 202 of SEQ ID NO: 3. The first and the second antigen-binding portions are located on the same antibody, i.e. they are part of the same polypeptide chain, they associate via one or more non-covalent and/or covalent associations or are linked so as to form one single molecule.

The monoclonal antibody of the present invention, upon binding of CD9 and HVEM, stimulates T cell activation, i.e. further activates T cells and enhance induction of T cell proliferation, and in particular, the monoclonal antibody comprising a first antigen-binding portion binding HVEM and a second antigen-binding portion biding CD9 further activates T cells and enhance induction of T cell proliferation in the presence of SEB stimulation. More specifically, the monoclonal antibody comprising a first antigen-binding portion binding CD9 and a second antigen-binding portion binding HVEM further activates T cells and enhance induction of T cell proliferation in the presence of SEB stimulation but it does not activate unstimulated T cells. More specifically, the T cell is at least a CD4+ T cell or at least a CD8+ T cell or a mixture thereof, more preferably a memory CD4+ T cell or a memory CD8+ T cell.

Hence, the present invention provides for a monoclonal antibody comprising a first antigen-binding portion binding CD9 and a second antigen-binding portion binding HVEM capable of activating T cells in the presence of SEB stimulation wherein the further activation of T cell is measured as an upregulation of T cell markers and the enhancement of T cell proliferation, more preferably memory T cell proliferation.

Specific markers of T cell activation and proliferation include but are not limited to the upregulation of CD25, CD137 and CD71.

In one preferred embodiment of the present invention, the monoclonal antibody comprising a first antigen-binding portion binding CD9 and a second antigen-binding portion binding HVEM is capable of activating T cells in the presence of SEB stimulation wherein activating T cell results in an upregulation of CD137, CD71 and CD25.

The present invention also provides for an antibody comprising a first antigen-binding portion binding human CD9 as defined in SEQ ID NO: 1 or from amino acid 2 to 228 of SEQ ID NO: 1, alternatively from amino acid 34 to 55 of SEQ ID NO: 1 or alternatively and preferably the first antigen-binding portion binds within amino acid 112 to 195 of SEQ ID NO: 1 and a second antigen-binding portion binding human CD7 as defined in SEQ ID NO: 4 or from amino acid 26 to 240 of SEQ ID NO: 4 or from or from amino acid 26 to 180 of SEQ ID NO:4. The first and the second antigen-binding portions are located on the same antibody, i.e. they are part of the same polypeptide chain, they associate via one or more non-covalent and/or covalent associations or are linked so as to form one single molecule.

The monoclonal antibody of the present invention, upon binding of CD9 and CD7, stimulates T cell activation, i.e. further activates T cells and enhance induction of T cell proliferation, and in particular, the monoclonal antibody comprising a first antigen-binding portion binding CD9 and a second antigen-binding portion biding CD7 further activates T cells and enhance induction of T cell proliferation in the presence of an anti-CD3 stimulation or in the presence of SEB. More specifically, the monoclonal antibody comprising a first antigen-binding portion binding CD9 and a second antigen-binding portion binding CD7 further activates T cells and enhance induction of T cell proliferation in the presence of an anti-CD3 stimulation or SEB stimulation but it does not activate unstimulated T cells. More specifically, the T cell is at least a CD4+ T cell or at least a CD8+ T cell or a mixture thereof.

Hence, the present invention provides for a monoclonal antibody comprising a first antigen-binding portion binding CD9 and a second antigen-binding portion binding CD7 capable of activating T cells in the presence of an anti-CD3 stimulation wherein the further activation of T cell is measured as an upregulation of T cell markers and the enhancement of T cell proliferation.

Upregulation or enhancement of cytokine production includes but is not limited to the upregulation of granzyme B and/or IFNgamma.

In one preferred embodiment of the present invention, the monoclonal antibody comprising a first antigen-binding portion binding CD9 and a second antigen-binding portion binding CD7 is capable of upregulating or enhancing cytokine production and/or enhancing T cell proliferation in the presence of a super antigen such as SEB or an anti-CD3 stimulation wherein upregulating or enhancing cytokine production results in an upregulation of granzyme B and/or IFNgamma.

The term “antibody” as used herein include whole immunoglobulin molecules and antigen-binding portions of immunoglobulin molecules associated via non-covalent and/or covalent associations or linked together, optionally via a linker.

In another embodiment, the antigen-biding portions comprised in the antibody are functionally active fragments or derivatives of a whole immunoglobulin and may be, but are not limited to, VH, VL, VHH, Fv, scFv fragment (including dsscFv), Fab fragments, modified Fab fragments, Fab′ fragments, F(ab′)₂ fragments, Fv and epitope-binding fragments of any of the above.

Other antibody fragments include those described in WO2005003169, WO2005003170, WO2005003171, WO2009040562 and WO2010035012. Functionally active fragments or derivative of a whole immunoglobulin and methods of producing them are well known in the art, see for example Verma et al., 1998, Journal of Immunological Methods, 216, 165-181; Adair and Lawson, 2005. Therapeutic antibodies. Drug Design Reviews—Online 2(3):209-217.

In one embodiment of the invention each of the antigen-binding portion is independently selected from a Fab, a Fab′, a scFv or a VHH.

In one embodiment, the first antigen-binding portion binding CD9 is a Fab whilst the second antigen-binding portion binding another antigen, such as an antigen expressed on T cells, is a scFv. In another embodiment, the first antigen-binding portion binding CD9 is a scFv and the second antigen-binding portion binding another antigen, such as an antigen expressed on T cells, is a Fab. In another embodiment, both antigen-binding portions are a Fab or scFv. In further variants of this embodiment, the second antigen binding portion may bind an antigen secreted by T cell or another immune system cell such as B cells or NK cells or an antigen expressed on a cell such as an immune system cell.

In one embodiment, the antibody comprises a first antigen-binding portion binding CD9 and a second antigen-binding portion binding CTLA4 or PD1 or PDL1, wherein each of the antigen-binding portions are independently selected from a scFv or a Fab. For example, when the second antigen-binding portion binds is a Fab, it may be the Fab portion of ipilimumab, tremelimumab, pembrolizumab, nivolumab, cemiplimab, atezolizumab, avelumab or of durvalumab. In one embodiment, the antigen-binding portion binding CD9 is a Fab whilst the antigen-binding portion binding CD137 is a scFv. In another embodiment, the antigen-binding portion binding CD137 is a Fab whilst the antigen-binding portion binding CD9 is a scFv. In another embodiment, both antigen-binding portions are a Fab or scFv.

In one preferred embodiment, the antibody is monoclonal, which means that the antigen-binding portions comprised therein are all monoclonal. Therefore, in one preferred embodiment of the present invention, there is provided a monoclonal antibody comprising a first antigen-binding portion binding CD9 and a second antigen-binding portion binding CD137. Preferably, this antibody is capable of further activating T cells and/or enhancing induction of T cell proliferation in the presence of an anti-CD3 stimulation wherein activating T cell results in an upregulation of CD71, CD25 and CD137.

In one embodiment, the antigen-binding portion binding CD9 is a Fab whilst the antigen-binding portion binding HVEM is a scFv. In another embodiment, the antigen-binding portion binding HVEM is a Fab whilst the antigen-binding portion binding CD9 is a scFv. In another embodiment, both antigen-binding portions are a Fab or scFv.

In one preferred embodiment, the antibody is monoclonal, which means that the antigen-binding portions comprised therein are all monoclonal. Therefore, in one preferred embodiment of the present invention, there is provided a monoclonal antibody comprising a first antigen-binding portion binding CD9 and a second antigen-binding portion binding HVEM. Preferably, this antibody is capable of further activating T cells and/or enhancing induction of T cell proliferation in the presence of SEB stimulation wherein activating T cell results in an upregulation of CD71, CD137 and CD25.

In one embodiment, the antigen-binding portion binding CD9 is a Fab whilst the antigen-binding portion binding CD7 is a scFv. In another embodiment, the antigen-binding portion binding CD7 is a Fab whilst the antigen-binding portion binding CD9 is a scFv. In another embodiment, both antigen-binding portions are a Fab or scFv.

In one preferred embodiment, the antibody is monoclonal, which means that the antigen-binding portions comprised therein are all monoclonal. Therefore, in one preferred embodiment of the present invention, there is provided a monoclonal antibody comprising a first antigen-binding portion binding CD9 and a second antigen-binding portion binding CD7. Preferably, this antibody is capable of upregulating or enhancing cytokine production and/or enhancing induction of T cell proliferation in the presence of a super antigen such as SEB or an anti-CD3 stimulation wherein upregulating or enhancing cytokine production results in an upregulation of granzyme B and/or IFNgamma.

Monoclonal antibodies may be prepared by any method known in the art such as the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp 77-96, Alan R Liss, Inc., 1985).

Antibodies for use in the invention may also be generated using single lymphocyte antibody methods by cloning and expressing immunoglobulin variable region cDNAs generated from single lymphocytes selected for the production of specific antibodies by for example the methods described by Babcook, J. et al, 1996, Proc. Natl. Acad. Sci. USA 93(15):7843-78481; WO92/02551; WO2004/051268 and WO2004/106377.

The antibodies of the present invention can also be generated using various phage display methods known in the art and include those disclosed by Brinkman et al. (in J. Immunol. Methods, 1995, 182: 41-50), Ames et al. (J. Immunol. Methods, 1995, 184: 177-186), Kettleborough et al. (Eur. J. Immunol. 1994, 24:952-958), Persic et al. (Gene, 1997 187 9-18), Burton et al. (Advances in Immunology, 1994, 57: 191-280) and WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. When the antigen-binding portions comprised in the antibody are functionally active fragments or derivatives of a whole immunoglobulin such as single chain antibodies, they may be made such as those described in U.S. Pat. No. 4,946,778 which can also be adapted to produce single chain antibodies binding to CD9 and another antigen, such as CD137, HVEM and CD7. Transgenic mice, or other organisms, including other mammals, may be used to express antibodies, including those within the scope of the invention.

The antibody of the present invention may be chimeric, human or humanised.

Chimeric antibodies are those antibodies encoded by immunoglobulin genes that have been genetically engineered so that the light and heavy chain genes are composed of immunoglobulin gene segments belonging to different species.

Humanized, antibodies are antibody molecules from non-human species having one or more complementarity determining regions (CDRs) from the non-human species and a framework region from a human immunoglobulin molecule (see, e.g. US5,585,089; WO91/09967). Preferably the antibody of the present invention is humanized.

In one embodiment of the present invention, there is provided an antibody, preferably a monoclonal antibody, comprising a first antigen-binding portion binding CD9 and a second antigen-binding portion binding another antigen, such as antigen expressed on T cells, wherein the antibody is humanised. In further variants of this embodiment, the second antigen binding portion may bind an antigen secreted by T cell or another immune system cell such as B cells or NK cells or an antigen expressed on a cell such as an immune system cell.

In one embodiment of the present invention, there is provided an antibody, preferably a monoclonal antibody, comprising a first antigen-binding portion binding CD9 and a second antigen-binding portion binding another antigen, such as CD137, HVEM or CD7, wherein the antibody is humanised. More preferably this antibody is capable of activating T cells and/or enhancing induction of T cell proliferation in the presence of an anti-CD3 stimulation wherein activating T cell results in an upregulation of CD71, CD25 and CD137 and/or in an upregulation of granzyme B and/or IFNgamma.

In humanized antibodies, the heavy and/or light chain contains one or more CDRs (including, if desired, one or more modified CDRs) from a donor antibody (e.g. a murine monoclonal antibody) grafted into a heavy and/or light chain variable region framework of an acceptor antibody (e.g. a human antibody). For a review, see Vaughan et al, Nature Biotechnology, 16, 535-539, 1998. In one embodiment, rather than the entire CDR being transferred, only one or more of the specificity determining residues from any one of the CDRs described herein above are transferred to the human antibody framework (see, for example, Kashmiri et al., 2005, Methods, 36, 25-34). When the CDRs or specificity determining residues are grafted, any appropriate acceptor variable region framework sequence may be used having regard to the class/type of the donor antibody from which the CDRs are derived, including mouse, primate and human framework regions. Preferably, the humanized antibody according to the invention comprises a variable domain comprising human acceptor framework regions as well as one or more of the CDRs or specificity determining residues described above. Thus, provided in one embodiment is a humanized monoclonal antibody comprising an antigen-binding portion binding CD9 and an antigen-binding portion binding another antigen such as CD137, HVEM or CD7, wherein each antigen-binding portion comprises a variable domain comprising human acceptor framework regions and non-human donor CDRs.

Examples of human frameworks which can be used in the invention are KOL, NEWM, REI, EU, TUR, TEI, LAY and POM (Kabat et al, supra). For example, KOL and NEWM can be used for the heavy chain, REI can be used for the light chain and EU, LAY and POM can be used for both the heavy chain and the light chain. Alternatively, human germline sequences may be used; these are available at, for example: http://vbase.mrc-cpe.cam.ac.uk/. In a CDR-grafted antibody of the invention, the acceptor heavy and light chains do not necessarily need to be derived from the same antibody and may, if desired, comprise composite chains having framework regions derived from different chains.

Fully human antibodies are those antibodies in which the variable regions and the constant regions (where present) of both the heavy and the light chains are all of human origin, or substantially identical to sequences of human origin, not necessarily from the same antibody.

Examples of fully human antibodies may include antibodies produced for example by the phage display methods described above and antibodies produced by mice in which the murine immunoglobulin variable and constant region genes have been replaced by their human counterparts, e.g., as described in general terms in EP0546073, U.S. Pat. Nos. 5,545,806, 5,569,825, 5,625,126, 5,633,425, 5,661,016, 5,770,429, EP 0438474 and EP0463151.

Furthermore, the antibody of the invention may comprise a heavy chain constant region selected from an IgG1, an IgG2, an IgG3 or an IgG4 isotype, or a variant thereof. The constant region domains of the antibody of the invention, if present, may be selected having regard to the proposed function of the antibody, and in particular the effector functions which may be required. For example, the human IgG constant region domains of the IgG1 and IgG3 isotypes may be used when the antibody effector functions are required. Alternatively, IgG2 and IgG4 isotypes may be used when the antibody effector functions are not required. For example, IgG4 molecules in which the serine at position 241 has been changed to proline as described in Angal et al., Molecular Immunology, 1993, 30 (1), 105-108 may be used. Particularly preferred is the IgG4 constant domain that comprises this change.

It should also be appreciated that antigen-binding portions comprised in the antibody of the invention such as the functionally-active fragments or derivatives of a whole immunoglobulin fragments described above, may be incorporated into other antibody formats than being the antigen-binding portions of the classic IgG format. Alternative format to the classic IgG may include those known in the art and those described herein, such as DVD-Igs, FabFvs for example as disclosed in WO2009/040562 and WO2010/035012, diabodies, triabodies, tetrabodies etc. Other examples include a diabody, triabody, tetrabody, bibodies and tribodies (see for example Holliger and Hudson, 2005, Nature Biotech 23(9): 1 126-1136; Schoonjans et al. 2001 , Biomolecular Engineering, 17 (6), 193-202), tandem scFv, tandem scFv-Fc, FabFv, Fab′Fv, FabdsFv, Fab-scFv, Fab′-scFv, diFab, diFab′, scdiabody, scdiabody-Fc, ScFv-Fc-scFv, scdiabody-CH3, IgG-scFv, scFv-IgG, V-IgG, IgG-V, DVD-Ig, DuoBody, Fab-Fv-Fv, Fab-Fv-Fc and Fab-dsFv-PEG fragments described in WO2009040562, WO2010035012, WO2011/08609, WO2011/030107 and WO201 1/061492, respectively.

In one embodiment of the present invention, there is provided an antibody, preferably a monoclonal antibody, comprising a first antigen-binding portion binding CD9 and a second antigen-binding portion binding another antigen, such as antigen expressed on T cells, wherein the antibody further comprises at least an additional antigen-binding portion. In further variants of this embodiment, the second antigen binding portion may bind an antigen secreted by T cell or another immune system cell such as B cells or NK cells or an antigen expressed on a cell such as an immune system cell.

In one embodiment, the antibody comprises a first antigen-binding portion binding CD9 and a second antigen-binding portion binding CTLA4 and an additional antigen-binding portion binding for example PD1 or PDL1. More specifically, the second antigen-binding portion binding CTLA4 may be the antigen-binding portion of ipilimumab or of tremelimumab and the additional antigen-binding portion binding PD1 or PDL1 may be the antigen-binding portion of pembrolizumab or of nivolumab or of cemiplimab (PD1) or the antigen-binding portion of atezolizumab or of avelumab or of durvalumab (PDL1).

Furthermore, the antibody of the invention may comprise along with the first antigen-binding portions binding CD9 and the second antigen-binding portion binding another antigen such as CD137, HVEM or CD7, also at least an additional antigen-binding portion.

In one embodiment, the additional antigen-binding portion may bind yet another antigen expressed or secreted by T cells such as CD137, HVEM or CD7.

In another embodiment, there is provided an antibody, preferably a monoclonal antibody, comprising a first antigen-binding portion binding CD9 and a second antigen-binding portion binding CD137, wherein the antibody is humanised and wherein the antibody further comprises an additional antigen-binding portion. More preferably this antibody is capable of activating T cells and/or enhancing induction of T cell proliferation. The stimulation may be an anti-CD3 stimulation wherein activating T cell may result in an upregulation of CD71, CD25 and CD137. The additional antigen-binding portion may for example bind HVEM or CD7.

In another embodiment, there is provided an antibody, preferably a monoclonal antibody, comprising a first antigen-binding portion binding CD9 and a second antigen-binding portion binding HVEM, wherein the antibody is humanised and wherein the antibody further comprises an additional antigen-binding portion. More preferably this antibody is capable of activating T cells and/or enhancing induction of T cell proliferation. The stimulation may be SEB stimulation wherein activating T cells may result in an upregulation of CD137, CD71 and CD25. The additional antigen-binding portion may for example bind CD137 or CD7.

In another embodiment, there is provided an antibody, preferably a monoclonal antibody, comprising a first antigen-binding portion binding CD7 and a second antigen-binding portion binding CD9, wherein the antibody is humanised and wherein the antibody further comprises an additional antigen-binding portion. More preferably this antibody is capable of upregulating or enhancing cytokine production and/or enhancing T cell proliferation and/or enhancing induction of T cell proliferation. The stimulation may be an anti-CD3 stimulation wherein upregulating or enhancing cytokine production results in an upregulation of granzyme B and/or IFNgamma. The additional antigen-binding portion may for example bind HVEM or CD137.

In preferred embodiments, the additional antigen-binding portion is capable of increasing, i.e. extending, the half-life of the antibody. Preferably, the additional antigen-binding portion binds albumin, more preferably human serum albumin.

The antibody of the present invention may be comprised in a pharmaceutical composition along with one or more pharmaceutically acceptable excipients. By pharmaceutical composition is intended a composition for both therapeutic and diagnostic use. In another aspect, the present invention provides for a pharmaceutical composition comprising an antibody, preferably a monoclonal antibody, comprising a first antigen-binding portion binding CD9 and a second antigen-binding portion binding another antigen such as antigen expressed on T cells, wherein the antibody is preferably humanised and wherein the composition comprises one or more pharmaceutically acceptable excipients. In further variants of this embodiment, the second antigen binding portion may bind an antigen secreted by T cell or another immune system cell such as B cells or NK cells or an antigen expressed on a cell such as an immune system cell.

In one embodiment, the pharmaceutical composition comprises an antibody comprising a first antigen-binding portion binding CD9 and a second antigen-binding portion binding CTLA4. For example, the second antigen-binding portion binding CTLA4 may be the antigen-binding portion of ipilimumab or of tremelimumab.

In another embodiment, the pharmaceutical composition comprises an antibody comprising a first antigen-binding portion binding CD9 and a second antigen-binding portion binding PD1. For example, the second antigen-binding portion may be the antigen-binding portion of pembrolizumab or of nivolumab or of cemiplimab.

In another embodiment, the pharmaceutical composition comprises an antibody comprising a first antigen-binding portion binding CD9 and a second antigen-binding portion binding PD1L1. For example, the second antigen-binding portion may be the antigen-binding portion of atezolizumab or of avelumab or of durvalumab.

In one preferred embodiment, the antibody comprises a first antigen-binding portion binding an extracellular domain region of human CD9, wherein the extracellular domain region of CD9 is preferably loop 2 of CD9, and wherein more preferably the first antigen-binding portion binds within amino acids 112 to 195 of SEQ ID NO: 1 and a second antigen-binding portion binding human CD137 as defined in SEQ ID NO: 2 or from amino acid 24 to 186 of SEQ ID NO: 2, wherein the first and the second antigen-binding portions are located on the same antibody, i.e. they are part of the same polypeptide chain, associate via one or more non-covalent and/or covalent associations or linked so as to form one single molecule.

In another preferred embodiment, the antibody comprises a first antigen-binding portion binding an extracellular domain region of human CD9, wherein the extracellular domain region of CD9 is preferably loop 2 of CD9, and wherein more preferably the first antigen-binding portion binds within amino acids 112 to 195 of SEQ ID NO: 1 and a second antigen-binding portion binding the HVEM as defined in SEQ ID NO: 3 or from amino acid 39 to 283 of SEQ ID NO: 3 or from amino acid 39 to 202 of SEQ ID NO: 3, wherein the first and the second antigen-binding portions are located on the same antibody, i.e. they are part of the same polypeptide chain, associate via one or more non-covalent and/or covalent associations or linked so as to form one single molecule.

In one preferred embodiment, the antibody comprises a first antigen-binding portion binding an extracellular domain region of human CD9, wherein the extracellular domain region of CD9 is preferably loop 2 of CD9, and wherein more preferably the first antigen-binding portion binds within amino acids 112 to 195 of SEQ ID NO: 1 and a second antigen-binding portion binding CD7 as defined in SEQ ID NO: 4 or from amino acid 26 to 240 of SEQ ID NO: 4 or from or from amino acid 26 to 180 of SEQ ID NO:4, wherein the first and the second antigen-binding portions are located on the same antibody, i.e. they are part of the same polypeptide chain, associate via one or more non-covalent and/or covalent associations or linked so as to form one single molecule.

In another embodiment, the first antigen-binding portion binding CD9 of the antibody of the present invention comprises a first heavy chain variable region and a first light chain variable region and the second antigen-binding portion comprises a second heavy chain variable region and a second light chain variable region and binds CD137 and wherein:
a. The first heavy chain variable region comprises a CDR-H1 comprising SEQ ID NO: 11, a CDR-H2 comprising SEQ ID NO: 12 and a CDR-H3 comprising SEQ ID NO: 13; and
b. The first light chain variable region comprises a CDR-L1 comprising SEQ ID NO: 14, a CDR-L2 comprising SEQ ID NO: 15 and a CDR-L3 comprising SEQ ID NO: 16; and
c. The second heavy chain variable region comprises a CDR-H1 comprising SEQ ID NO: 5, a CDR-H2 comprising SEQ ID NO: 6 and a CDR-H3 comprising SEQ ID NO: 7; and
d. The second light chain variable region comprises a CDR-L1 comprising SEQ ID NO: 8, a CDR-L2 comprising SEQ ID NO: 9 and a CDR-L3 comprising SEQ ID NO: 10;
or
e. The first heavy chain variable region comprises SEQ ID NO: 21 and the first light chain variable region comprises SEQ ID NO: 23; and the second heavy chain variable region comprises SEQ ID NO: 17 and second light chain variable region comprises SEQ ID NO: 19;
or
f. The first heavy chain variable region is encoded by a nucleotide sequence comprising SEQ ID NO: 22 and the first light chain variable region is encoded by a nucleotide sequence comprising SEQ ID NO: 24; and the second heavy chain variable region is encoded by a nucleotide sequence comprising SEQ ID NO: 18 and second light chain variable region is encoded by a nucleotide sequence comprising SEQ ID NO: 20.

In one embodiment, the first antigen-binding portion binding CD9 of the antibody of the present invention comprises a first heavy chain variable region and a first light chain variable region and the second antigen-binding portion comprises a second heavy chain variable region and a second light chain variable region and binds HVEM and wherein:
a. The first heavy chain variable region comprises a CDR-H1 comprising SEQ ID NO: 11, a CDR-H2 comprising SEQ ID NO: 12 and a CDR-H3 comprising SEQ ID NO: 13; and
b. The first light chain variable region comprises a CDR-L1 comprising SEQ ID NO: 14, a CDR-L2 comprising SEQ ID NO: 15 and a CDR-L3 comprising SEQ ID NO: 16; and
c. The second heavy chain variable region comprises a CDR-H1 comprising SEQ ID NO: 25, a CDR-H2 comprising SEQ ID NO: 26 and a CDR-H3 comprising SEQ ID NO: 27; and
d. The second light chain variable region comprises a CDR-L1 comprising SEQ ID NO: 30;
or
e. The first heavy chain variable region comprises SEQ ID NO: 21 and the first light chain variable region comprises SEQ ID NO: 23; and the second heavy chain variable region comprises SEQ ID NO: 31 and second light chain variable region comprises SEQ ID NO: 33;
or
f. The first heavy chain variable region is encoded by a nucleotide sequence comprising SEQ ID NO: 22 and the first light chain variable region is encoded by a nucleotide sequence comprising SEQ ID NO: 24; and the second heavy chain variable region is encoded by a nucleotide sequence comprising SEQ ID NO: 32 and second light chain variable region is encoded by a nucleotide sequence comprising SEQ ID NO: 34.

In one embodiment, the first antigen-binding portion binding CD9 of the antibody of the present invention comprises a first heavy chain variable region and a first light chain variable region and the second antigen-binding portion comprises a second heavy chain variable region and a second light chain variable region and binds CD7 and wherein:
g. The first heavy chain variable region comprises a CDR-H1 comprising SEQ ID NO: 11, a CDR-H2 comprising SEQ ID NO: 12 and a CDR-H3 comprising SEQ ID NO: 13; and
h. The first light chain variable region comprises a CDR-L1 comprising SEQ ID NO: 14, a CDR-L2 comprising SEQ ID NO: 15 and a CDR-L3 comprising SEQ ID NO: 16; and
i. The second heavy chain variable region comprises a CDR-H1 comprising SEQ ID NO: 35, a CDR-H2 comprising SEQ ID NO: 36 and a CDR-H3 comprising SEQ ID NO: 37; and
j. The second light chain variable region comprises a CDR-L1 comprising SEQ ID NO: 38, a CDR-L2 comprising SEQ ID NO: 39 and a CDR-L3 comprising SEQ ID NO: 40;
or
k. The first heavy chain variable region comprises SEQ ID NO: 21 and the first light chain variable region comprises SEQ ID NO: 23; and the second heavy chain variable region comprises SEQ ID NO: 41 and second light chain variable region comprises SEQ ID NO: 43;
or
l. The first heavy chain variable region is encoded by a nucleotide sequence comprising SEQ ID NO: 22 and the first light chain variable region is encoded by a nucleotide sequence comprising SEQ ID NO: 24; and the second heavy chain variable region is encoded by a nucleotide sequence comprising SEQ ID NO: 42 and second light chain variable region is encoded by a nucleotide sequence comprising SEQ ID NO: 44.

In one embodiment, the antibody according to the present invention is prepared according to the disclosure of WO2015/181282, WO2016/009030, WO2016/009029, WO2017/093402, WO2017/093404 and WO2017/093406, which are all incorporated herein by reference.

More specifically, the antibody is made by the heterodimerization of a Fab-X and a Fab-Y.

Fab-X comprises a Fab fragment which comprises the first antigen-binding portion binding CD9 which comprises a first heavy chain variable region and a first light chain variable region wherein the first heavy chain variable region comprises a CDR-H1 comprising SEQ ID NO: 11, a CDR-H2 comprising SEQ ID NO: 12 and a CDR-H3 comprising SEQ ID NO: 13; and the first light chain variable region comprises a CDR-L1 comprising SEQ ID NO: 14, a CDR-L2 comprising SEQ ID NO: 15 and a CDR-L3 comprising SEQ ID NO: 16. The Fab comprising the first antigen-binding portion binding CD9 is linked to a scFv (clone 52SR4), preferably via a peptide linker to the C-terminal of the CH1 domain of the Fab fragment and the VL domain of the scFv. The scFv may itself also contains a peptide linker located in between its VL and VH domains.

Fab-Y also comprises a Fab fragment which comprises the second antigen-binding portion. The second antigen-binding portion comprises a second heavy chain variable region and a second light chain variable region.

In one embodiment second antigen-binding portion binds to:
a) CD137 and the second heavy chain variable region comprises a CDR-H1 comprising SEQ ID NO: 5, a CDR-H2 comprising SEQ ID NO: 6 and a CDR-H3 comprising SEQ ID NO: 7; and the second light chain variable region comprises a CDR-L1 comprising SEQ ID NO: 8, a CDR-L2 comprising SEQ ID NO: 9 and a CDR-L3 comprising SEQ ID NO: 10; or
b) HVEM and second heavy chain variable region comprises a CDR-H1 comprising SEQ ID NO: 25, a CDR-H2 comprising SEQ ID NO: 26 and a CDR-H3 comprising SEQ ID NO: 27; and the second light chain variable region comprises a CDR-L1 comprising SEQ ID NO: 30; or
c) CD7 and the second heavy chain variable region comprises a CDR-H1 comprising SEQ ID NO: 35, a CDR-H2 comprising SEQ ID NO: 36 and a CDR-H3 comprising SEQ ID NO: 37; and the second light chain variable region comprises a CDR-L1 comprising SEQ ID NO: 38, a CDR-L2 comprising SEQ ID NO: 39 and a CDR-L3 comprising SEQ ID NO: 40.

The Fab comprising the second antigen-binding portion is preferably linked to a peptide GCN4 (clone 7P14P), preferably via a peptide linker to the CH1 domain of the Fab fragment.

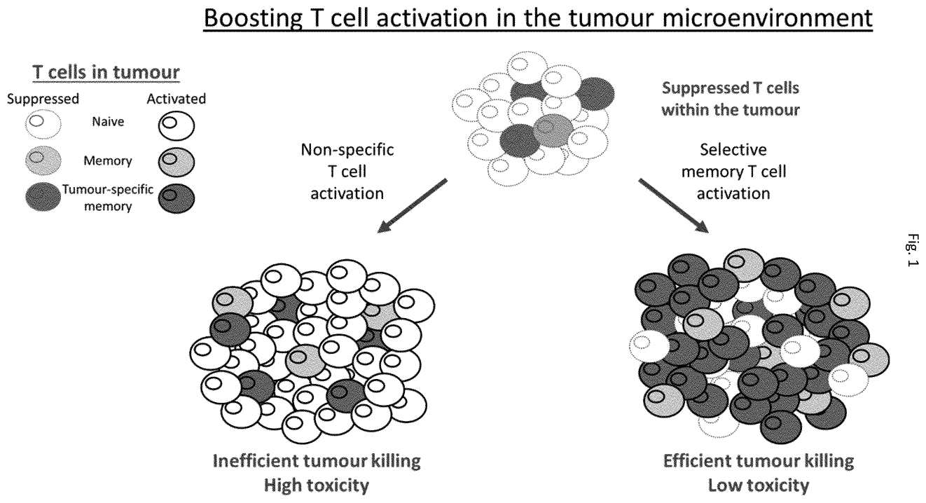

The scFv of Fab-X is specific for and complementary to the peptide GCN4 of Fab-Y. As a result, when the Fab-X and the Fab-Y are brought into contact with each other, a non-covalent binding interaction between the scFv and GCN4 peptide occurs, thereby physically retaining the two antigen-binding portions in the form of a complex resulting in an antibody comprising two antigen-binding portions on the same molecule (FIG. 1).

In another embodiment, the Fab-X comprises the first antigen-binding portion binding CD9 which comprises a first heavy chain variable region comprises SEQ ID NO: 21 and the first light chain variable region comprises SEQ ID NO: 23; and Fab-Y comprises the second antigen-binding portion binding to:
a) CD137 which second antigen-binding portion comprises the second heavy chain variable region which comprising SEQ ID NO: 17 and second light chain variable region which comprising SEQ ID NO: 19; or
b) HVEM which second antigen-binding portion comprises the second heavy chain variable region comprising SEQ ID NO: 31 and second light chain variable region comprising SEQ ID NO: 33; or
c) CD7 which second antigen-binding portion comprises the second heavy chain variable region comprising SEQ ID NO: 41 and second light chain variable region comprising SEQ ID NO: 43

Binding specificities may be exchanged between Fab-X and Fab-Y, i.e. in one embodiment Fab-X may comprise the antigen-binding portion binding to CD9 and Fab-Y the antigen-binding portion binding to another antigen such as CD137, HVEM, CD7 etc; in another embodiment, Fab-Y may comprise the antigen-binding portion binding to CD9 and Fab-X the antigen-binding portion binding to another antigen such as CD137, HVEM, CD7 etc.

In another embodiment, the pharmaceutical composition comprises an antibody, preferably a monoclonal antibody, which antibody comprises a first antigen-binding portion binding CD9 and a second antigen-binding portion binding another antigen such as CD137, HVEM or CD7, wherein the antibody is preferably humanised and wherein the composition comprises one or more pharmaceutically acceptable excipients.

Pharmaceutically acceptable excipients in therapeutic compositions may additionally contain liquids such as water, saline, glycerol and ethanol. Additionally, auxiliary substances, such as wetting or emulsifying agents or pH buffering substances, may be present in such compositions. Such excipients enable the pharmaceutical compositions to be formulated as tablets, pills, dragees, capsules, liquids, gels, syrups, slurries and suspensions, for ingestion by the patient.

The antibody of the present invention and the pharmaceutical composition comprising such antibody may be used in therapy.

Therefore, in another aspect, the present invention provides for an antibody, preferably a monoclonal antibody, or a pharmaceutical composition comprising the antibody, and one or more pharmaceutically acceptable excipients, wherein the antibody comprises a first antigen-binding portion binding CD9 and a second antigen-binding portion binding another antigen such as antigen expressed on T cells, wherein the antibody is preferably humanised and is for use in therapy. In further embodiments of this aspect, the second antigen binding portion may bind an antigen secreted by T cell or another immune system cell such as B cells or NK cells or an antigen expressed on a cell such as an immune system cell.

In one embodiment, the antibody comprising a first antigen-binding portion binding CD9 and a second antigen-binding portion binding CTLA4, such as the antigen-binding portion of ipilimumab or of tremelimumab or a pharmaceutical composition comprising this antibody is for use in therapy.

In another embodiment, the antibody comprising a first antigen-binding portion binding CD9 and a second antigen-binding portion binding PD1, such as the antigen-binding portion of pembrolizumab or of nivolumab or of cemiplimab or a pharmaceutical composition comprising this antibody is for use in therapy.

In another embodiment, the antibody comprising a first antigen-binding portion binding CD9 and a second antigen-binding portion binding PD1L1, such as the antigen-binding portion of atezolizumab or of avelumab or of durvalumab or a pharmaceutical composition comprising this antibody is for use in therapy.

In a preferred embodiment, the antibody, or a pharmaceutical composition comprising the antibody, and one or more pharmaceutically acceptable excipients, is for use in therapy, wherein the antibody comprises a first antigen-binding portion binding CD9 and a second antigen-binding portion binding another antigen such as CD137, HVEM or CD9, wherein the antibody is preferably humanised.

In another aspect, the present invention provides for an antibody, preferably a monoclonal antibody, or a pharmaceutical composition comprising the antibody, and one or more pharmaceutically acceptable excipients, wherein the antibody comprises a first antigen-binding portion binding CD9 and a second antigen-binding portion binding another antigen such as CTLA4, PD1, PDL1, CD137, HVEM or CD7, wherein the antibody is preferably humanised and is for use in the treatment of cancer and/or an infectious disease.

In one embodiment, the antibody or composition comprising such antibody for use in therapy an antibody comprising a first antigen-binding portion binding an extracellular domain region of human CD9, wherein the extracellular domain region of CD9 is preferably loop 2 of CD9, and wherein more preferably the first antigen-binding portion binds within amino acids 112 to 195 of SEQ ID NO: 1 and a second antigen-binding portion binding human CD137 as defined in SEQ ID NO: 2 or from amino acid 24 to 186 of SEQ ID NO: 2, wherein the first and the second antigen-binding portions are located on the same antibody, i.e. they are part of the same polypeptide chain, associate via one or more non-covalent and/or covalent associations or linked so as to form one single molecule.

In another embodiment, the antibody or composition comprising such antibody for use in therapy is an antibody comprising a first antigen-binding portion binding an extracellular domain region of human CD9, wherein the extracellular domain region of CD9 is preferably loop 2 of CD9, and wherein more preferably the first antigen-binding portion binds within amino acids 112 to 195 of SEQ ID NO: 1 and a second antigen-binding portion binding the HVEM as defined in SEQ ID NO: 3 or from amino acid 39 to 283 of SEQ ID NO: 3 or from amino acid 39 to 202 of SEQ ID NO: 3, wherein the first and the second antigen-binding portions are located on the same antibody, i.e. they are part of the same polypeptide chain, associate via one or more non-covalent and/or covalent associations or linked so as to form one single molecule.

In yet another embodiment, the antibody or composition comprising such antibody for use in therapy is an antibody comprising a first antigen-binding portion binding an extracellular domain region of human CD9, wherein the extracellular domain region of CD9 is preferably loop 2 of CD9, and wherein more preferably the first antigen-binding portion binds within amino acids 112 to 195 of SEQ ID NO: 1 and a second antigen-binding portion binding CD7 as defined in SEQ ID NO: 4 or from amino acid 26 to 240 of SEQ ID NO: 4 or from or from amino acid 26 to 180 of SEQ ID NO:4, wherein the first and the second antigen-binding portions are located on the same antibody, i.e. they are part of the same polypeptide chain, associate via one or more non-covalent and/or covalent associations or linked so as to form one single molecule.

In one embodiment, the antibody comprising a first antigen-binding portion binding CD9 and a second antigen-binding portion binding CTLA4, such as the antigen-binding portion of ipilimumab or of tremelimumab or a pharmaceutical composition comprising this antibody is for use in the treatment of cancer and/or an infectious disease.

In another embodiment, the antibody comprising a first antigen-binding portion binding CD9 and a second antigen-binding portion binding PD1, such as the antigen-binding portion of pembrolizumab or of nivolumab or of cemiplimab or a pharmaceutical composition comprising this antibody is for use in the treatment of cancer and/or an infectious disease.

In another embodiment, the antibody comprising a first antigen-binding portion binding CD9 and a second antigen-binding portion binding PD1L1, such as the antigen-binding portion of atezolizumab or of avelumab or of durvalumab or a pharmaceutical composition comprising this antibody is for use in the treatment of cancer and/or an infectious disease.

In yet another aspect, the present invention provides for a method for treating a subject afflicted with cancer and/or an infectious disease, comprising administering to the subject a pharmaceutically effective amount of an antibody, preferably a monoclonal antibody, or a pharmaceutical composition comprising the antibody, and one or more pharmaceutically acceptable excipients, wherein the antibody comprises a first antigen-binding portion binding CD9 and a second antigen-binding portion binding another antigen such as an antigen expressed on T cells, in particular CTLA4, PD1, PDL1, CD137, HVEM or CD9, wherein the antibody is preferably humanised.

In another embodiment, the antibody or composition comprising such antibody for use in the treatment of cancer and/or an infectious disease is an antibody comprising a first antigen-binding portion binding an extracellular domain region of human CD9, wherein the extracellular domain region of CD9 is preferably loop 2 of CD9, and wherein more preferably the first antigen-binding portion binds within amino acids 112 to 195 of SEQ ID NO: 1 and a second antigen-binding portion binding human CD137 as defined in SEQ ID NO: 2 or from amino acid 24 to 186 of SEQ ID NO: 2, wherein the first and the second antigen-binding portions are located on the same antibody, i.e. they are part of the same polypeptide chain, associate via one or more non-covalent and/or covalent associations or linked so as to form one single molecule.

In another embodiment, the antibody or composition comprising such antibody for use in the treatment of cancer and/or an infectious disease is an antibody comprising a first antigen-binding portion binding an extracellular domain region of human CD9, wherein the extracellular domain region of CD9 is preferably loop 2 of CD9, and wherein more preferably the first antigen-binding portion binds within amino acids 112 to 195 of SEQ ID NO: 1 and a second antigen-binding portion binding the HVEM as defined in SEQ ID NO: 3 or from amino acid 39 to 283 of SEQ ID NO: 3 or from amino acid 39 to 202 of SEQ ID NO: 3, wherein the first and the second antigen-binding portions are located on the same antibody, i.e. they are part of the same polypeptide chain, associate via one or more non-covalent and/or covalent associations or linked so as to form one single molecule.

In another embodiment, the antibody or composition comprising such antibody for use in the treatment of cancer and/or an infectious disease is an antibody comprising a first antigen-binding portion binding an extracellular domain region of human CD9, wherein the extracellular domain region of CD9 is preferably loop 2 of CD9, and wherein more preferably the first antigen-binding portion binds within amino acids 112 to 195 of SEQ ID NO: 1 and a second antigen-binding portion binding CD7 as defined in SEQ ID NO: 4 or from amino acid 26 to 240 of SEQ ID NO: 4 or from or from amino acid 26 to 180 of SEQ ID NO:4, wherein the first and the second antigen-binding portions are located on the same antibody, i.e. they are part of the same polypeptide chain, associate via one or more non-covalent and/or covalent associations or linked so as to form one single molecule.

In one embodiment, the method for treating a subject afflicted with cancer and/or an infectious disease comprises administering to a subject (such as a human subject) a pharmaceutically effective amount of an antibody, or a pharmaceutical composition comprising the antibody and one or more pharmaceutically acceptable excipients, wherein the antibody comprises a first antigen-binding portion binding CD9 and a second antigen-binding portion binding CTLA4, such as the antigen-binding portion of ipilimumab or of tremelimumab.

In another embodiment, the method for treating a subject afflicted with cancer and/or an infectious disease comprises administering to a subject (such as a human subject) a pharmaceutically effective amount of an antibody, or a pharmaceutical composition comprising the antibody and one or more pharmaceutically acceptable excipients, wherein the antibody comprises a first antigen-binding portion binding CD9 and a second antigen-binding portion binding PD1, such as the antigen-binding portion of pembrolizumab or of nivolumab or of cemiplimab.

In another embodiment, the method for treating a subject afflicted with cancer and/or an infectious disease comprises administering to a subject (such as a human subject) a pharmaceutically effective amount of an antibody, or a pharmaceutical composition comprising the antibody and one or more pharmaceutically acceptable excipients, wherein the antibody comprises a first antigen-binding portion binding CD9 and a second antigen-binding portion binding PDL1, such as the antigen-binding portion of atezolizumab or of avelumab or of durvalumab or a pharmaceutical composition comprising this antibody is for use in the treatment of cancer and/or an infectious disease.

The subjects to be treated is preferably a human subject. In one embodiment, there is provided for a method for treating a human subject afflicted with cancer and/or an infectious disease, comprising administering to the subject a pharmaceutically effective amount of an antibody, preferably a monoclonal antibody, or a pharmaceutical composition comprising the antibody and one or more pharmaceutically acceptable excipients, wherein the antibody comprises a first antigen-binding portion binding CD9 and a second antigen-binding portion binding another antigen, such as an antigen expressed on T cells, in particular CD137, HVEM or CD9, wherein the antibody is preferably humanised.

In yet another embodiment, the method for treating a human subject afflicted with cancer and/or an infectious disease comprises administering an antibody or composition comprising such antibody comprising a first antigen-binding portion binding an extracellular domain region of human CD9, wherein the extracellular domain region of CD9 is preferably loop 2 of CD9, and wherein more preferably the first antigen-binding portion binds within amino acids 112 to 195 of SEQ ID NO: 1 and a second antigen-binding portion binding human CD137 as defined in SEQ ID NO: 2 or from amino acid 24 to 186 of SEQ ID NO: 2, wherein the first and the second antigen-binding portions are located on the same antibody, i.e. they are part of the same polypeptide chain, associate via one or more non-covalent and/or covalent associations or linked so as to form one single molecule.

In yet another embodiment, the method for treating a human subject afflicted with cancer and/or an infectious disease comprises administering an antibody or composition comprising such antibody comprising a first antigen-binding portion binding an extracellular domain region of human CD9, wherein the extracellular domain region of CD9 is preferably loop 2 of CD9, and wherein more preferably the first antigen-binding portion binds within amino acids 112 to 195 of SEQ ID NO: 1 and a second antigen-binding portion binding the HVEM as defined in SEQ ID NO: 3 or from amino acid 39 to 283 of SEQ ID NO: 3 or from amino acid 39 to 202 of SEQ ID NO: 3, wherein the first and the second antigen-binding portions are located on the same antibody, i.e. they are part of the same polypeptide chain, associate via one or more non-covalent and/or covalent associations or linked so as to form one single molecule.

In yet another embodiment, the method for treating a human subject afflicted with cancer and/or an infectious disease comprises administering to the human subject an antibody or composition comprising such antibody comprising a first antigen-binding portion binding an extracellular domain region of human CD9, wherein the extracellular domain region of CD9 is preferably loop 2 of CD9, and wherein more preferably the first antigen-binding portion binds within amino acids 112 to 195 of SEQ ID NO: 1 and a second antigen-binding portion binding CD7 as defined in SEQ ID NO: 4 or from amino acid 26 to 240 of SEQ ID NO: 4 or from or from amino acid 26 to 180 of SEQ ID NO:4, wherein the first and the second antigen-binding portions are located on the same antibody, i.e. they are part of the same polypeptide chain, associate via one or more non-covalent and/or covalent associations or linked so as to form one single molecule.

In another aspect of the present invention, there is provided the use of an antibody, preferably a monoclonal antibody, or a pharmaceutical composition comprising the antibody, and one or more pharmaceutically acceptable excipients, wherein the antibody comprises a first antigen-binding portion binding CD9 and a second antigen-binding portion binding another antigen, such as an antigen expressed on T cells, in particular CTLA4, PD1, PDL1, CD137, HVEM or CD9, wherein the antibody is preferably humanised, in the manufacture of a medicament for treating cancer and/or an infectious disease.

In one embodiment, the use of an antibody comprising a first antigen-binding portion binding CD9 and a second antigen-binding portion binding CTLA4, such as the antigen-binding portion of ipilimumab or of tremelimumab or a pharmaceutical composition comprising this antibody is for the manufacture of a medicament for treating cancer and/or an infectious disease.

In another embodiment, the use of an antibody comprising a first antigen-binding portion binding CD9 and a second antigen-binding portion binding PD1, such as the antigen-binding portion of pembrolizumab or of nivolumab or of cemiplimab or a pharmaceutical composition comprising this antibody is for the manufacture of a medicament for treating cancer and/or an infectious disease.

In another embodiment, the use of an antibody comprising a first antigen-binding portion binding CD9 and a second antigen-binding portion binding PD1L1, such as the antigen-binding portion of atezolizumab or of avelumab or of durvalumab or a pharmaceutical composition comprising this antibody is for the manufacture of a medicament for treating cancer and/or an infectious disease.

Example of cancers that may be treated using the antibody, or pharmaceutical composition comprising such antibody, include but are not limited to, Acute Lymphoblastic Leukaemia, Acute Myeloid Leukaemia, Adrenocortical Carcinoma, AIDS-Related Cancers Kaposi Sarcoma (Soft Tissue Sarcoma), AIDS-Related Lymphoma, Primary CNS Lymphoma, Anal Cancer, Appendix Cancer Astrocytomas, Atypical Teratoid/Rhabdoid Tumour, Brain Cancer, Basal Cell Carcinoma of the Skin, Bile Duct Cancer, Bladder Cancer, Bone Cancer (includes Ewing Sarcoma and Osteosarcoma and Malignant Fibrous Histiocytoma), Breast Cancer, Bronchial Tumours, Burkitt Lymphoma, Carcinoid Tumour, Cardiac (Heart) Tumours, Embryonal Tumours, Germ Cell Tumour, Primary CNS Lymphoma, Cervical Cancer, Cholangiocarcinoma, Chordoma, Chronic Lymphocytic Leukaemia, Chronic Myelogenous Leukaemia, Chronic Myeloproliferative Neoplasms, Colorectal Cancer, Craniopharyngioma, Cutaneous T-Cell Lymphoma, Ductal Carcinoma In Situ, Endometrial Cancer (Uterine Cancer), Ependymoma, Oesophageal Cancer, Esthesioneuroblastoma (Head and Neck Cancer), Ewing Sarcoma (Bone Cancer), Extracranial Germ Cell Tumour, Extragonadal Germ Cell Tumour, Eye Cancer, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumour, Gastrointestinal Stromal Tumours (Soft Tissue Sarcoma), Ovarian Germ Cell Tumours, Testicular Cancer, Gestational Trophoblastic Disease, Hairy Cell Leukaemia, Head and Neck Cancer, Hepatocellular (Liver) Cancer, Histiocytosis, Langerhans Cell Hodgkin Lymphoma, Hypopharyngeal Cancer, Intraocular Melanoma, Islet Cell Tumours, Pancreatic Neuroendocrine Tumours, Kaposi Sarcoma (Soft Tissue Sarcoma), Kidney (Renal Cell) Cancer, Langerhans Cell Histiocytosis, Laryngeal Cancer, Leukaemia, Lip and Oral Cavity Cancer, Liver Cancer, Lung Cancer (Non-Small Cell and Small Cell), Male Breast Cancer, Malignant Fibrous Histiocytoma of Bone and Osteosarcoma, Melanoma, Intraocular (Eye) Childhood Intraocular, Merkel Cell Carcinoma, Mesothelioma, Metastatic Cancer, Metastatic Squamous Neck Cancer with Occult Primary, Midline Tract Carcinoma With NUT Gene Changes, Mouth Cancer, Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukaemia, Chronic, Myeloid Leukaemia, Acute, Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Lip and Oral Cavity Cancer and Oropharyngeal Cancer, Osteosarcoma and Malignant Fibrous Histiocytoma of Bone, Ovarian Cancer, Pancreatic Cancer, Pancreatic Neuroendocrine Tumours (Islet Cell Tumours), Papillomatosis, Paraganglioma, Paranasal Sinus and Nasal Cavity Cancer, Parathyroid Cancer, Penile Cancer, Pharyngeal Cancer, Pheochromocytoma, Pituitary Tumour, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Salivary Gland Cancer, Sarcoma Childhood Rhabdomyosarcoma, Childhood Vascular Tumours, Ewing Sarcoma, Kaposi Sarcoma, Osteosarcoma, Soft Tissue Sarcoma, Uterine Sarcoma, Sézary Syndrome, Skin Cancer, Small Intestine Cancer, Squamous Cell Carcinoma of the Skin, Squamous Neck Cancer with Occult Primary, Stomach (Gastric) Cancer, Cutaneous T-Cell Lymphoma, Testicular Cancer, Throat Cancer, Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter, Urethral Cancer, Uterine Cancer, Endometrial Uterine Sarcoma, Vaginal Cancer, Vascular Tumours, Vulvar Cancer and Wilms Tumour, and any combinations of these cancers. The present invention is also applicable to treatment of metastatic cancers.

The antibody according to the present invention, or the pharmaceutical composition comprising such antibody, may be administered concomitantly or sequentially to one or more additional cancer therapies. By cancer therapies is intended drug-based therapies as well as other type of cancer therapies such as radiotherapies.

The invention will now be further described by way of examples with references to embodiments illustrated in the accompanying drawings

### EXAMPLES

### Example 1: T Cell Activation Primary Screen

The activation status of T cells can be assessed through their expression of cell surface markers and secreted cytokines which play important roles in cellular function. Activated T cells for example upregulate CD25, CD71 and CD137 on their surface and produce increased amounts of granzyme B, IFNgamma and IL-2, essential mediators to effect killing of virus-infected or tumour cell targets. T cells in the tumour microenvironment are often maintained in a suppressed state and express only low levels of these proteins. Agents that overcome this suppression and induce T cell activation and proliferation have tremendous therapeutic potential as they may unleash effective anti-tumour T cell responses and promote cancer elimination.

To identify novel modulators of T cell activation, a large screen was undertaken whereby 49 antigen specificities were combined to generate a grid of bispecific antibodies with a theoretical size of 1,176 possible bispecific combinations. The specificities were selected from the literature as being expressed on T cells or expressed on other cell types involved in interactions with T cells. Of this potential grid, 969 novel antigen bispecific constructs were tested, covering 82.4% of the possible combinations. Between 1 and 4 different antibodies were tested for each specificity, and all were tested on peripheral blood mononuclear cells (PBMC) from donors in combination with a negative control arm to identify the effect of the monovalent forms of the construct.

PBMC represent the major leukocyte classes involved in both innate and adaptive immunity, apart from granulocytes. PBMC comprise a heterogenous population of cells which when manipulated in vitro provide a relatively more relevant physiological environment compared to isolated component cell types such as T cells and monocytes, that are no longer capable of responding to paracrine and autocrine signals provided by other cells. As such identification of molecules modulating specific subsets of cells within the wider PBMC population, have increased translational potential to more complex biological systems, ultimately increasing success rates for modulating immune cell interactions in disease.

Each bispecific combination was tested on two PBMC donors. This negative control arm is a Fab from an antibody raised to an antigen not expressed on PBMC.

Fusion proteins were prepared according to the disclosure of WO2015/181282, WO2016/009030, WO2016/009029, WO2017/093402, WO2017/093404 and WO2017/093406, which are all incorporated herein by reference. The first fusion protein (A-X) includes a Fab fragment (A of the A-X) with specificity to one antigen, which is linked to X, a scFv (clone 52SR4) via a peptide linker to the C-terminal of the CH1 domain of the Fab fragment and the VL domain of the scFv. The scFv itself also contains a peptide linker located in between its VL and VH domains.

The second fusion protein (B-Y) includes a Fab fragment (B of the B-Y) with specificity to another antigen. However, in comparison to the first protein, the Fab fragment B is attached to Y, a peptide GCN4 (clone 7P14P) via a peptide linker to the CH1 domain of the Fab fragment.

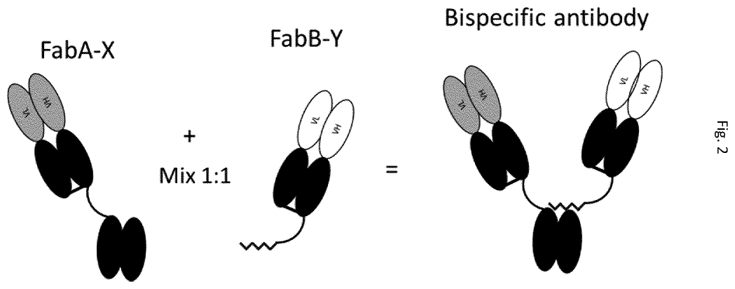

The scFv, X, is specific for and complementary to the peptide GCN4, Y. As a result, when the two fusion proteins are brought into contact with each other, a non-covalent binding interaction between the scFv and GCN4 peptide occurs, thereby physically retaining the two fusion proteins in the form of a complex mimicking an antibody comprising two antigen-binding portions on the same molecule (FIG. 2).

Purified Fab-X and Fab-Y with varying specificities were incubated together for 60 minutes (in a 37° C./5% CO₂ environment) at an equimolar concentration. The final molarity of each tested complex was 100 nM. In 384-well tissue culture plates, 1×10⁵ PBMC were added to wells, to which were added pre-formed Fab-X/Fab-Y bispecific antibodies. Following the bispecific antibodies addition, the cells were incubated for 48 hours at 37° C./5% CO₂, with or without 250 ng/mL (final concentration) anti-human CD3 antibody (clone UCHT1) or with or without 1 μg/mL (final concentration) Staphylococcal Enterotoxin-B (SEB) superantigen (SAg). After 48 hours the plates were centrifuged at 500×g for 5 minutes at 4° C. Cell culture conditioned media were transferred from the cell pellets to fresh plates and frozen at −80° C. Cells were washed with 60 μL FACS buffer two times by centrifugation, followed by resuspension of the pellets by shaking the plates at 2200 rpm for 30 seconds. The cells were stained with a cocktail of fluorescently labeled antibodies as listed in the Table 2 and incubated at 4° C. in the dark for 1 hour.

TABLE 2

| Epitope | Fluorophore | Clone | Source | Dilution |
|---|---|---|---|---|
| CD56 | APC | HCD56 | BioLegend ® | 40 |
| CD14 | PE/Cy7 | M5E2 | BD Pharmagen ® | 80 |
| CD8 | BV650 | RPA-T8 | BioLegend ® | 80 |
| CD4 | BV605 | RPA-T4 | BD Horizon ® | 40 |
| CD19 | PE-Cy5 | HIB19 | BD Pharmagen ® | 20 |
| CD45RO | BV786 | UCHL1 | BD Horizon ® | 40 |
| CD71 | FITC | M-A712 | BD Pharmagen ® | 20 |
| CD25 | BV510 | M-A251 | BD Horizon ® | 40 |
| CD137 | PE | 4B4-1 | BD Pharmagen ® | 20 |
| Live/Dead | Near IR | | Thermo Scientific ® | 1000 |

After this time, cells were washed twice with FACS buffer, before fixing with 2% paraformaldehyde (BD CellFix™, diluted in dH₂O) and stored overnight at 4° C. The plates were centrifuged at 500×g for 5 minutes, the fixation buffer aspirated to waste and the cells resuspended in a residual volume of 10 μL for acquisition on the iQue® Screener Plus (IntelliCyr). The data analysis software package Forecyt™ (IntelliCyt®) was used to gate on CD14+ monocytes, CD56+ NK cells, CD19+ B cells, CD4+ and CD8+ memory and naïve T cells. For each cell population the cellular expression of CD25, CD71 and CD137 was measured as reported median fluorescent intensity values. The data were then used to calculate the log 2 fold changes of expression relative to control well values.

The resulting flow cytometry data were analysed by identification of the CD4 and CD8 memory and naïve T cells and well as the B cells, NK cells and monocytes. The median fluorescence intensities (MFIs) of the 4 activation markers CD71, CD25 and CD137 were then determined and the log 2 fold changes calculated relative to the control wells for each cell population. These results were deposited into the visualisation software, Spotfire®, where it was possible to identify antigen pairs capable of inducing an increase in T cell activation.

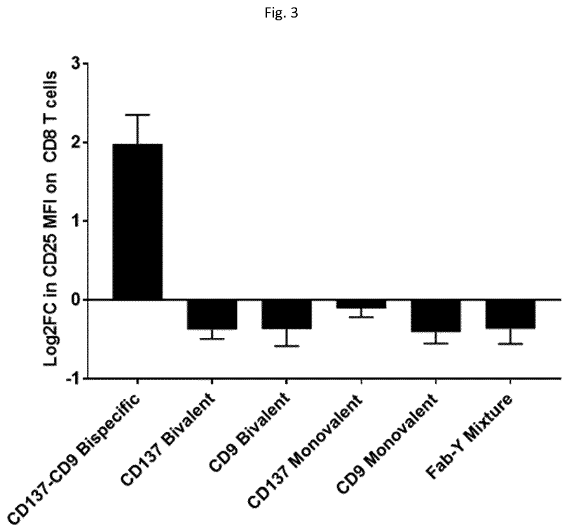

In addition to the cell analysis, the levels of granzyme B or IFNgamma in unstimulated or anti-CD3 or SAg SEB stimulated conditions were then studied. The supernatants from the untreated samples were thawed and diluted 40-fold before analysis of the level of granzyme B (FIG. 2) or IFNgamma (FIG. 3), measured to create untreated baselines. The PBMC culture supernatants treated with the antibodies were thawed and diluted 20-fold for the anti-CD3 and SAg SEB stimulated plates, and 5-fold for the unstimulated plates. The diluted conditioned media was then assayed for levels of the proteins granzyme B and IFNgamma using an IntelliCyt® QBead PlexScreen.

Various phenotypes such as the enhancement of activated T cells through inhibition of T cell expressed markers, upregulation of cytokine release and T cell proliferation were investigated herein in order to reflect different biological mechanisms important for the therapy of cancer and infectious diseases.

When considering antigen pairs capable of upregulating the later activation markers CD25, CD71 and CD137 on T cells in the presence of an anti-CD3 stimulation or SEB SAg stimulation, but not in unstimulated conditions, pairs of targets comprising CD9 and another antigen, in the present examples CD137, HVEM, respectively, were identified.

When considering antigen pairs capable of upregulating the levels of granzyme B and IFNgamma in the supernatant in conjunction with SAg and anti-CD3, CD9/CD7 combinations were identified.

Bispecific antibodies binding CD9 and another antigen, in this case, CD9/CD137, CD9/HVEM and CD9/CD7 were therefore taken into subsequent assays to show that their effect was repeatable across a larger number of donors.

### Example 2: Follow Up Assays

To confirm the effect of anti-CD9 bispecific antibodies on stimulation of T cells through inhibition of T cell expressed markers, additional PBMC donors were assayed in stimulated and unstimulated conditions.

A grid of fusion proteins Fab-X and Fab-Y were created by diluting equimolar (1 μM) quantities of Fab-X (Fab-scFv) and Fab-Y (Fab-peptide) with specificity for CD9 and CD137 or CD9 and HVEM or CD9 and CD7 in TexMACS™ media (Miltenyi Biotec®) containing 100 U/mL penicillin/100 μg/mL streptomycin. Mixtures of equimolar (1 μM) Fab-Y proteins were also generated in the same manner. The Fab-X and Fab-Y fusion proteins were incubated together for 1 hour (in a 37° C./5% CO₂ environment), at a final concentration of 500 nM. Negative control wells contained TexMACS™ media only were also generated alongside the Fab-X and Fab-Y wells.

During this time, cryopreserved human PBMC isolated from platelet leukapheresis cones were thawed and washed in TexMACs™ media and resuspended at 3.33×10⁶ cells/mL. The PBMC were then seeded into 384-well flat bottom tissue culture plates (Greiner Bio-one®) at 30 μL/well (1×10⁵ PBMC). A total of 10 μL of Fab-X/Fab-Y bispecific antibodies were transferred to the plates containing 30 μL PBMC. The PBMC were then either left unstimulated by the addition of 10 μL of TexMACS™ media, or stimulated with 10 μL of either soluble anti-CD3 (UCHT1) (250 or 10 ng/mL final concentration) or SAg SEB (1 μg/mL final concentration). This resulted in a final assay concentration of Fab-X/Fab-Y bispecific antibodies of 100 nM. The plates were then returned to a 37° C./5% CO₂ environment for 48 hours.

After 48 hours the plates were centrifuged at 500×g for 5 minutes at 4° C. Cell culture conditioned media were transferred from the cell pellets to fresh plates and frozen at −80° C.

For cell markers, cells were washed with 60 μL FACS buffer two times by centrifugation, followed by resuspension of the pellets by shaking the plates at 2200 rpm for 30 seconds. The cells were stained with a cocktail of fluorescently labeled antibodies as listed in Table 1 and incubated at 4° C. in the dark for 1 hour. After this time, cells were washed twice with FACS buffer, before fixing with 2% paraformaldehyde (BD CellFix™, diluted in dH₂O) and stored overnight at 4° C. The plates were centrifuged at 500×g for 5 minutes, the fixation buffer aspirated to waste and the cells resuspended in a residual volume of 10 μL for acquisition on the iQue® Screener Plus (IntelliCyt®).

The data analysis software package Forecyt™ (IntelliCyt®) was used to gate on CD14+ monocytes, CD56+ NK cells, CD19+ B cells, CD4+ and CD8+ memory and naïve T cells. For each cell population the cellular expression of CD25, CD71 and CD137 was measured as reported median fluorescent intensity values. The data were then used to calculate the log 2 fold changes of expression relative to control well values.

For cytokine release, on the day of analysis, the conditioned media was thawed and diluted 40-fold in RPMI cell culture medium (ThermoFisher). The diluted conditioned media was then assayed for levels of granzyme B and IFNgamma using an IntelliCyt® QBead PlexScreen. Standards curves of known protein concentrations were generated alongside, allowing for the calculation of the absolute concentrations for these proteins in the supernatant.

The data analysis software package Forecyt™ (IntelliCyt®) was used to measure the median fluorescent intensity values for the granzyme B and IFNgamma detection beads. The data were then used to generate standard curves and calculate the concentrations. The log 2 fold changes of granzyme B and IFNgamma concentrations were calculated relative to control well values.

CD9-CD137 Bispecific Antibodies

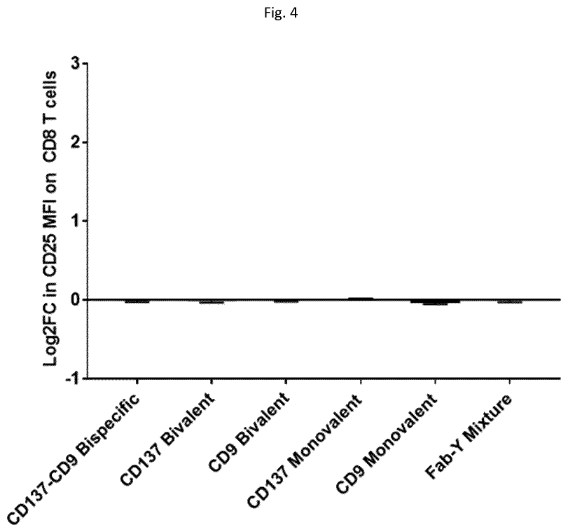

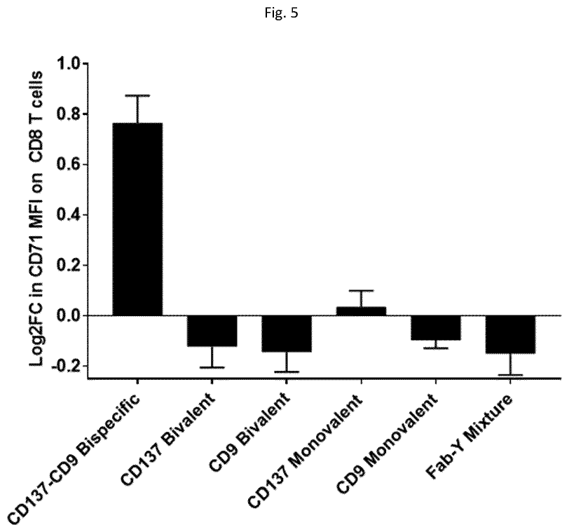

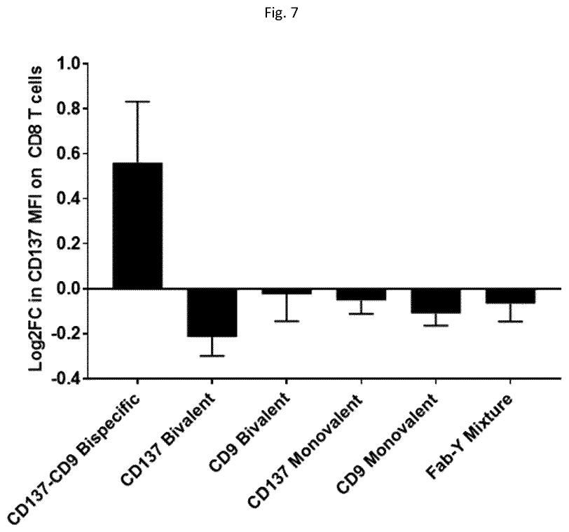

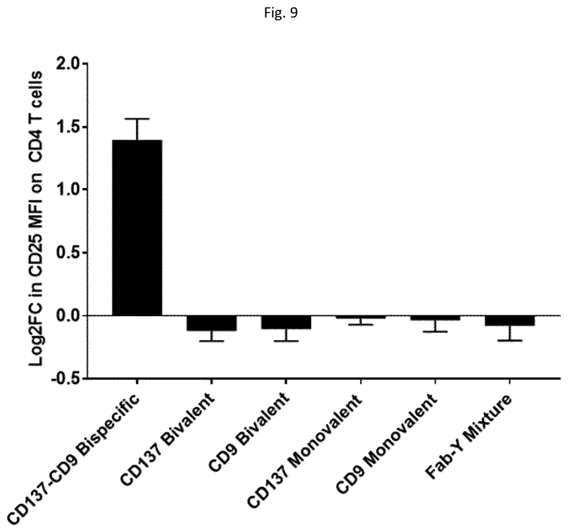

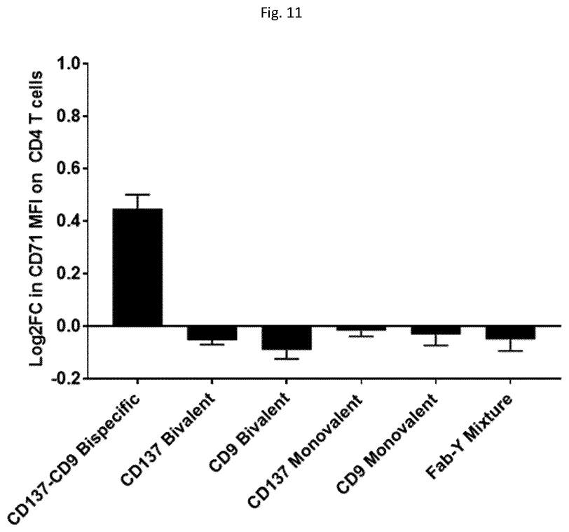

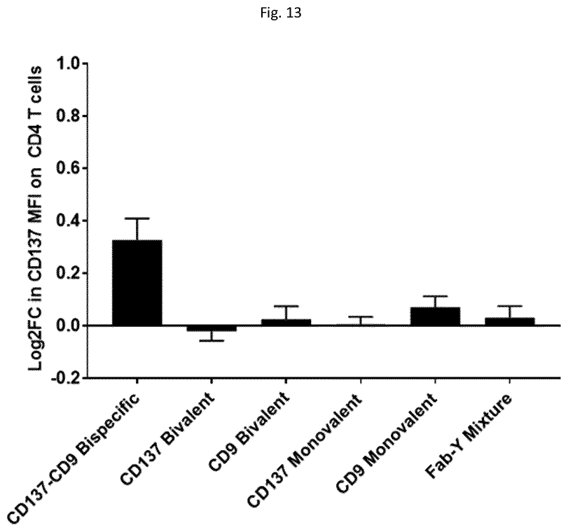

Seven CD9-CD137 bispecific antibodies showed increased expression of activation markers CD71, CD25 and CD137 on CD4+ and CD8+ T cells in combination with anti-CD3. FIGS. 4 to 15 show an example bispecific combination as well as the bivalent, monovalent and Fab-Y mixture controls. The CD137/CD9 bispecific antibody is shown to increase the expression of all three activation markers on CD8+ T cells (FIG. 3: CD25; FIG. 5: CD71; FIG. 7: CD137) and on CD4+ T cells (FIG. 9: CD25; FIG. 11: CD71; FIG. 13: CD137) when added to PBMCs stimulated with soluble anti-CD3 for 48 hours. The bivalents antibodies, formed by fusions where both Fab in the Fab-X/Fab-Y antibody are specific for either CD137 or CD9 (as the case may be) and monovalent antibodies for CD137 and CD9, formed by fusions where the Fab in the Fab-X is specific for CD137 or CD9 but the Fab in the Fab-Y is a negative control (in the present case the Fab of an anti-idiotypic antibody, hence not for an antigen expressed on or by any cell) did not lead to a similar increase in the activation marker fluorescence intensity on either cell populations. This confirms that neither the binding of CD137 alone or CD9 alone can stimulate activation in the absence of the other. Furthermore, the mixture of Fab-Y binding CD137 and Fab-Y binding CD9 also had no stimulatory effect on the T cell populations. This further confirms that there is a requirement, for the stimulatory function to occur, for the antigen-binding portions binding CD137 and CD9 to be on the same molecule, or on separate molecules which become associated via non-covalent or covalent associations or linkers.

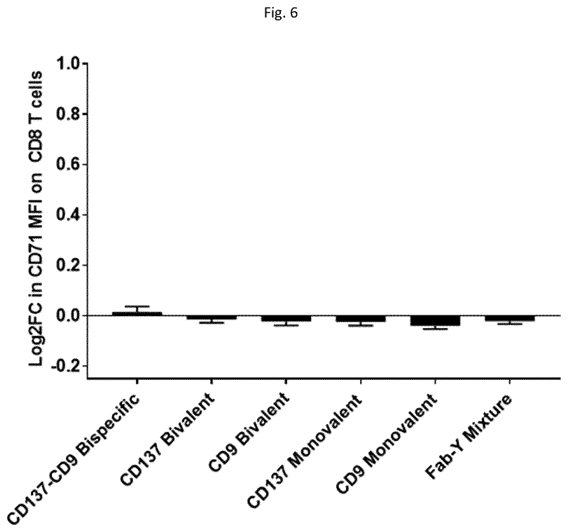

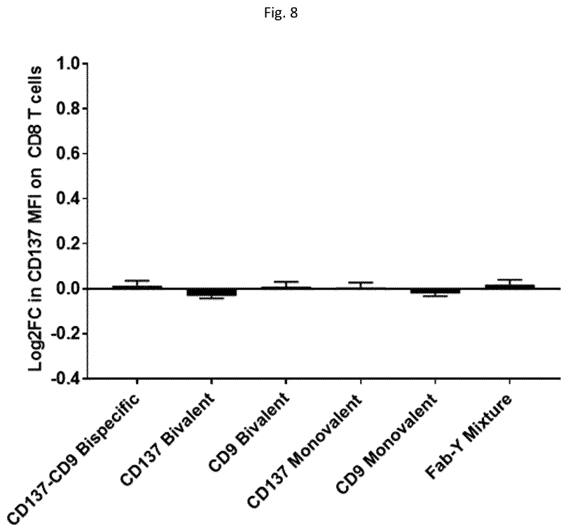

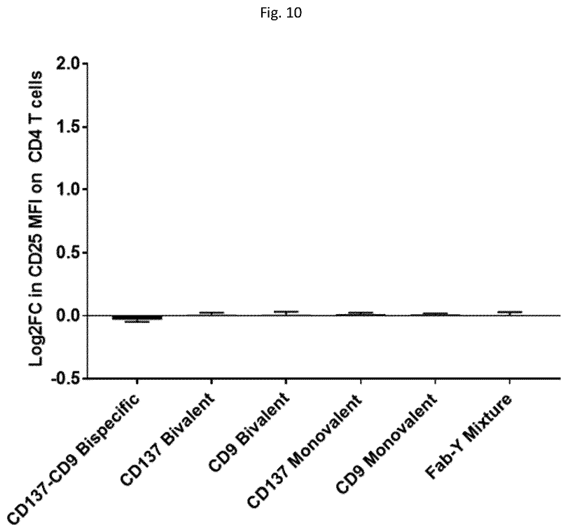

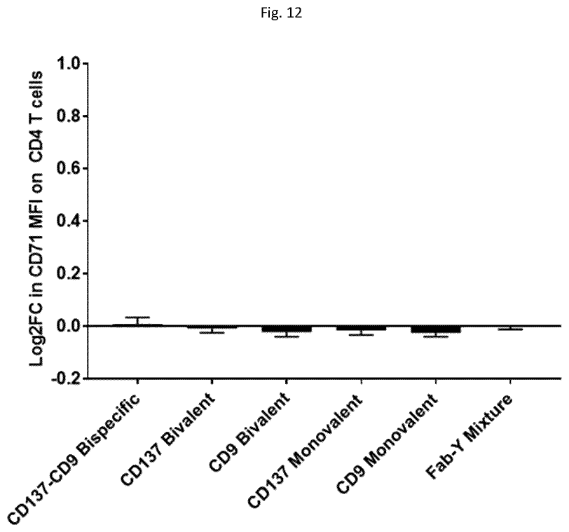

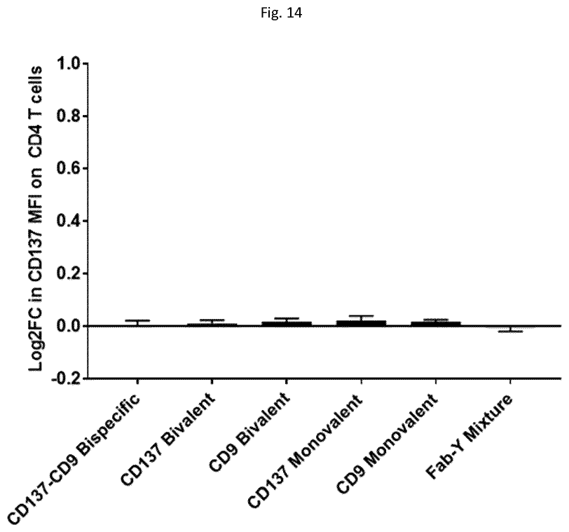

When studied in unstimulated conditions the CD137-CD9 bispecific antibodies did not lead to any increases in activation marker fluorescence intensity in either CD8+ T cells (FIG. 4: CD25; FIG. 6: CD71; FIG. 8: CD137) or CD4+ T cells (FIG. 10: CD25; FIG. 12: CD71; FIG. 14: CD137). This confirms that the binding of both CD137 and CD9 does not lead to the unwanted activation of resting T cells.

The statistical significance of the log 2 fold changes in activation markers of the CD137-CD9 antibodies, relative to the bivalent, monovalent and mixture controls, in stimulated or unstimulated conditions, is shown in Table 3.

TABLE 3

| Activation marker | | |
|---|---|---|
| CD25 | CD71 | CD137 |

CD8 T cells in anti-CD3

| | | | |
|---|---|---|---|
| CD137-CD9 Bispecific vs. CD137 Bivalent | <0.0001 | <0.0001 | 0.001 |
| CD137-CD9 Bispecific vs. CD9 Bivalent | <0.0001 | <0.0001 | 0.018 |
| CD137-CD9 Bispecific vs. CD137 | <0.0001 | <0.0001 | 0.0124 |
| Monovalent | | | |
| CD137-CD9 Bispecific vs. CD9 | <0.0001 | <0.0001 | 0.0051 |
| Monovalent | | | |
| CD137-CD9 Bispecific vs. Fab-Y Mixture | <0.0001 | <0.0001 | 0.0099 |

CD8 T cells in Unstimulated

| | | | |
|---|---|---|---|
| CD137-CD9 Bispecific vs. CD137 Bivalent | 0.9972 | 0.6715 | 0.4274 |
| CD137-CD9 Bispecific vs. CD9 Bivalent | >0.9999 | 0.4582 | >0.9999 |
| CD137-CD9 Bispecific vs. CD137 | 0.9988 | 0.3708 | 0.9997 |
| Monovalent | | | |
| CD137-CD9 Bispecific vs. CD9 | 0.6073 | 0.1 | 0.7016 |
| Monovalent | | | |
| CD137-CD9 Bispecific vs. Fab-Y Mixture | >0.9999 | 0.4876 | >0.9999 |

CD4 T cells in anti-CD3

| | | | |
|---|---|---|---|
| CD137-CD9 Bispecific vs. CD137 Bivalent | <0.0001 | <0.0001 | <0.0001 |
| CD137-CD9 Bispecific vs. CD9 Bivalent | <0.0001 | <0.0001 | 0.0005 |
| CD137-CD9 Bispecific vs. CD137 | <0.0001 | <0.0001 | 0.0002 |
| Monovalent | | | |
| CD137-CD9 Bispecific vs. CD9 | <0.0001 | <0.0001 | 0.0036 |
| Monovalent | | | |
| CD137-CD9 Bispecific vs. Fab-Y Mixture | <0.0001 | <0.0001 | 0.0006 |

CD4 T cells in Unstimulated

| | | | |
|---|---|---|---|
| CD137-CD9 Bispecific vs. CD137 Bivalent | 0.5777 | 0.975 | 0.9998 |
| CD137-CD9 Bispecific vs. CD9 Bivalent | 0.5576 | 0.7361 | 0.9816 |
| CD137-CD9 Bispecific vs. CD137 | 0.4615 | 0.8693 | 0.9424 |
| Monovalent | | | |
| CD137-CD9 Bispecific vs. CD9 | 0.5395 | 0.6266 | 0.9812 |
| Monovalent | | | |
| CD137-CD9 Bispecific vs. Fab-Y Mixture | 0.7487 | 0.9966 | 0.9898 |

Statistical analysis was performed in GraphPad Prism®, and a One-way ANOVA was used with multiple comparisons (Sidak's Multiple comparison test). Values presented represent the p values calculated from the Sidak's multiple comparison test; n=4 donors, 2 technical replicates.

CD9-HVEM Bispecific Antibodies

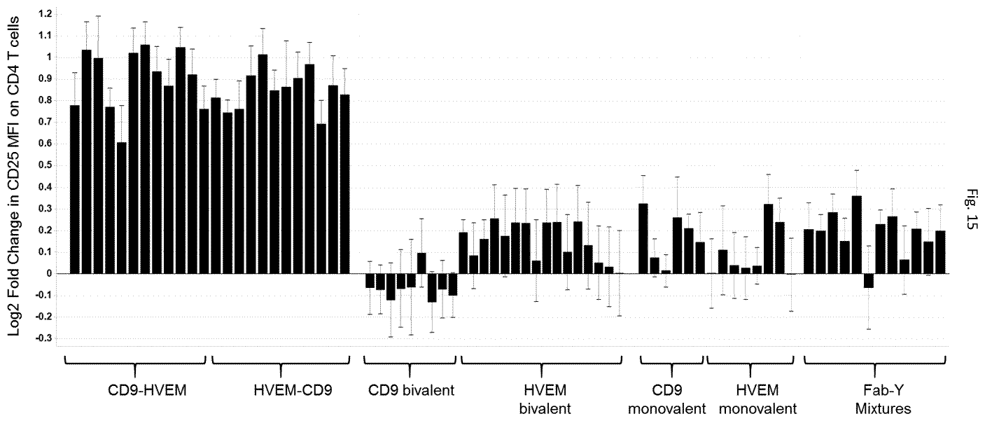

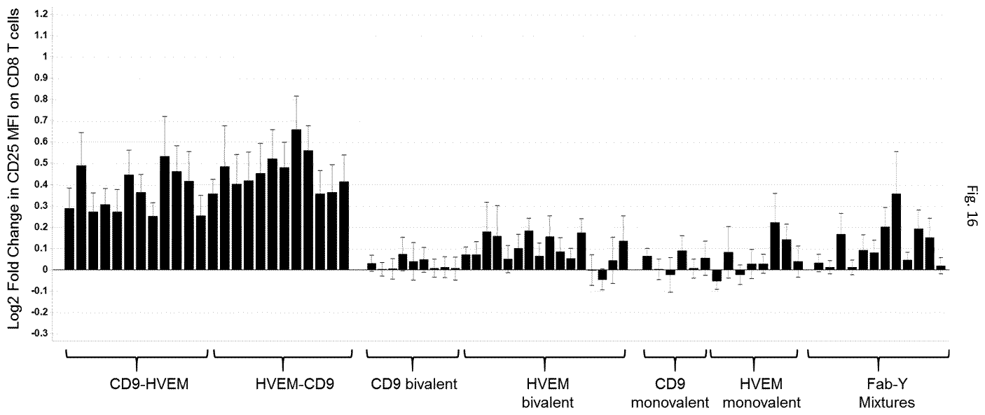

Twenty-four CD9-HVEM bispecific antibodies showed increased expression of the activation marker CD25 on CD4+ (FIG. 15) and CD8+ (FIG. 16) T cells in combination with SEB, while the control constructs did not lead to this increase. The bivalents (i.e. formed by a fusion where both Fab in the Fab-X and Fab-Y are specific for CD9 or HVEM as the case may be) and monovalent antibodies for CD9 or HVEM (i.e. formed by a fusion where the Fab is specific for CD9 or HVEM but the other component Fab is a negative control) did not lead to a similar increase in the activation marker fluorescence intensity on either cell populations, suggesting that a monospecific antibody binding CD9 alone or HVEM alone, whether in a monovalent or bivalent fashion, is unable to stimulate activation in the absence of the binding to the other antigen. The small increases in CD25 expression detected by the HVEM bivalent and monovalent controls in CD4+ T cells, however were only small increases, which could only be greatly enhanced by the coupling with CD9. Furthermore, the mixture of an antibody binding solely to HVEM and an antibody binding solely to CD9 (Fab-Y mixture) also had no enhanced stimulatory effect on the T cell populations compared to the HVEM controls, implying the requirement for the antigen-binding portions binding HVEM and CD9 to be on the same chain, associated via non-covalent associations or linked for the stimulatory function to occur.

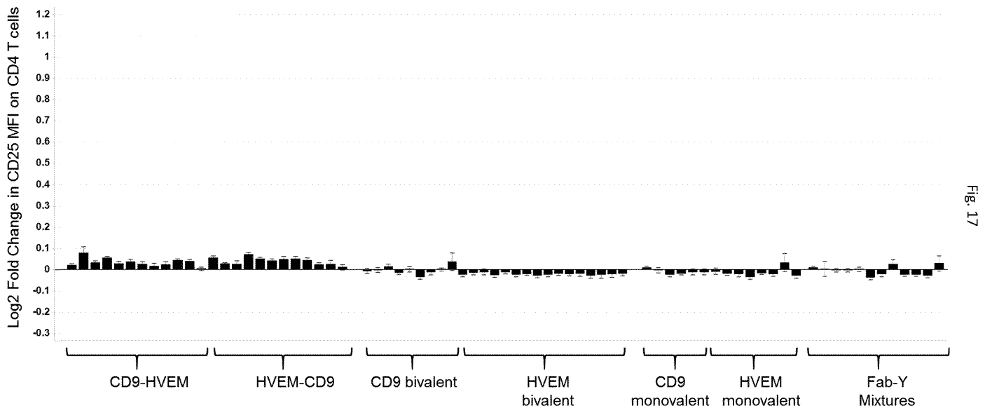

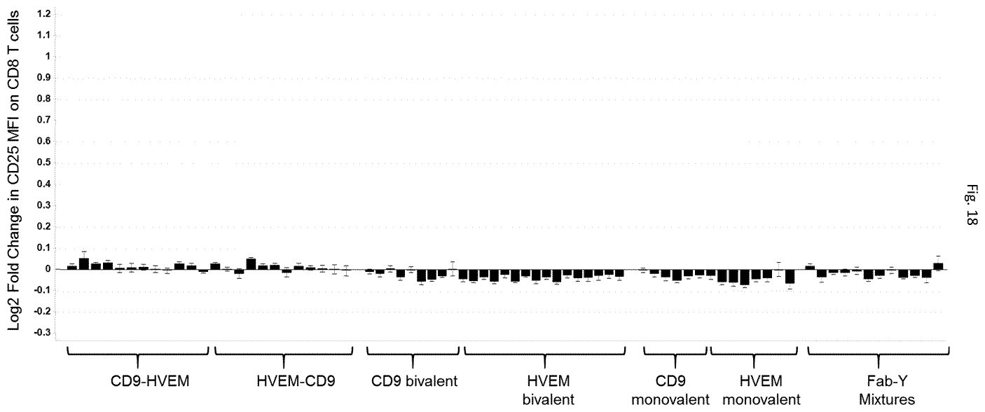

When studied in unstimulated conditions the HVEM-CD9 bispecific antibodies did not lead to any increases in activation marker fluorescence intensity in either CD4+ T cells (FIG. 17) or CD8+ T cells (FIG. 18). This confirms that the binding of both HVEM and CD9 does not lead to the unwanted activation of resting T cells.

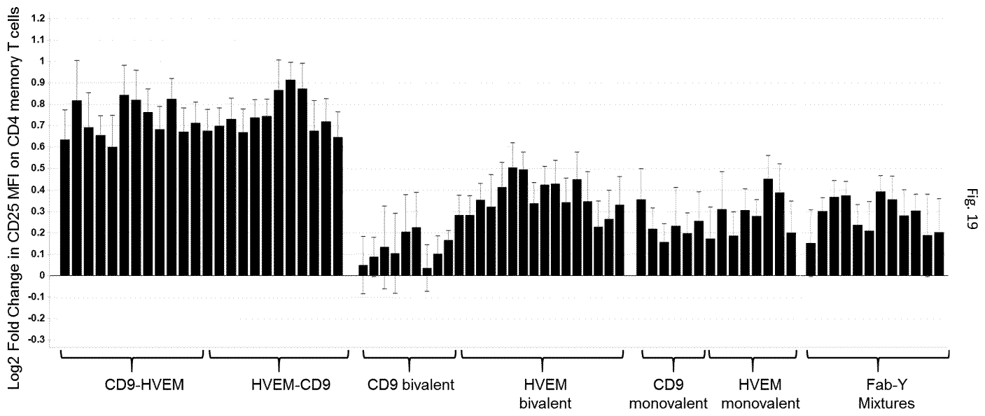

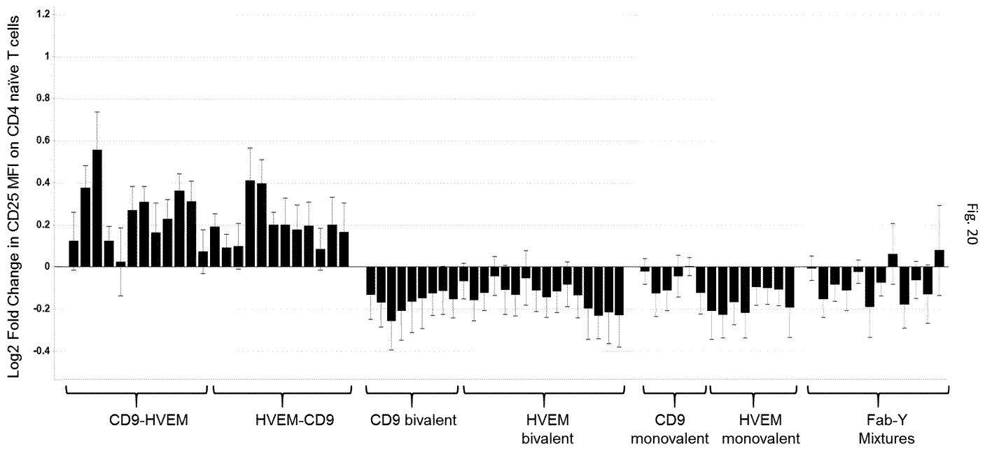

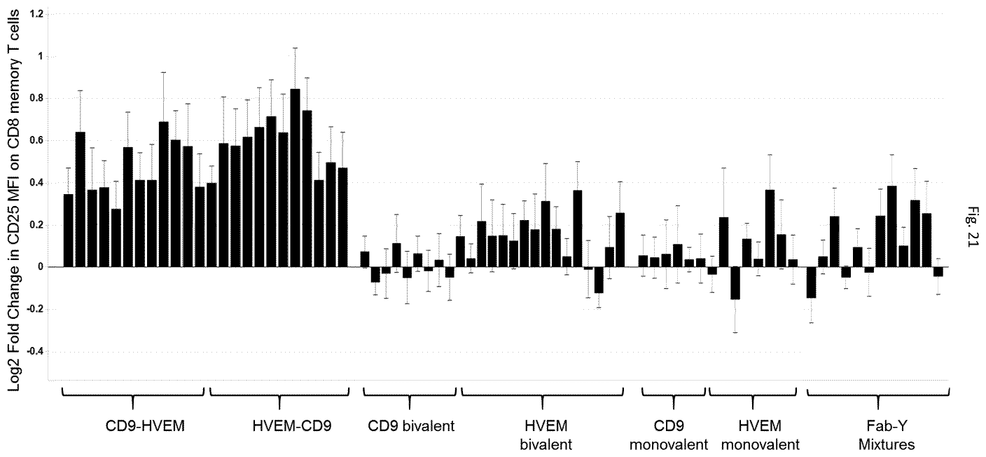

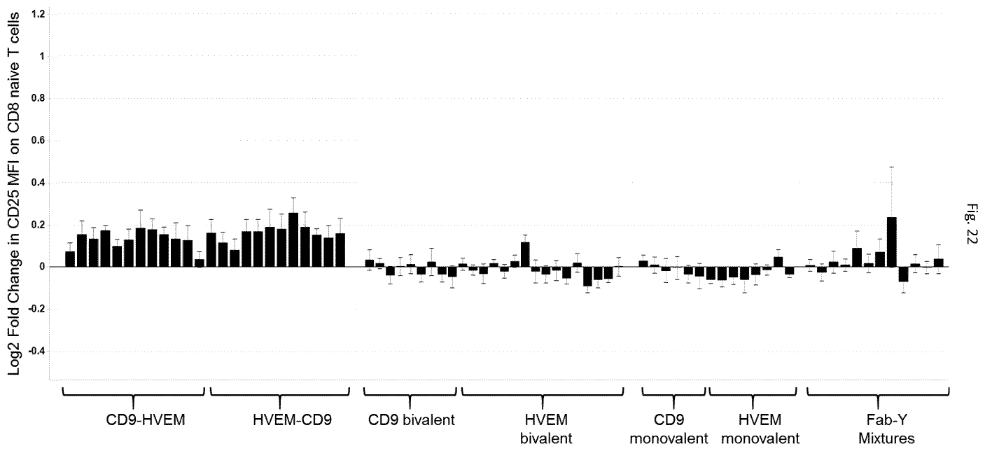

Furthermore, it was possible to measure the level of CD25 expression on naïve versus memory T cell populations by the separation of the CD45RO expressing memory cells from the CD45RO non-expressing naïve T cell populations. Upon analysis of these sub populations a preference for upregulation of CD25 on the memory CD4+ and CD8+ T cell populations compared to the naïve populations was identified (FIGS. 19-22). This suggests that HVEM-CD9 bispecific antibodies could preferentially target the memory T cell populations, enhancing their therapeutic potential.

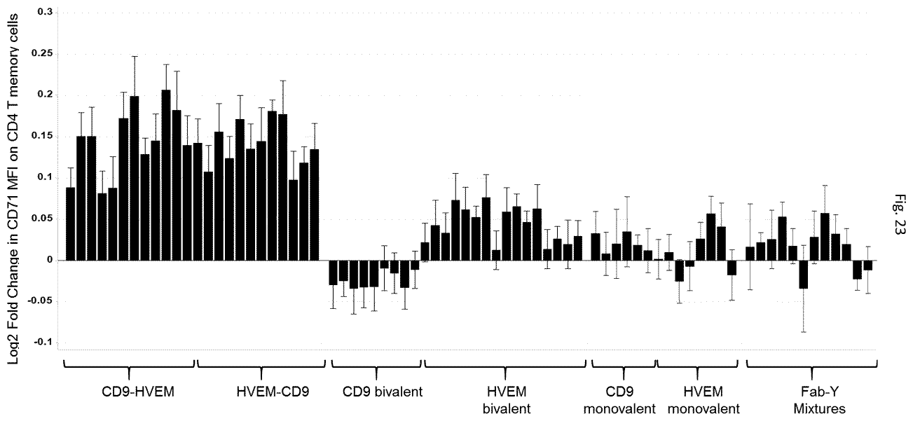

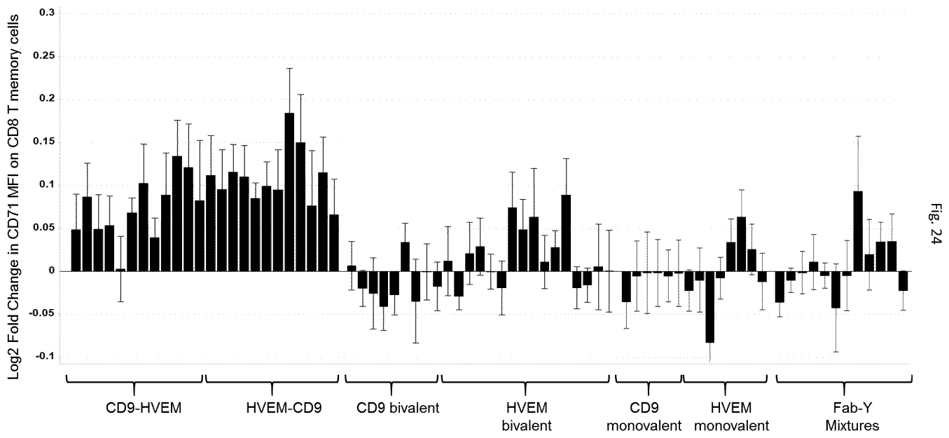

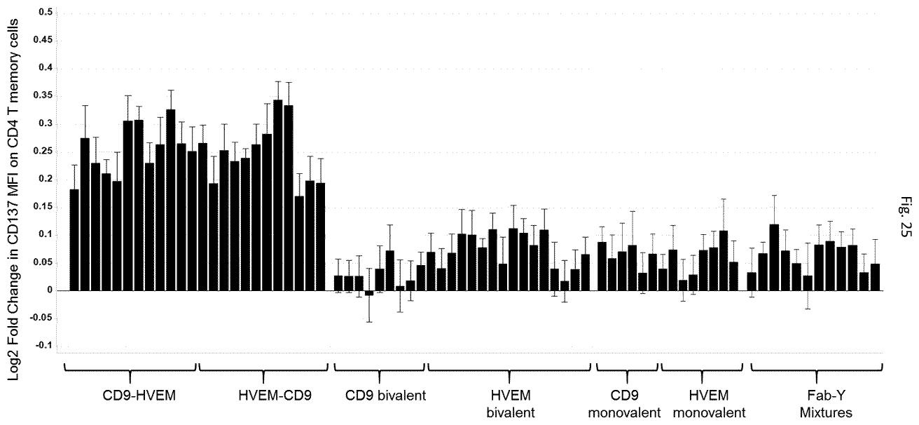

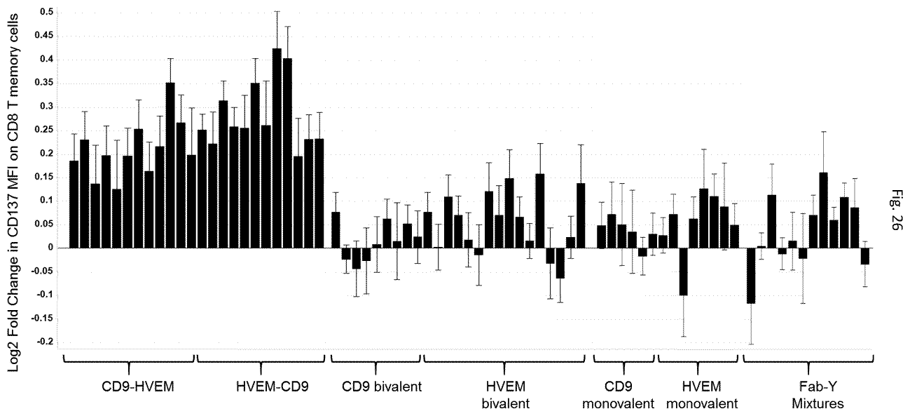

The levels of expression on CD4+ and CD8+ memory T cells of CD71 (FIGS. 23 and 24, respectively) and CD137 (FIGS. 25 and 26, respectively) was also investigated. The HVEM-CD9 bispecific antibodies showed an effect resulting in the increase of the level of expression of CD71 and CD137 markers, irrespective of the orientation of the Fab-X/Fab-Y bispecific antibody.

The bivalents (i.e. formed by a fusion where both Fab in the Fab-X and Fab-Y are specific for CD9 or HVEM as the case may be) and monovalent antibodies for CD9 or HVEM (i.e. formed by a fusion where the Fab is specific for CD9 or HVEM but the other component Fab is a negative control) did not lead to a similar increase in the activation marker fluorescence intensity on either cell populations, suggesting that a monospecific antibody binding CD9 alone or HVEM alone, whether in a monovalent or bivalent fashion, is unable to stimulate activation in the absence of the binding to the other antigen.

CD9-CD7 Bispecific Antibodies

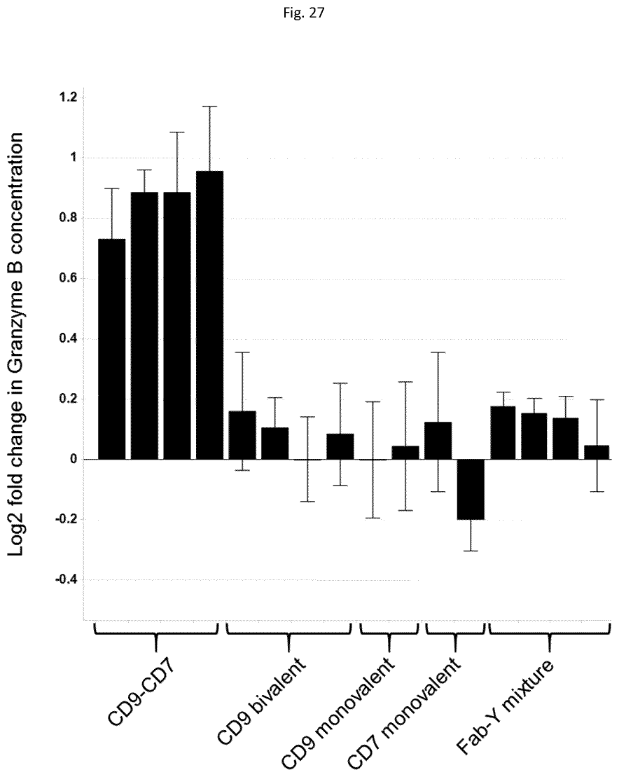

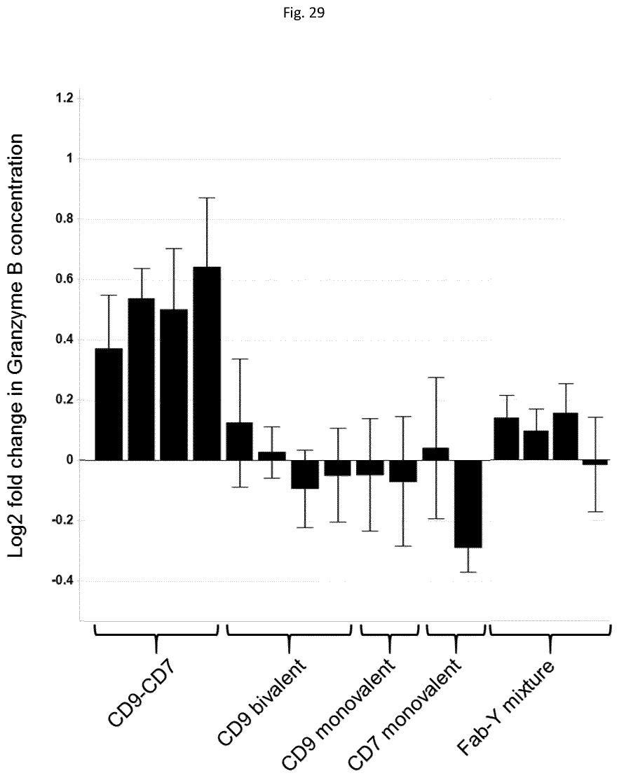

Four CD9-CD7 bispecific antibodies showed increased levels of secreted granzyme B under SEB (FIG. 27) and anti-CD3 stimulation (FIG. 29). The control constructs did not lead to this increase; the CD9 bivalents (i.e. formed by a fusion where both Fab in the Fab-X and Fab-Y are specific for CD9) and monovalent antibodies for CD9 or CD7 (i.e. formed by a fusion where the Fab is specific for CD9 or CD7 but the other component Fab is a negative control) did not lead to a similar increase in the secretion of granzyme B, suggesting that the binding of either CD9 or CD7 alone cannot induce granzyme B secretion in the absence of the other.

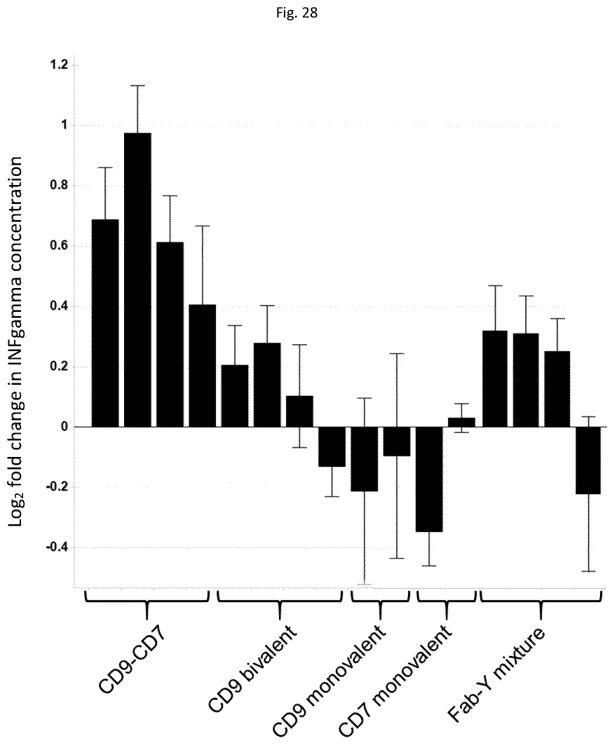

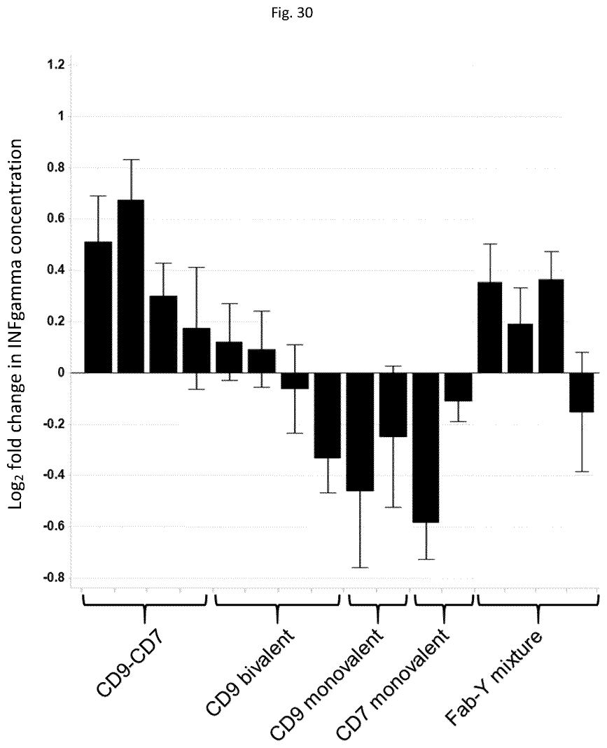

Similar increases in IFNgamma could be detected for the CD9-CD7 bispecific antibodies, although to a lesser degree that that seen for granzyme B (FIGS. 28, for SEB stimulation and FIG. 30 for the anti-CD3 stimulation). The response of the CD9 bivalent antibodies and monovalent controls was less clear also with a range of small increases and decreases in the level of secreted IFNgamma in either anti-CD3 or SEB stimulated conditions. However, there remained a clear enhancement of response, particularly in SEB stimulated conditions, of the CD7-CD9 bispecific antibodies as opposed to anti-CD7 and anti-CD9 antibodies as a mixture.

### Example 3: Comparison of Fab-X/Fab-Y Bispecific Antibodies with IgG Single Specificity Controls and Mixtures

To confirm the need for the antigen-binding portions binding CD9 and another antigen to be on the same antibody, in order to elicit the stimulatory effect, IgG1 antibodies were generated. These antibodies are monospecific and bivalent, which means they comprise each two antigen-binding portions for the same antigen, for example one antigen-binding portion binding CD9 and one antigen-binding portion binding CD137. The IgG antibodies were then assayed individually and as a mixture on 4 PBMC donors in anti-CD3 conditions and compared to the corresponding Fab-X/Fab-Y bispecific antibodies and controls.

A grid of fusion proteins Fab-X and Fab-Y were created by combining equimolar (1 μM) quantities of Fab-X (Fab-scFv) and Fab-Y (Fab-peptide) with specificity for CD9 and CD137 in TexMACS™ media (Miltenyi Biotec®) containing 100 U/mL penicillin/100 μg/mL streptomycin. Mixtures of equimolar (1 μM) Fab-Y proteins were also generated in the same manner. The IgG antibodies were diluted to 500 nM individually or as a mixture. The Fab-X and Fab-Y fusion proteins were incubated together for 1 hour (in a 37° C15% CO₂ environment), at a final concentration of 500 nM. Negative control wells contained TexMACS™ media only were also generated alongside the Fab-X and Fab-Y wells.

During this time, cryopreserved human PBMC isolated from platelet leukapheresis cones were thawed and washed in TexMACS™ media and resuspended at 3.33×10⁶ cells/ml. The PBMC were then seeded into 384-well flat bottom tissue culture plates (Greiner Bio-one®) at 30 μL/well (1×10⁵ PBMCs).

A total of 10 μL of CD9/CD137 bispecific antibodies or IgG antibodies were transferred to the plates containing 30 μL PBMCs. The PBMCs were then either left unstimulated by the addition of 10 μL of TexMACS media, or stimulated with 10 μL soluble anti-CD3 (UCHT1) (10 ng/mL final concentration). This resulted in a final assay concentration of Fab-X/Fab-Y bispecific antibodies of 100 nM. The plates were then returned to a 37° C./5% CO₂ environment for 48 hours.

After 48 hours the plates were centrifuged at 500×g for 5 minutes at 4° C. Cell culture conditioned media were transferred from the cell pellets to fresh plates and frozen at −80° C. Cells were washed with 60 μL FACS buffer two times by centrifugation, followed by resuspension of the pellets by shaking the plates at 2200 rpm for 30 seconds. The cells were stained with a cocktail of fluorescently labeled antibodies as listed in Table 1 and incubated at 4° C. in the dark for 1 hour. After this time, cells were washed twice with FACS buffer, before fixing with 2% paraformaldehyde (BD CellFix™, diluted in dH₂O) and stored overnight at 4° C. The plates were centrifuged at 500×g for 5 minutes, the fixation buffer aspirated to waste and the cells resuspended in a residual volume of 10 μL for acquisition on the iQue® Screener Plus (IntelliCyt®). The data analysis software package ForeCyt™ (IntelliCyt®) was used to gate on CD14+ monocytes, CD56+ NK cells, CD19+ B cells, CD4+ and CD8+ memory and naïve T cells. For each cell population the cellular expression of CD25 and CD71 was measured as reported median fluorescent intensity values. The data were then used to calculate the log 2 fold changes of expression relative to control well values.

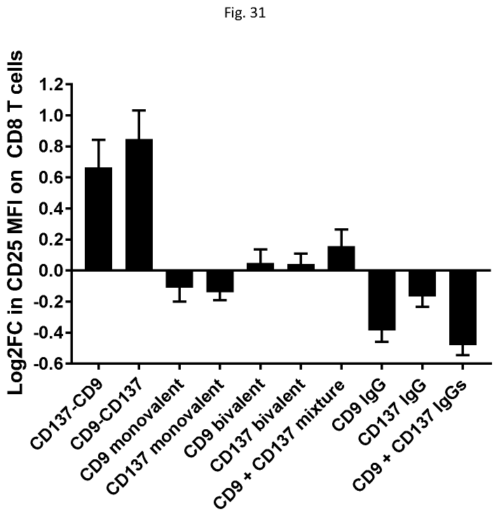

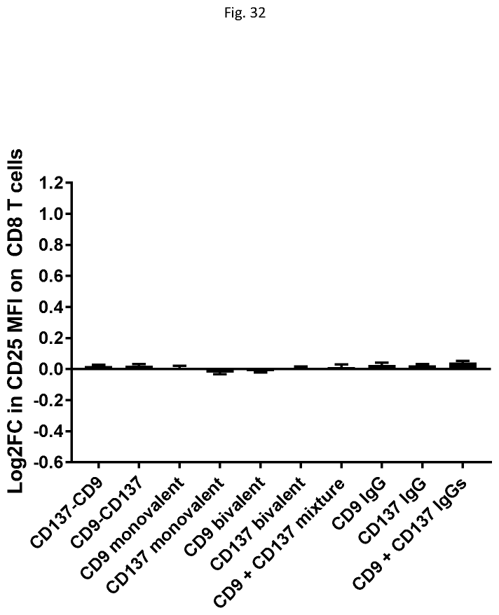

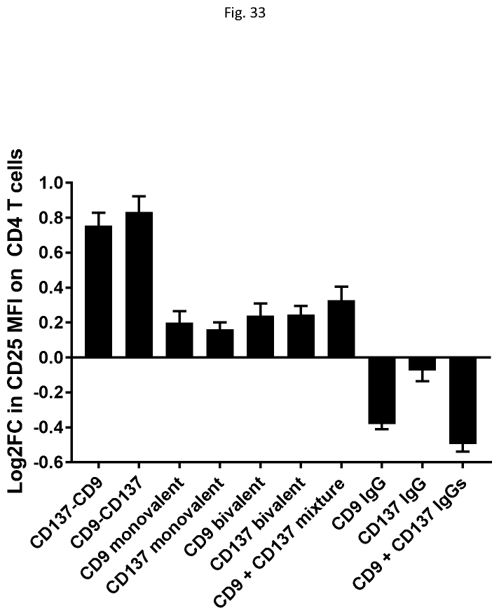

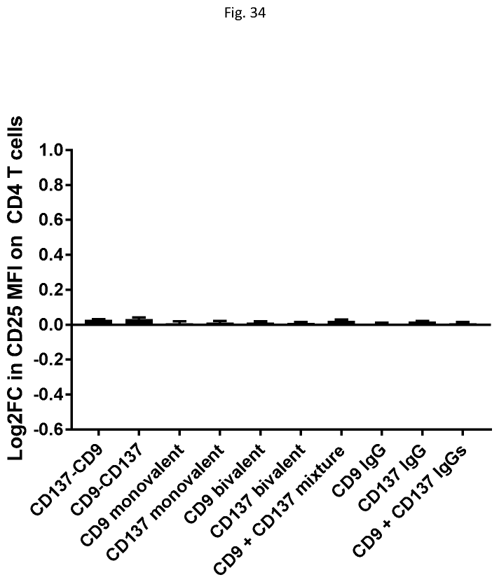

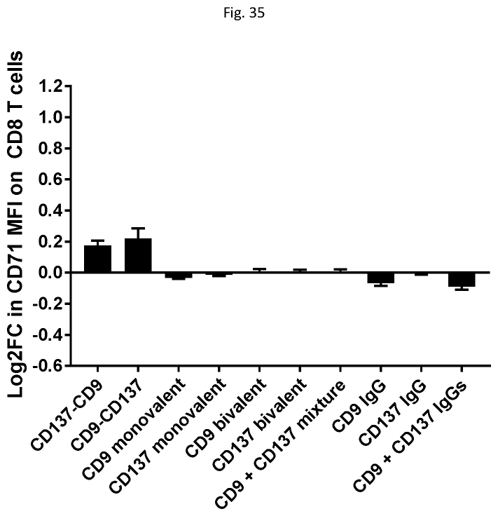

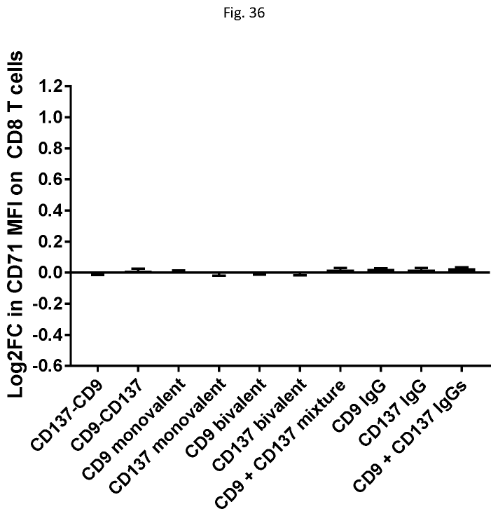

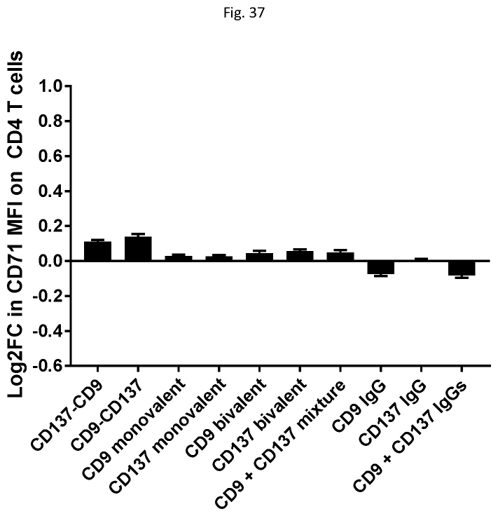

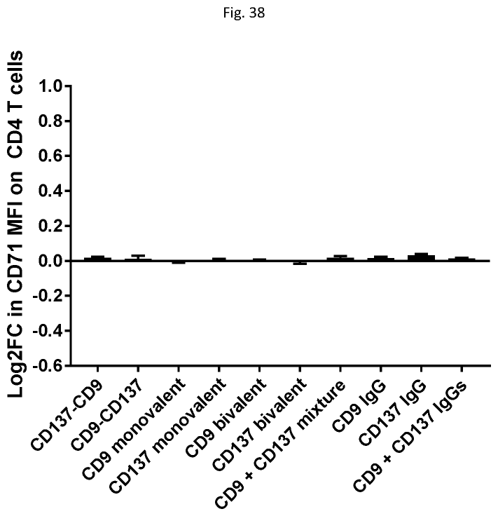

As previously shown the CD9/CD137 bispecific antibodies caused an increase in the level of CD25 on both CD4+ and CD8+ T cells when anti-CD3 stimulated conditions (FIGS. 31 and 33, first and second bars). On the contrary the monovalent and bivalent antibodies caused no change, irrespective on the orientation of the antigen-binding portions within the antibody. The anti-CD9 and anti-CD137 IgG antibodies, either individually or as a mixture, also did not lead to an increase in CD25 or CD71 expression on the T cell populations (FIGS. 31, 33, 35 and 37, last three bars). No effect could be seen by any antibody treatment in unstimulated conditions (FIGS. 32, 34, 36 and 38).

### Example 4: Effect of CD9 Bispecific Antibodies on T Cell Proliferation

The effect of CD9 bispecific antibodies on proliferation of CD4+ and CD8+ T cells was assessed in PBMC donors. Mixtures of fusion proteins Fab-X and Fab-Y were created by diluting equimolar (1 μM) quantities of Fab-X (Fab-scFv) and Fab-Y (Fab-peptide) with specificity for CD9 and CD137 in DMEM (ThermoFisher) containing 10% FBS and 100 U/mL penicillin/100 μg/mL streptomycin. Mixtures of equimolar (1 μM) Fab-Y proteins were also generated in the same manner. The Fab-X and Fab-Y fusion proteins were incubated together for 1 hour (in a 37° C15% CO₂ environment).

During this time, cryopreserved human PBMCs isolated from platelet leukapheresis cones were thawed, washed in DMEM media and resuspended at approximately 2×10⁶ cells/mL in PBS. Cell Trace™ Violet (CTV; ThermoFisher) was then added to a final concentration of 10 μM (2 μl 5 mM CTV in DMSO added to 10 mL cells), incubated in the dark at room temperature for 10 minutes, the cells washed twice with DMEM and resuspended in media to a final concentration of 1×10⁶ cells/mL.

Fab-X/Fab-Y bispecific antibodies were diluted to 400 nM concentration in DMEM and 50 transferred in to wells of a 96-well U bottom tissue culture plate. Anti-CD3 (clone UCHT1), 50 μL of a 40 ng/mL solution in DMEM, was then added. To 3 wells 100 μL DMEM media alone was added as a negative proliferation control. Finally, 100 μL of CTV labeled PBMC were added to each well. This resulted in a final assay concentration of Fab-X/Fab-Y bispecific antibodies of 100 nM, 10 ng/mL anti-CD3 and 1×10⁵ cells/well. The plates were then returned to a 37° C./5% CO₂ environment for 96 hours.

After 96 hours the plates were centrifuged at 500×g for 5 minutes. Cell culture conditioned media were removed, and the cells resuspended in 100 μL PBS containing fluorescently labeled antibodies as listed in Table 4. The cells were incubated at room temperature in the dark for 15 min, washed with PBS, resuspended in 100 μL/per well PBS and analysed by flow cytometry (BD FACS Canto II™). Total events from 50 μL (50% volume) of each well were collected.

The data analysis software package FlowJo® was used to gate on CD8+ and CD4+ T cells. Cell proliferation was assessed by enumerating cells with reduced CTV staining relative to unstimulated cells. The data are presented as the mean±SEM for triplicate wells.

TABLE 4

| Epitope | Fluorophore | Clone | Source | Dilution |
|---|---|---|---|---|
| CD8 | Pacific blue | HIT8a | BioLegend ® | 200 |
| CD4 | APC | RPA-T4 | BioLeqend ® | 20 |

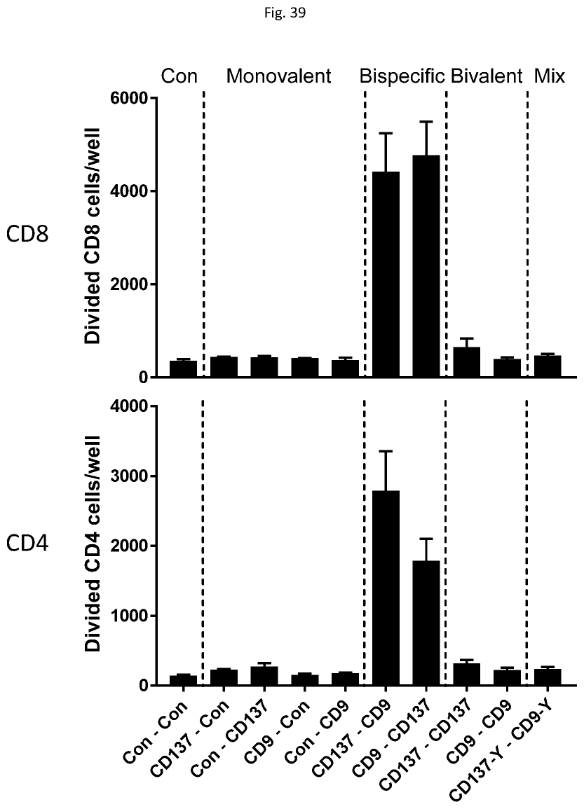

The bispecific anti-CD137/CD9 antibodies and anti-CD9/CD137 antibody (both orientations exemplified) were shown to increase the proliferation of both CD4+ and CD8+ T cells when added to PBMCs stimulated with soluble anti-CD3 for 96 hours. The bivalents antibodies formed by Fab-X and Fab-Y antibodies where both Fabs are specific for either CD137 or CD9 (as the case may be) and monovalent antibodies for CD137 and CD9, formed by fusions where the Fab in the Fab-X is specific for CD137 or CD9 but the Fab in the Fab-Y is a negative control (in the present case the Fab of an anti-idiotypic antibody, hence not for an antigen expressed on or by any cell) did not lead to a similar increase in proliferation in the absence of the other. Furthermore, the mixture of Fab-Y binding CD9 and Fab-Y binding CD137 also had no stimulatory effect on the T cell populations, This further confirms that there is a requirement, for the stimulatory function to occur, for the antigen-binding portions binding CD9 and CD137 to be on the same molecule, or on separate molecules which become associated via non-covalent or covalent associations or linkers (FIG. 39).

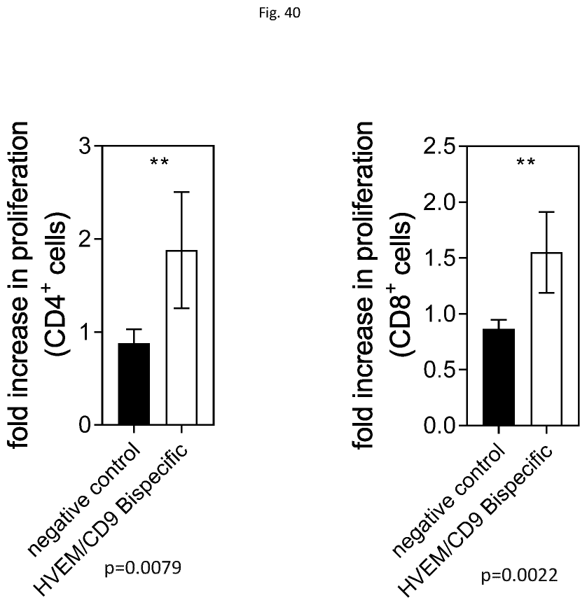

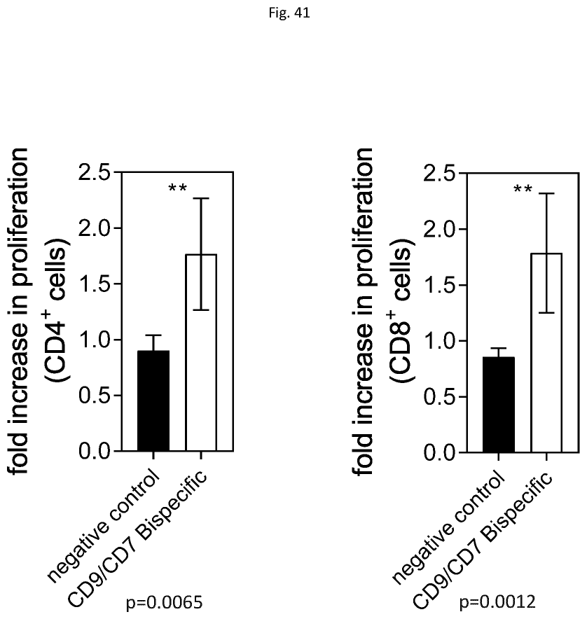

Similarly, proliferation of CD8+ and CD4+ T cells in the presence of anti-CD3 (50 ng/mL) stimulation was statistically significantly higher when human PBMC were treated with CD9-HVEM (FIG. 40) or CD9-CD7 (FIG. 41) bispecific antibodies versus the negative control (i.e. no bispecific antibody).

The significance of this difference between treatment with control and bispecific antibody is expressed as a p value calculated using Mann-Whitney test.

### Example 5: Bispecific Antibodies Concentration-Response Studies

The CD9/CD137 bispecific antibody was titrated using PBMC from 2 donors.

Mixtures of fusion proteins Fab-X and Fab-Y were created by diluting equimolar (1 μM) quantities of Fab-X (Fab-scFv) and Fab-Y (Fab-peptide) with specificity for CD9, CD137 or non-binding control as indicated in DMEM (ThermoFisher) containing 10% FBS and 100 U/mL penicillin/100 μg/mL streptomycin then incubated together for 1 hour (in a 37° C./5% CO₂ environment).

During this time, cryopreserved human PBMC isolated from platelet leukapheresis cones were thawed, washed in DMEM media and resuspended at approx. 2×10⁶ cells/mL in PBS. Cell Trace™ Violet (CTV; ThermoFisher) was then added to a final concentration of 10 μM (2 μl 5 mM CTV in DMSO added to 10 mL cells), incubated in the dark at room temperature for 10 minutes, the cells washed twice with DMEM and resuspended in media to a final concentration of 1×10⁶ cells/mL.

Fab-X/Fab-Y bispecific antibodies were diluted to 400 nM concentration in DMEM then 1:10 and 1:100 dilutions of this made also in DMEM. Aliquots of 50 μL were then transferred to wells of a 96-well U bottom tissue culture plate. Anti-CD3 (clone UCHT1), 50 μL of a 200 ng/mL solution in DMEM, was then added. To 3 wells 100 μL DMEM media alone was added as a negative proliferation control. Finally, 100 μL of CTV labeled PBMC was added to each well. This resulted in a final assay concentration of Fab-X and Fab-Y complexes of 100 nM, 10 nM or 1 nM plus 10 ng/mL anti-CD3 and 1×10⁵ cells/well. The plates were then returned to a 37° C./5% CO₂ environment for 96 hours.

After 96 hours the plates were centrifuged at 500×g for 5 minutes. Cell culture conditioned media were removed, and the cells resuspended in 100 μL PBS containing fluorescently labeled antibodies as listed in Table 3 above. The cells were incubated at room temperature in the dark for 15 minutes, washed with PBS, resuspended in 100 μL/per well PBS and analysed by flow cytometry (BD FACS Canto II). Total events from 50 μL (50% volume) of each well were collected.

The data analysis software package FlowJo® was used to gate on CD4+ and CD8+ T cells. Cell proliferation was assessed by enumerating cells with reduced CTV staining relative to unstimulated cells. The data are presented as the mean±SEM for triplicate wells.

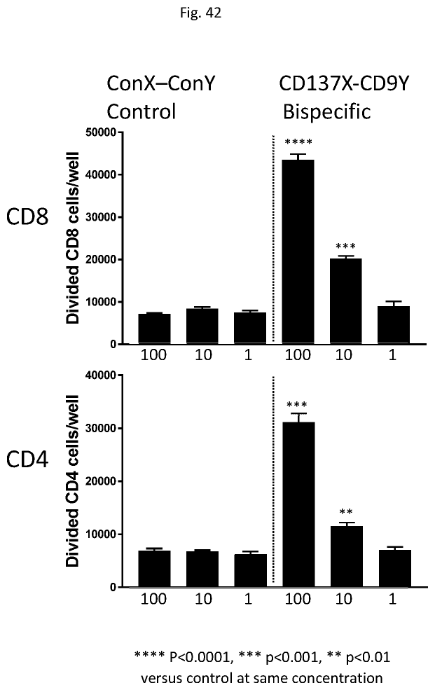

As shown in FIG. 42, an anti-CD137-CD9 bispecific antibody was capable to enhance T cell proliferation and such effect could be titrated down over three logs of decreasing antibody concentrations. The significance of this effect is reflected in the p values between samples treated with the bispecific antibodies and control at each concentration. P values were calculated using an unpaired students' t test in Graphpad Prism®.

### Example 6: Comparison of the Effects of CD9 Bispecific Antibodies to Nivolumab and Ipilumumab

Ipilimumab (an anti-CTLA-4 antibody) was the first checkpoint inhibitor to be approved in 2011 as a treatment for melanoma, closely followed by FDA approval of anti-PD1 directed antibodies, pembrolizumab and nivolumab in 2014 (Hargadon et al., International Immunopharmacol. 62:29-39 (2018)). Whilst there are still significant challenges in understanding differences in efficacy across patient groups, ranging from complete responses, to treatment relapse and even failure to respond, (Haslam and Prasad. JAMA Network Open.5:2e192535 (2019)), these molecules represent current clinically-validated references for immunotherapy in a range of cancer types and have been utilised in the present studies for benchmarking the activity of the novel bispecific antibodies described herein.

The effect of an anti-CD137-CD9 bispecific antibody on the level of CD71 and proliferation of CD4+ and CD8+ T cells was assessed in 4 PBMC donors and compared to equimolar quantities of ipilimumab (Yervoy®) and nivolumab (Opdivo®) as clinically tested reference molecules.

A grid of fusion proteins Fab-X and Fab-Y were created by diluting equimolar (1 μM) quantities of Fab-X (Fab-scFv) and Fab-Y (Fab-peptide) with specificity for CD9 and CD137 in TexMACS™ media (Miltenyi Biotec®) containing 100 U/mL penicillin/100 μg/mL streptomycin and 5% human AB Serum (Sigma-Aldrich). The Fab-X and Fab-Y fusion proteins were incubated together for 1 hour (in a 37° C., 5% CO₂ environment), at a final concentration of 500 nM. Negative control wells containing TexMACS™ media only were also generated alongside the Fab-X and Fab-Y wells. ipilimumab and nivolumab were diluted to 500 nM in TexMACS™ media containing 100 U/mL penicillin/100 μg/mL streptomycin and 5% human AB Serum (Sigma-Aldrich).

During this time, cryopreserved human PBMC isolated from platelet leukapheresis cones were thawed and washed in TexMACS™ media (containing 100 U/mL penicillin/100 μg/mL streptomycin and 5% human AB Serum (Sigma-Aldrich)) and resuspended in 10 mL PBS (ThermoFisher). 10 μl of 5 mM CellTrace™ Violet solution was added to the sample and mixed well by inversion. The cells were incubated at 37° C. for 20 minutes and washed by the addition of 45 mL PBS containing 10% heat inactivated foetal bovine serum (ThermoFisher Scientific). The cells were incubated for a further 5 minutes before centrifugation at 400×g for 5 minutes. The waste was removed, and the cells resuspended at 2.5×10⁶ cells/mL. The PBMC were then seeded into 96-well U-bottom tissue culture plates (Costa®) at 60 μl/well (1.5×10⁵ PBMC). A total of 20 μl of Fab-X/Fab-Y bispecific antibodies were transferred to the plates containing 60 μL PBMC. The PBMC were then either left unstimulated by the addition of 20 μl of media or stimulated with 20 μl of soluble anti-CD3 (clone UCHT-1) (50 ng/mL final concentration) with and without 200 ng/mL anti-CD28 (clone CD28.2). This resulted in a final assay concentration of antibodies of 100 nM. The plates were then returned to a 37° C., 5% CO₂ environment for 6 days.

After 6 days the plates were centrifuged at 500×g for 5 minutes at 4° C. Cell culture conditioned media was transferred from the cell pellets to fresh plates and frozen at ⁻80° C. Cells were washed with 60 μl FACS buffer by centrifugation, followed by resuspension of the pellets by shaking the plates at 1800 rpm for 30 seconds. The cells were stained with a cocktail of fluorescently labeled antibodies as listed in Table 5 and incubated at 4° C. in the dark for 30 minutes. After this time, cells were washed with FACS buffer and fixed with 2% paraformaldehyde for one hour at 4° C. before the addition of 150 μl FACS buffer. The plates were centrifuged at 500×g for 5 minutes, the fixation buffer aspirated to waste and the cells resuspended in a residual volume of 15 μl for acquisition on the iQue® Screener Plus (IntelliCyt®).

The data analysis software package ForeCyt™ (IntelliCyt®) was used to exclude CD14⁺ monocytes, followed by the identification of the CD4⁺ and CD8⁺ T cells. For each cell population the cellular expression of CD71 was measured as reported median fluorescent intensity values. The level of CellTrace™ Violet was also measured to identify cells having undergone cell division. The data were then used to calculate the log 2 fold changes in CD71 expression and the number of divided CD4 and CD8 positive T cells relative to the control wells.

TABLE 5

| Epitope | Fluorophore | Clone | Source | Dilution |
|---|---|---|---|---|
| CD14 | PE/Cy7 | M5E2 | BD Pharmagen ® | 80 |
| CD8 | BV605 | RPA-T8 | BioLegend ® | 40 |
| CD4 | FITC | RPA-T4 | BioLegend ® | 80 |
| CD71 | PE | M-A712 | BioLegend ® | 160 |
| Live/Dead | Near IR | | Thermo Scientific ® | 1000 |

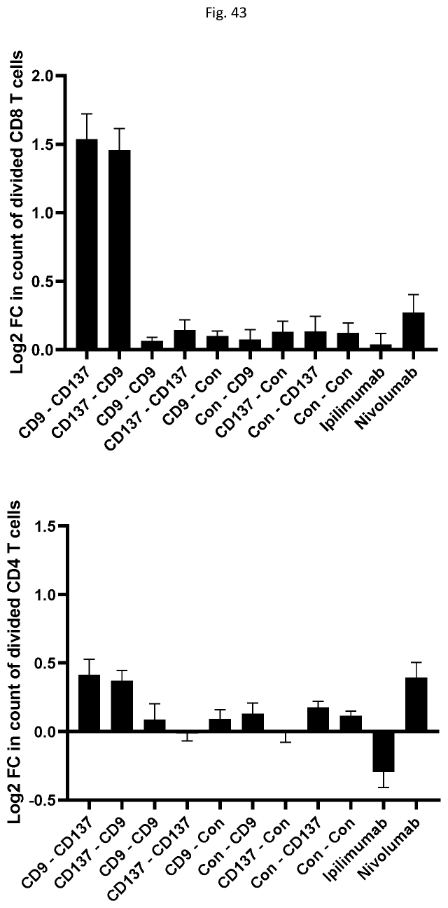

CD137-CD9 bispecific Fab-K_{D}-Fab antibodies led to increases in the number of dividing CD8⁺ T cells when PBMC cultures were treated in combination with 50 ng/mL anti-CD3 (UCHT-1) for 6 days. This increase was less apparent in the CD4⁺ T cells of these cultures, however small increases could be detected (FIG. 43). Bivalent and monovalent controls did not lead to any increase in the number of dividing CD4⁺ or CD8⁺ T cells. Ipilimumab did not have any impact of the number of dividing T cells, while Nivolumab did lead to small increases, but to a much smaller degree than that observed for the bispecific antibody on CD8⁺ T cells.

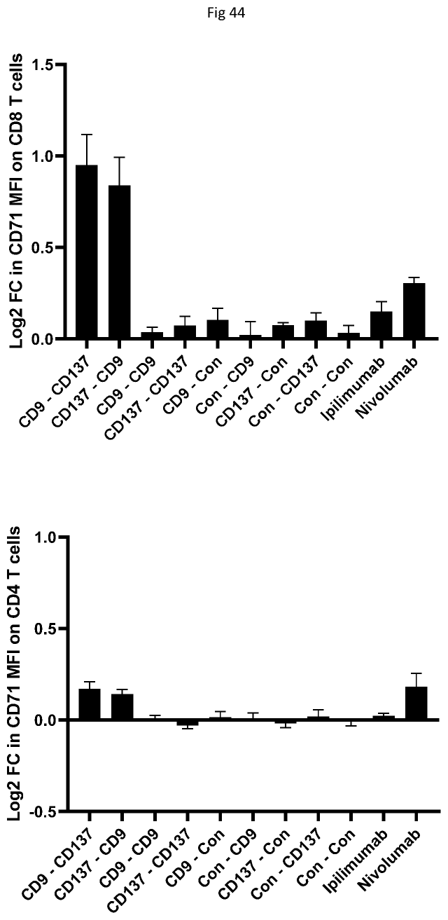

The log 2 fold change in the CD71 MFI of the activation marker on CD8⁺ and CD4⁺ T cells showed a very similar result (FIG. 44), supporting the finding that the CD137-CD9 bispecific antibody increased the activation of CD8⁺ T cells, in particular when in combination with a sub-maximal anti-CD3 stimulus, while the bivalent and monovalent controls did not.

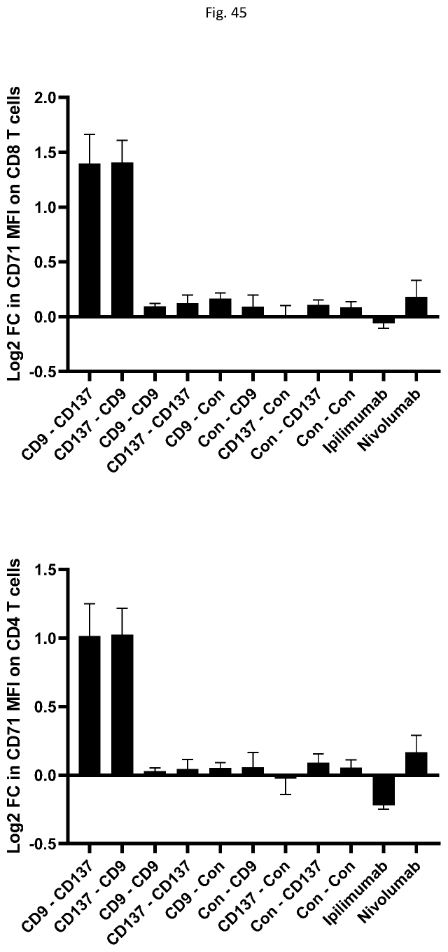

These constructs were also tested in combination with a sub-maximal anti-CD3 stimulus in combination with of anti-CD28 co-stimulation (clone 28.2). Increases in CD71 MFI on both CD8⁺ and CD4⁺ T cells could be detected (FIG. 45), while no increase was measured in response to the bivalent and monovalent controls or commercial comparators.

The activity of ipilimumab and nivolumab was compared to another CD9 bispecific antibody, a CD9-CD7 bispecific antibody.

A grid of fusion proteins Fab-X and Fab-Y were created by diluting equimolar (1 μM) quantities of Fab-X (Fab-scFv) and Fab-Y (Fab-peptide) with specificity for CD9 and CD7 in TexMACS™ media (Miltenyi Biotec®) containing 100 U/mL penicillin/100 μg/mL streptomycin. The Fab-X and Fab-Y fusion proteins were incubated together for 1 hour (in a 37° C., 5% CO₂ environment), at a final concentration of 500 nM IgG CD9-CD7 bispecific antibodies (generated using established methodologies in the art), ipilimumab (Yervoy®) and nivolumab (Opdivo®) were diluted to 500 nM in TexMACS™ media containing 100 U/mL penicillin/100 μg/mL streptomycin. Negative control wells containing TexMACS™ media only were also generated alongside the Fab-X and Fab-Y wells.

During this time, cryopreserved human PBMC isolated from platelet leukapheresis cones were thawed and washed in TexMACS™ media and resuspended at 2.5×10⁶ cells/mL. The PBMC were then seeded into 96-well U-bottom tissue culture plates (Costar) at 60 μL/well (1.5×10⁵ PBMC).

20 μL of Fab-X and Fab-Y complexes were transferred to the plates containing 60 μL PBMC. The PBMC were then either left unstimulated by the addition of 20 μL of TexMACS™ media or stimulated with 20 μL of SEB (1 μg/ml final concentration). This resulted in a final assay concentration of antibody treatment of 100 nM. The plates were then returned to a 37° C., 5% CO₂ environment for 48 hours.

After 48 hours the plates were centrifuged at 500×g for 5 minutes at 4° C. Conditioned media was transferred from the cell pellets to fresh plates and frozen at −80° C. On the day of analysis, the conditioned media were thawed and diluted 50-fold in RPMI and assayed for levels of granzyme B using an IntelliCyt® QBead® PlexScreen.

The data analysis software package ForeCyt™ (IntelliCyt®) was used to measure the median fluorescent intensity values for the granzyme B detection beads. The log 2 fold changes of granzyme B concentrations were calculated relative to control well values.

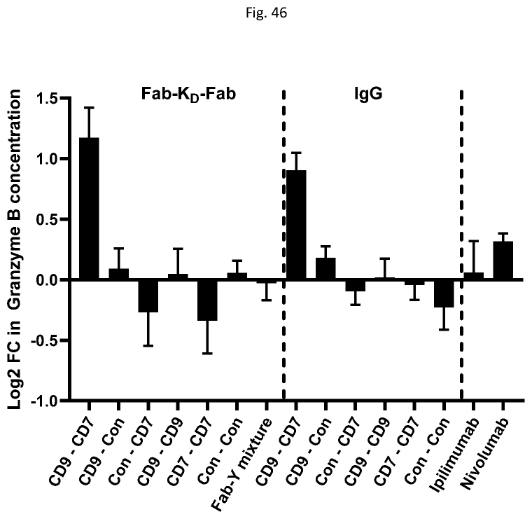

As reported previously the CD9-CD7 bispecific Fab-K_{D}-Fab constructs led to an increase in the level of granzyme B in the conditioned medium of PBMC cultures after stimulation with SEB for 48 hours (FIG. 46). There was no similar increase in granzyme B detection when samples were treated with the bivalent or monovalent controls. Furthermore, a mixture of the anti-CD9 and CD7 Fab-Y constructs did not increase granzyme B levels in the supernatant. The IgG CD9-CD7 bispecific antibody gave similar increases in granzyme B as the Fab-K_{D}-Fab suggesting successful conversion to a potential therapeutic format. Furthermore, the bivalent and monovalent IgG controls did not increase granzyme B levels in the conditioned medium.

Neither ipilimumab nor nivolumab had an effect on the level of granzyme B in this assay, showing the superiority of an anti-CD9-CD7 bispecific antibody over the current CPIs therapies.

### Example 7: Effect of a CD9 Bispecific Antibody on NK Cell Activation and Degranulation

Natural killer (NK) cells are a subset of lymphocytes that play a central role in the innate immune response to tumours and viral infections. They kill by a mechanism termed “degranulation” that involves the release of cytolytic granules containing granzyme B and perforin. NK cells are key effectors in cancer immunosurveillance and have many different mechanisms to distinguish targets cells from healthy cells based on complex balance in expression of activating and inhibitory receptors. The tumour microenvironment exploits these mechanisms to inhibit NK activity and different strategies are being explored to try and enhance their activity and/or prevent their suppression by the tumour microenvironment for cancer immunotherapy (Guillery, C. et al. Nat. Immunol. 17 (9) 1025-1036. 2016).

During degranulation, cytolytic granules in NK cells are released and the lysosome-associated membrane protein-1 (LAMP-1, CD107a) which is present on cytolytic granules surface is transported to the cell surface and becomes measurable as a biomarker of NK cell degranulation activity. This results in the expression of lysosomal proteins such as CD107a on the cell surface which can be used as a sensitive marker of cytotoxic degranulation.

The effect of an anti-CD9 bispecific antibody, an anti-CD9/CD7 bispecific antibody, on NK cell activation and degranulation was assessed in 3 PBMC donors. Mixtures of fusion proteins Fab-X and Fab-Y were created by diluting equimolar (400 nM) quantities of Fab-X (Fab-scFv) and Fab-Y (Fab-peptide) with specificity for CD7 and/or CD9 in RPMI (ThermoFisher) containing 10% FBS and 2 mM GlutaMAX. Mixtures of equimolar (400 nM) Fab-Y proteins were also generated in the same manner. The Fab-X and Fab-Y fusion proteins were incubated together for 1 hour in a 37° C./5% CO₂ incubator. Following this incubation 50 μL of each antibody was transferred in quadruplicate to wells of a 96 well U bottom tissue culture plate.

During this time, PBMC were isolated from fresh whole blood using centrifugation and were washed twice in PBS. The cells were resuspended at 5×10⁶ cells/mL in RPMI media and 100 μL was transferred into the assay plate containing the antibodies. The plate was placed in a 37° C./5% CO₂ incubator for 30 minutes.

After 30 minutes K562 target cells were resuspended at 1×10⁶ cells/mL in RPMI and 50 μL was added to the plate containing PBMC and antibodies. This equates to an effector to target ratio (E:T) of 10:1. The final assay concentration of Fab-X+Fab-Y complexes equates to 100 nM. The assay plate was then returned to the 37° C./5% CO₂ incubator for 2 hours. PBMC cultured alone, with no target cells, were used as a negative control for degranulation.

After 2 hours the plate was centrifuged at 300×g for 3 minutes. Conditioned medium was removed, the cells were washed twice with cell staining buffer (Biolegend) and then resuspended in 100 μL cell staining buffer containing fluorescently labeled antibodies, as listed below in Table 6. The cells were incubated at 4° C. in the dark for 20 minutes, washed twice with cell staining buffer, resuspended in 150 μL/per well PBS and analysed by flow cytometry (BD FACS Canto II™). From each well 100 μL of each sample was collected.

The data analysis software package FlowJo® was used to gate on CD3-CD56+ NK cells. Degranulation and activation of NK cells was assessed by analysing appearance of cell surface CD107a or CD69 geometric mean fluorescence. The percentage increase in CD107a+ cells or percentage increase in CD69 was calculated compared to levels observed in PBMC and K562 co-cultures without any antibodies. The data from the three donors was pooled and presented as both individual donors (black circles) and the mean±SEM (horizontal line).

TABLE 6

| Epitope | Fluorophore | Clone | Source | Dilution |
|---|---|---|---|---|
| CD3 | FITC | UCHT1 | Biolegend | 100 |
| CD56 | Brilliant Violet 421 ™ | HCD56 | Biolegend | 100 |
| CD107a | APC | H4A3 | Biolegend | 100 |
| CD69 | PerCP | FN50 | Biolegend | 100 |

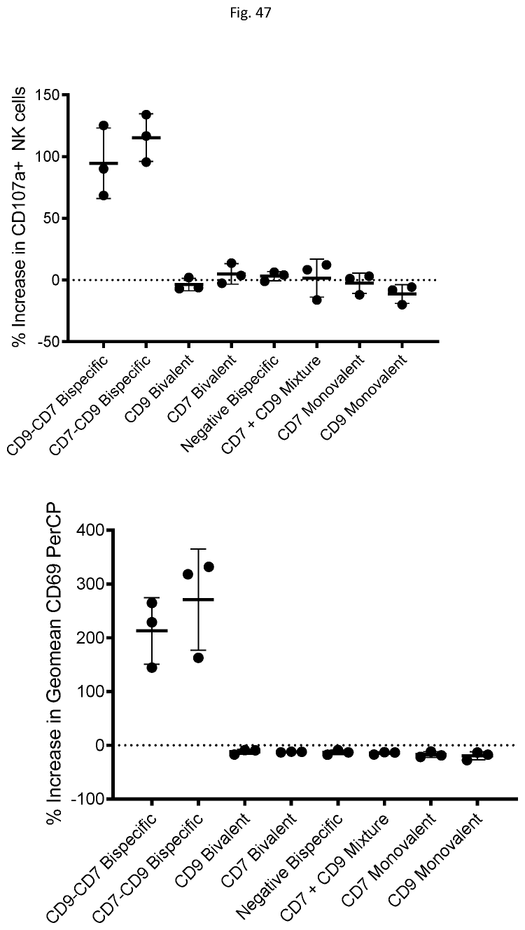

As shown in FIG. 47 degranulation of NK cells is greatly increased following treatment with CD7-CD9 bispecific antibodies. The increase in CD107a+ NK cells is only observed when the two antigens are targeted with the bispecific CD7-CD9, as bivalent and monovalent controls do not change the level of degranulation. This increase in degranulation following treatment with CD7-CD9 bispecific antibodies is matched by an increase in NK cells activation as shown by an increase in CD69 expression. These results suggest that an anti-CD7-CD9 bispecific antibody according to the present invention may promote NK cells activity in the tumour microenvironment for cancer immunotherapy.

### Example 8: Evaluation of the Effect of an Anti-CD9 Bispecific Antibody on Peptide Stimulated Melanoma Patient PBMC

Peripheral blood mononuclear cells (PBMC) from patients with melanoma were isolated on Ficoll-Paque® PLUS by density centrifugation. PBMC were cultured at 2×10⁵ cells/well and stimulated with a peptide pool comprising sequences from known melanoma tumour-associated antigens (PepMix™ collection Melanoma PM-CMel, JPT Innovative Peptide Solutions). Cells were cultured in the absence (1% PBS vehicle control), or presence of test antibodies. Purified Fab-X and Fab-Y against CD9 and CD137 were incubated together for 60 minutes (in a 37° C15% CO₂ environment) at an equimolar concentration. The final concentration of both Fab-K_{D}-Fab complex and IgG molecules was 100 nM. Where sufficient cells were available, a melanoma peptide-stimulated vehicle control (1% PBS) condition was also included. Cells were cultured in RPMI 1640 medium (with sodium bicarbonate and L-glutamine, Sigma-Aldrich) with 100 U/mL penicillin/100 μg/mL streptomycin (Sigma-Aldrich) and 5% (v/v) heat-inactivated human AB serum (Sigma-Aldrich) in 96-well round bottom plates (Sarstedt) for 6 days at 37° C./5% CO₂ in 95% air.

Following 6 days in culture, plates were centrifuged, and conditioned medium removed and stored at −20° C. prior to cytokine analysis. The remaining cells were stained with anti-human CD3-BV510, CD4-PeCy7, CD8-FITC, CD56-BV421, CD25-APC and CD71-PE and Zombie NIR™ fixable viability dye (BioLegend), and then fixed using FoxP3 staining buffer set (BD Biosciences). Following fixation, cells were washed and resuspended in buffer (PBS plus 5% foetal bovine serum). Results were analysed using FlowJo™ version 10. Absolute concentrations of IFNγ, TNFα, IL-2 and IL-10 in the conditioned medium were measured by Luminex® (Biotechne/R&D Systems) according to manufacturer's instructions. Samples were diluted 1:2 and analysed using a Bio-Plex® 200 reader and Bio-Plex® Manager™ software.

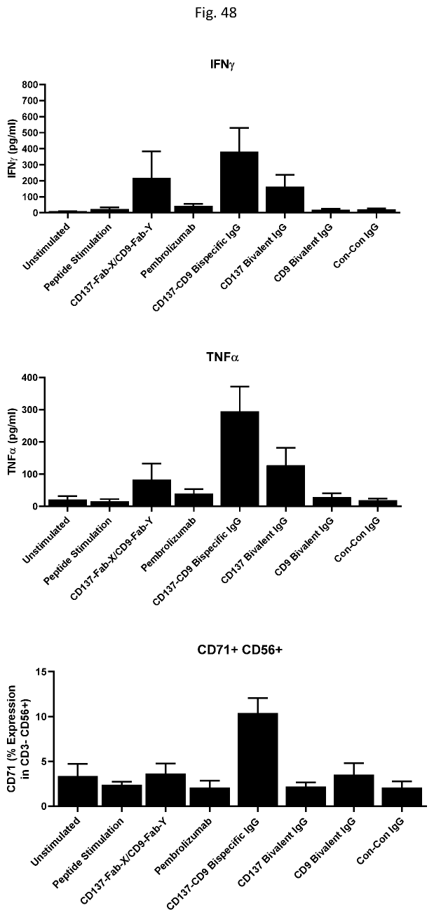

FIG. 48 summarises the effects of all antibodies and treatments on the release of IFNγ, TNFα and NK cell activation from peptide-stimulated melanoma PBMC from patients. The melanoma antigen peptide mix failed to stimulate increased levels of IFNγ, TNFα or effect NK cell activation in these samples, reflecting an immunosuppressed phenotype characteristic of previous observations in melanoma. However, both the CD137-CD9 Fab-K_{D}-Fab complex and CD137-CD9 IgG were able to stimulate elevated release of both IFNγ and TNFα in all donors tested. The CD137 bivalent showed similar effects to the CD137-CD9 Fab-K_{D}-Fab with respect to the effect on IFNγ and TNFα release, but this was of lower magnitude than the effect of the CD137-CD9 bispecific IgG. With respect to elevated CD71 expression as a marker of NK cell activation, the CD137-CD9 bispecific IgG, was the only treatment that was capable of enhancing NK cell activation. Under these experimental conditions, pembrolizumab (an anti-PD1 inhibitory antibody) which blocks the PD-1 mediated down-regulation of T cell and myeloid cell activation in the tumour microenvironment, did not exhibit any stimulatory activity with respect to either cytokine release or NK cell activation, clearly evidencing the superior and advantageous effect of a bispecific antibody against CD137 and CD9.

### Example 9: Evaluation of the Effect of an Anti-CD9 Bispecific Antibody in a Colorectal Tumour Micro-Environment Model

The BioMAP® Colorectal Cancer (CRC) panel (Eurofins) models the interactions between the immune-stromal (fibroblasts) and immune-vascular (endothelial cells) environments in the context of colorectal cancer (HT-29 colorectal adenocarcinoma cell line). The interactions between tumour cells, stimulated peripheral blood mononuclear cells (PBMC), and the host stromal network are modeled in the StroHT29 system, whilst the VascHT29 system captures the interactions between tumour cells, activated PBMC and vascular tissue. The biomarkers selected for the BioMAP® CRC panel reflect a range of activities related to inflammation, immune-function, tissue remodeling and metastasis, modeling tumour-mediated immune suppression that occurs in the tumour microenvironment (TME) of cancer patients. The StroHT29 system is comprised of the HT-29 colorectal adenocarcinoma cell line, human neonatal dermal fibroblasts (HDFn) and PBMC. The VascHT29 system is comprised of the HT-29 colorectal adenocarcinoma cell line, human umbilical vein endothelial cells (HUVEC) and PBMC. Both systems are stimulated by sub-mitogenic levels of SEB and the stimulation conditions are optimised to activate or prime T cells, but not drive T cell proliferation.

Human primary cells used in the BioMAP® systems were at early passage (passage 4 or earlier) to minimise adaptation to cell culture conditions and preserve physiological signaling responses. Primary cells were commercially purchased, pooled from multiple donors (n=3-6), and handled according to manufacturer's instructions. The HT-29 colorectal adenocarcinoma cell line was purchased from American Type Culture Collection (ATCC).

Adherent cell types were cultured in 96-well plates until confluent, followed by the addition of PBMC. Test antibodies were prepared in PBS and added at 100 nM, 1 hour prior to stimulation for 48 hours. Each plate also contained several controls, including negative unstimulated controls vehicle controls and drug reference controls.

Direct ELISA was used to measure biomarker levels of cell-associated and cell membrane targets. Soluble factors from conditioned medias are quantified using either HTRF® detection, multiplex electrochemiluminescence assay or capture ELISA. Effects of test agents on cell viability (cytotoxicity) were measured by sulforhodamine B (SRB) for adherent cells (48 hours), and by AlamarBlue® reduction for PBMC (42 hours). All test agents were tested at 100 nM in triplicate. Data acceptance criteria were based on plate performance (% CV of controls <10%) and the performance of controls across assays with a comparison to historical controls.

Biomarker measurements were profiled in triplicate for antibody-treated samples, and then averaged and divided by the average of vehicle control samples (at least 6 vehicle controls from the same plate) to generate a fold change that is then log 2 transformed. Statistical p values were calculated from unpaired t-test statistics of raw data values compared to vehicle controls. Significant changes in biomarkers are defined as changes induced by the test or reference molecules, that have an effect size >20% compared to the vehicle control (log 2 ratio >0.2) and a p value <0.01.

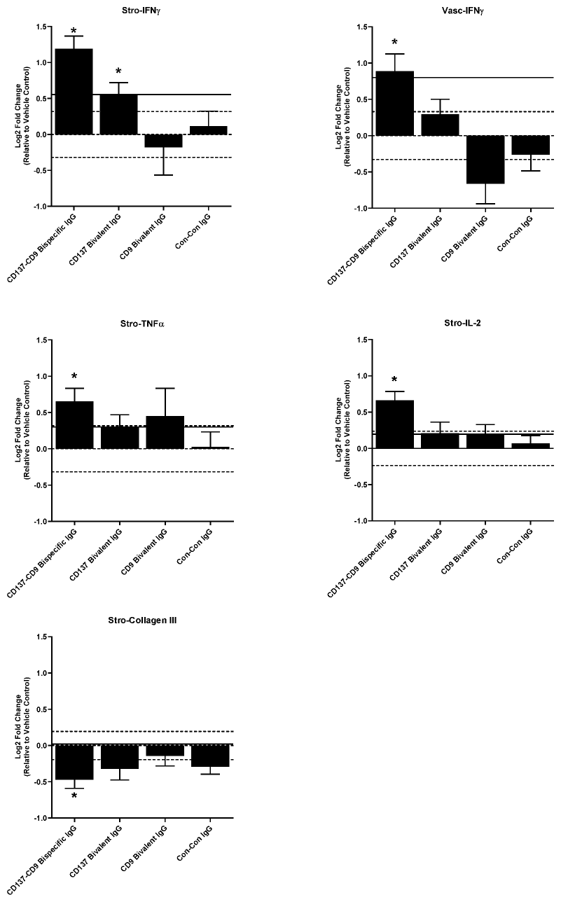

CD137-CD9 bispecific IgG was not cytotoxic to any cell type in either the StroHT29 or VascHT29 systems. CD137-CD9 bispecific IgG was associated with increased inflammation and immune-related activities, including increases in IFNγ, IL-2 and TNFα in the StroHT29 system indicating an immune restorative capacity consistent with the profile of approved anti-PD-1 antibodies, such as pembrolizumab which was used as a reference molecule in this assay (FIG. 49). Additionally, CD137-CD9 bispecific IgG also decreased collagen III, possibly indicating a potential matrix-inhibitory/anti-fibrotic effect that could allow increased immune infiltration into the TME. The CD137 bivalent IgG (similar to urelumab anti-CD137 IgG), inhibited IFNγ production in the StroHT29 system, but this effect was approximately 50% of that observed with the CD137-CD9 bispecific molecule, suggesting a greatly enhanced bispecific-mediated effect. Conversely, the CD9 bivalent IgG showed no indication of immune restoration via induction of pro-inflammatory cytokines such as IFNγ in the StroHT29 system, but interestingly did inhibit IFNγ production in the VascHT29 system.

### Example 10: Identification of the Binding Specificity of CD9 Bispecific Antibodies

Seven different CD9 antibodies each providing unique functional activity with CD137, CD7 and HVEM antibodies in a bispecific combination were analysed for their ability to bind the long extracellular loop 2 of CD9. CD9 contains two extracellular loops: a short extracellular loop (loop 1: 34-55 in SEQ ID NO:1) and a long extracellular loop (loop 2: 112-195 in SEQ ID NO:1). We used biolayer interferometry to assess binding of our panel of CD9 Fab-Y antibodies (selected for ability to bind full-length CD9 expressed on cells) to the long extracellular loop 2 peptide, using the Octet® RED384 System (FortéBio) with anti-hIgG Fc Capture (AHC) Biosensors (FortéBio) at room temperature. Firstly, an array of 8 sensors was dipped in kinetics buffer (PBS 0.1%, BSA 0.02%, Tween 20) for 120 seconds to provide a baseline signal. Sensors were then moved to wells containing 200 μl of recombinant human CD9 long extracellular loop 2-human Fc fusion protein (10015-CD, R&D Systems®) at 2 μg/mL in kinetics buffer to immobilize the protein to the biosensor (100 seconds), followed by a second baseline step (180 seconds) in kinetics buffer to equilibrate the biosensors now coated with the CD9 long extracellular loop 2-human Fc fusion protein. No detachment of the peptide was observed during this step. Sensors were then dipped in wells containing one of the anti-CD9 Fab (10 μg/mL) to evaluate association of each antibody to CD9-loop2 (120 seconds), followed by dissociation in kinetics buffer (600 seconds). Anti-CD137 Fab-Y (11175) was used as negative control during the antibody association step. A new set of 8 biosensors was used to repeat this process for each of the anti-CD9 antibodies (7270, 7271, 7272, 7485,7486, 7489, 7491).

As shown in Table 8, all the CD9 antibodies are functional when combined as a bispecific antibody with anti-CD137, anti-CD7 and anti-HVEM.

TABLE 8

| | CD9 | | | |
|---|---|---|---|---|
| | extracellular | Functional | Functional | Functional |
| CD9 | loop 2 | with CD137 | with CD7 | with HVEM |
| antibody | binding | (Y/N) | (Y/N) | (Y/N) |
| 7270 | ✓ | Y | Y | Y |
| 7271 | ✓ | Y | Y | Y |
| 7272 | ✓ | Y | Y | Y |
| 7485 | ✓ | Y | Y | Y |
| 7486 | ✓ | Y | Y | Y |
| 7489 | ✓ | Y | Y | Y |
| 7491 | ✓ | Y | Y | Y |

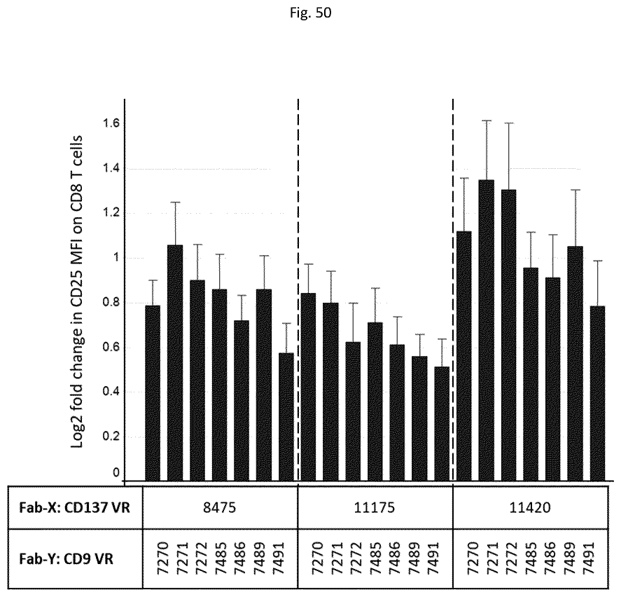

A positive functional response for CD137-CD9 is considered to be the capacity to increase CD25 expression on T cells by greater than 0.5 log 2 fold change in 3 donors when added as a Fab-K_{D}-Fab with anti-CD137 antibodies 8475, 11175 and 11420 as shown in FIG. 50.

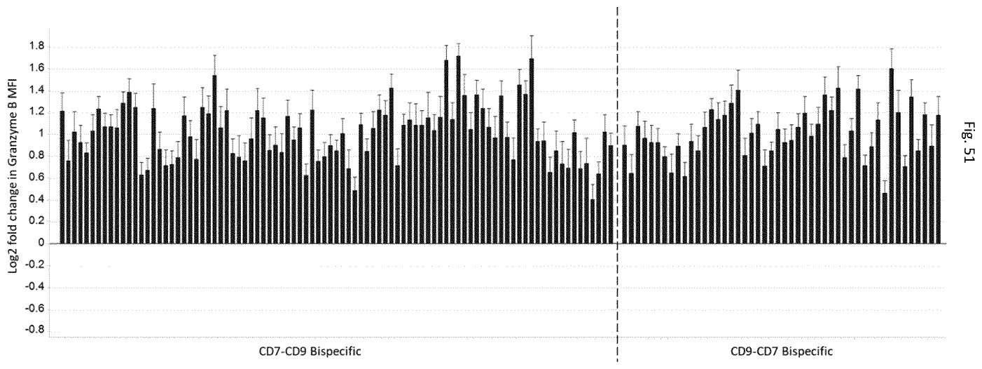

A positive functional response for CD7-CD9 is considered to be the capacity to increase granzyme B greater than 0.5 log 2 fold change MFI in 3 donors when added as a Fab-K_{D}-Fab with 14 different anti-CD7 antibodies as shown in FIG. 51.

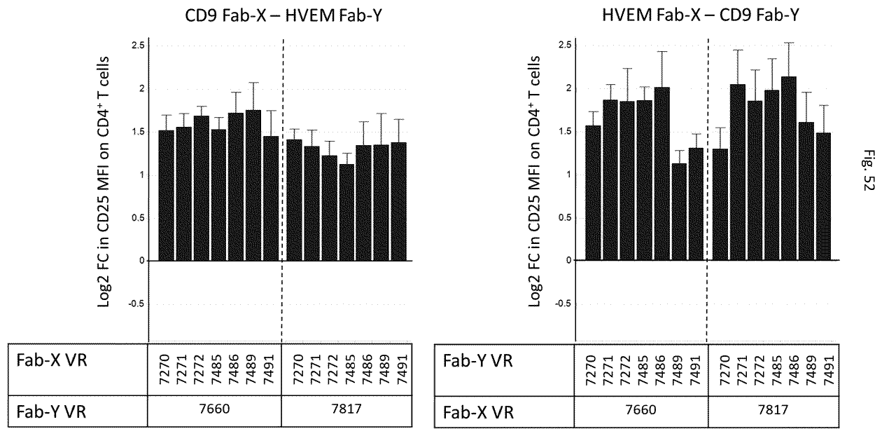

A positive functional response for HVEM-CD9 is considered to be the capacity to increase CD25 expression on T cells by greater than 0.5 log 2 fold change in 2 donors when added as a Fab-K_{D}-Fab with anti-HVEM antibodies 7660 and 7817 as shown in FIG. 52.

1. An antibody which comprises at least a first antigen-binding portion binding CD9 and a second antigen-binding portion binding another antigen.

2. The antibody according to claim 1, wherein the second antigen-binding portion binds an antigen expressed on a cell surface, such as a cell of the immune system.

3. The antibody according to claim 2, wherein the cell is a T-cell, a B-cell or a NK cell.

4. The antibody according to any one of claims 1 to 3, wherein the second antigen-binding portion binds CD137.

5. The antibody according to any one of claims 1 to 3, wherein the second antigen-binding portion binds HVEM.

6. The antibody according to any one of claims 1 to 3, wherein the second antigen-binding portion binds CD7.

7. The antibody according to any one of the preceding claims, wherein each of the antigen-binding portions is a monoclonal antigen-binding portion.

8. The antibody according to any one of the preceding claims, wherein each of the antigen-binding portions is independently selected from a Fab, a Fab′, a scFv or a VHH.

9. The antibody according to any one of the preceding claims, wherein the antigen-binding portions are the antigen-binding portions of an IgG.

10. The antibody according to any one of the preceding claims wherein the antibody is chimeric, human or humanised, preferably the antibody is humanised.

11. The antibody according to any one of the preceding claims wherein the antibody comprises a heavy chain constant region selected from an IgG1, an IgG2, IgG3 or an IgG4 isotype, or a variant thereof.

12. The antibody according to anyone of the preceding claims, wherein the antibody further comprises at least one additional antigen-binding portion.

13. The antibody according to claim 12, wherein the additional antigen-binding portion is capable of increasing the half-life of the antibody.

14. The antibody according to claim 13, wherein the additional antigen-binding portion binds albumin, preferably human serum albumin.

15. The antibody according to any one of the preceding claims wherein the first antigen binding portion binds CD9 in CD9 loop 2, wherein preferably the first antigen-binding portion binds within amino acids 112 to 195 of SEQ ID NO: 1.

16. The antibody according to any one of claims 1 to 4 or 7 to 15 wherein the first antigen-binding portion binding CD9 comprises a first heavy chain variable region and a first light chain variable region and the second antigen-binding portion comprises a second heavy chain variable region and a second light chain variable region and binds CD137 and wherein:
a. The first heavy chain variable region comprises a CDR-H1 comprising SEQ ID NO: 11, a CDR-H2 comprising SEQ ID NO: 12 and a CDR-H3 comprising SEQ ID NO: 13; and
b. The first light chain variable region comprises a CDR-L1 comprising SEQ ID NO: 14, a CDR-L2 comprising SEQ ID NO: 15 and a CDR-L3 comprising SEQ ID NO: 16; and
c. The second heavy chain variable region comprises a CDR-H1 comprising SEQ ID NO: 5, a CDR-H2 comprising SEQ ID NO: 6 and a CDR-H3 comprising SEQ ID NO: 7; and
d. The second light chain variable region comprises a CDR-L1 comprising SEQ ID NO: 8, a CDR-L2 comprising SEQ ID NO: 9 and a CDR-L3 comprising SEQ ID NO: 10;
or
e. The first heavy chain variable region comprises SEQ ID NO: 21 and the first light chain variable region comprises SEQ ID NO: 23; and the second heavy chain variable region comprises SEQ ID NO: 17 and second light chain variable region comprises SEQ ID NO: 19;
or
f. The first heavy chain variable region is encoded by a nucleotide sequence comprising SEQ ID NO: 22 and the first light chain variable region is encoded by a nucleotide sequence comprising SEQ ID NO: 24; and the second heavy chain variable region is encoded by a nucleotide sequence comprising SEQ ID NO: 18 and second light chain variable region is encoded by a nucleotide sequence comprising SEQ ID NO: 20.

17. The antibody according to any one of claims 1 to 3 or 5 or 7 to 15 wherein the first antigen-binding portion binding CD9 comprises a first heavy chain variable region and a first light chain variable region and the second antigen-binding portion comprises a second heavy chain variable region and a second light chain variable region and binds HVEM and wherein:
a. The first heavy chain variable region comprises a CDR-H1 comprising SEQ ID NO: 11, a CDR-H2 comprising SEQ ID NO: 12 and a CDR-H3 comprising SEQ ID NO: 13; and
b. The first light chain variable region comprises a CDR-L1 comprising SEQ ID NO: 14, a CDR-L2 comprising SEQ ID NO: 15 and a CDR-L3 comprising SEQ ID NO: 16; and
c. The second heavy chain variable region comprises a CDR-H1 comprising SEQ ID NO: 25, a CDR-H2 comprising SEQ ID NO: 26 and a CDR-H3 comprising SEQ ID NO: 27; and
d. The second light chain variable region comprises a CDR-L1 comprising SEQ ID NO: 30;
or
e. The first heavy chain variable region comprises SEQ ID NO: 21 and the first light chain variable region comprises SEQ ID NO: 23; and the second heavy chain variable region comprises SEQ ID NO: 31 and second light chain variable region comprises SEQ ID NO: 33;
or
f. The first heavy chain variable region is encoded by a nucleotide sequence comprising SEQ ID NO: 22 and the first light chain variable region is encoded by a nucleotide sequence comprising SEQ ID NO: 24; and the second heavy chain variable region is encoded by a nucleotide sequence comprising SEQ ID NO: 32 and second light chain variable region is encoded by a nucleotide sequence comprising SEQ ID NO: 34.

18. The antibody according to any one of claims 1 to 3 or 6 to 15 wherein the first antigen-binding portion binding CD9 comprises a first heavy chain variable region and a first light chain variable region and the second antigen-binding portion comprises a second heavy chain variable region and a second light chain variable region and binds CD7 and wherein:
a. The first heavy chain variable region comprises a CDR-H1 comprising SEQ ID NO: 11, a CDR-H2 comprising SEQ ID NO: 12 and a CDR-H3 comprising SEQ ID NO: 13; and
b. The first light chain variable region comprises a CDR-L1 comprising SEQ ID NO: 14, a CDR-L2 comprising SEQ ID NO: 15 and a CDR-L3 comprising SEQ ID NO: 16; and
c. The second heavy chain variable region comprises a CDR-H1 comprising SEQ ID NO: 35, a CDR-H2 comprising SEQ ID NO: 36 and a CDR-H3 comprising SEQ ID NO: 37; and
d. The second light chain variable region comprises a CDR-L1 comprising SEQ ID NO: 38, a CDR-L2 comprising SEQ ID NO: 39 and a CDR-L3 comprising SEQ ID NO: 40;
or
e. The first heavy chain variable region comprises SEQ ID NO: 21 and the first light chain variable region comprises SEQ ID NO: 23; and the second heavy chain variable region comprises SEQ ID NO: 41 and second light chain variable region comprises SEQ ID NO: 43;
or
f. The first heavy chain variable region is encoded by a nucleotide sequence comprising SEQ ID NO: 22 and the first light chain variable region is encoded by a nucleotide sequence comprising SEQ ID NO: 24; and the second heavy chain variable region is encoded by a nucleotide sequence comprising SEQ ID NO: 42 and second light chain variable region is encoded by a nucleotide sequence comprising SEQ ID NO: 44.

19. A pharmaceutical composition comprising the antibody according to any one of the preceding claims and one or more pharmaceutically acceptable excipients.

20. The antibody according to any one of claims 1 to 18 or the pharmaceutical composition according to claim 19 for use in therapy.

21. The antibody according to any one of claims 1 to 18 or the pharmaceutical composition according to claim 19 for use in the treatment of cancer and/or an infectious disease.

22. The antibody for use according to claim 21, wherein the antibody or the composition are for use concomitantly or sequentially to one or more additional cancer therapies.

23. A method for treating a subject afflicted with cancer and/or an infectious disease, comprising administering to the subject a pharmaceutically effective amount of an antibody according to any one of claims 1 to 18 or a pharmaceutical composition according to claim 19.

24. The method according to claim 23, wherein the antibody or the composition are administered concomitantly or sequentially to one or more additional cancer therapies.

25. Use of an antibody according to any one of claims 1 to 18 or a pharmaceutical composition according to claim 19 in the manufacture of a medicament for treating cancer.

26. The use according to claim 25, wherein the antibody or the composition are for use concomitantly or sequentially to one or more additional cancer therapies.


SEQUENCE LISTING (44 sequences)

SEQ ID NO: 1 - protein, 228 aa, Homo sapiens
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 2 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 3 - protein, 283 aa, Homo sapiens
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 4 - protein, 240 aa, Homo sapiens
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 5 - protein, 10 aa, Artificial Sequence
  CDR-H1 VR8475
GFSLSTYAMS

SEQ ID NO: 6 - protein, 16 aa, Artificial Sequence
  CDR-H2 VR8475
AIWSGGTTDYTSWAKG

SEQ ID NO: 7 - protein, 12 aa, Artificial Sequence
  CDR-H3 VR8475
MAVIFNAYTFDS

SEQ ID NO: 8 - protein, 11 aa, Artificial Sequence
  CDR-L1 VR8475
QASQNIYSYLA

SEQ ID NO: 9 - protein, 7 aa, Artificial Sequence
  CDR-L2 VR8475
GASTLAS

SEQ ID NO: 10 - protein, 13 aa, Artificial Sequence
  CDR-L3 VR8475
QQGHSSGNVGNNV

SEQ ID NO: 11 - protein, 10 aa, Artificial Sequence
  CDR-H1 VR7272
GFSLSSYAMG

SEQ ID NO: 12 - protein, 16 aa, Artificial Sequence
  CDR-H2 VR7272
AIGSITATGYARWAKG

SEQ ID NO: 13 - protein, 12 aa, Artificial Sequence
  CDR-H3 VR7272
EIYVGSAYAFDI

SEQ ID NO: 14 - protein, 11 aa, Artificial Sequence
  CDR-L1 VR7272
QASQSISNYLA

SEQ ID NO: 15 - protein, 7 aa, Artificial Sequence
  CDR-L2 VR7272
LASTLAS

SEQ ID NO: 16 - protein, 11 aa, Artificial Sequence
  CDR-L3 VR7272
QQGYIDNVNKG

SEQ ID NO: 17 - protein, 117 aa, Artificial Sequence
  VH VR8475
QSVEESGGRLVTPGTPLTLTCTVSGFSLSTYAMSWVRQAPGKGLEWIGAIWSGGTTDYTSWAKGRFTISKASTTVDLKITSPTTEDTATYFCVRMAVIFNAYTFDSWGPGTLVTVSS

SEQ ID NO: 18 - DNA, 424 bp, Artificial Sequence
  VH nucl VR8475
aagcttcgaagccaccatggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcagtacgtatgcaatgagctgggtccgccaggctccagggaaggggctggagtggatcggagcgatttggagtggtggtaccacggactacacgagctgggcgaaaggccgattcaccatctccaaagcctcgaccacggtggatctgaaaatcaccagtccgacaaccgaggacacggccacctatttctgtgtcagaatggcagtgatttttaatgcttacacttttgattcctggggcccaggcaccctggtcaccgtctcgagt

SEQ ID NO: 19 - protein, 111 aa, Artificial Sequence
  VL VR8475
AYDMTQTPSSVSAAVGGTVTIKCQASQNIYSYLAWYQQKPGQRPKLLIYGASTLASGVPSRFKGSGSGTDFTLTISDLECDDAATYYCQQGHSSGNVGNNVFGGGTEVVVK

SEQ ID NO: 20 - DNA, 421 bp, Artificial Sequence
  VL nucl VR8475
aagcttcgaagccaccatgaacatgagggcccccactcagctgctggggctcctgctgctctggctcccaggtgccagatgtgcctatgatatgacccagactccatcctccgtgtctgcagctgtgggaggcacagtcaccatcaagtgccaggccagtcagaacatttacagttacttagcctggtatcagcagaaaccagggcagcgtcccaagctcctgatctatggtgcgtccactctggcatctggggtcccatcgcggttcaaaggcagtggatctgggacagatttcactctcaccatcagcgacctggagtgtgacgatgctgccacttactactgtcaacagggccatagtagtggtaatgttggaaataatgttttcggcggagggaccgaggtggtggtcaaacgtacg

SEQ ID NO: 21 - protein, 117 aa, Artificial Sequence
  VH VR7272
QSLEESGGRLVTPGTPLTLTCTVSGFSLSSYAMGWVRQAPGKGLEWIGAIGSITATGYARWAKGRFSISKTSTTVDLKMTSPTTEDTATYFCAREIYVGSAYAFDIWGPGTLVTVSS

SEQ ID NO: 22 - DNA, 424 bp, Artificial Sequence
  VH nucl VR7272
aagcttcgaagccaccatggagactgggctgcgctggcttctcctggtcgctgtgctcaaaggtgtccagtgtcagtcgctggaggagtccgggggtcgcctggtcacgcctgggacacccctgacactcacctgcacagtctctggattctccctcagtagctatgcaatgggctgggtccgccaggctccagggaaggggctggagtggatcggagccattggtagtattactgccactggctacgcgcgctgggcaaaaggccgattcagcatctccaagacctcgaccacggtggatctgaaaatgaccagtccgacaaccgaggacacggccacctatttctgtgccagagagatttatgttgggtctgcttatgcctttgacatctggggcccaggcaccctggtcaccgtctcgagt

SEQ ID NO: 23 - protein, 109 aa, Artificial Sequence
  VL VR7272
AYDMTQTPASVEVAVGDTVTIKCQASQSISNYLAWYQQKPGQPPKLLIYLASTLASGVPSRFKGSGSGTEFTLTISDLECADAATYYCQQGYIDNVNKGFGGGTEVVVK

SEQ ID NO: 24 - DNA, 415 bp, Artificial Sequence
  VL nucl VR7272
aagcttcgaagccaccatggacacgagggcccccactcagctgctggggctcctgctgctctggctcccaggtgccagatgtgcctatgatatgacccagactccagcctctgtggaggtagctgtgggagacactgtcaccatcaagtgtcaggccagtcagagcattagtaactacttagcctggtatcagcagaaaccagggcagcctcccaagctcctgatctatctggcatctactctggcatctggggtcccatcgcggttcaaaggcagtggatctgggacagagttcactctcaccatcagcgacctggagtgtgccgatgctgccacttactattgtcaacagggttatattgataatgttaataaaggtttcggcggagggaccgaggtggtggtcaaacgtacg

SEQ ID NO: 25 - protein, 10 aa, Artificial Sequence
  CDR-H1 VR7660
GFSLSTYDMI

SEQ ID NO: 26 - protein, 16 aa, Artificial Sequence
  CDR-H2 VR7660
VITKTTTTYYANWAKG

SEQ ID NO: 27 - protein, 12 aa, Artificial Sequence
  CDR-H3 VR7660
DADDAVYYYFDI

SEQ ID NO: 28 - protein, 11 aa, Artificial Sequence
  CDR-L1
QASQSIGSGLA

SEQ ID NO: 29 - protein, 7 aa, Artificial Sequence
  CDR-L2 VR7660
AASTLTS

SEQ ID NO: 30 - protein, 12 aa, Artificial Sequence
  CDR-L3 VR7660
QEGVSRGNIDNA

SEQ ID NO: 31 - protein, 117 aa, Artificial Sequence
  VH VR7660
QSVEESGGRLVTPGTPLTLTCTVAGFSLSTYDMIWVRQAPGKGLEYIGVITKTTTTYYANWAKGRFTISKTSTTVDLKMTSPTTEDTATYFCARDADDAVYYYFDIWGPGTLVTVSS

SEQ ID NO: 32 - DNA, 423 bp, Artificial Sequence
  VH nucl VR7660
aagcttcgaagccaccatggagactgggctgcgctggcttctcctggtcgctgtgctcaaaggtgtccagtgtcagtcggtggaggagtccgggggtcgcctggtcacgcctgggacacccctgacactcacctgcacggtcgctggattctccctcagtacctacgacatgatctgggtccgccaggctccagggaaggggctggaatacatcggagtcattactaaaactactaccacatactacgcgaactgggcgaaaggccgattcaccatctccaaaacctcgaccacggtggatctgaaaatgaccagtccgacaaccgaggacacggccacctatttctgtgccagagatgctgatgatgctgtttattactactttgacatctggggcccaggcaccctggtcaccgtctcgag

SEQ ID NO: 33 - protein, 110 aa, Artificial Sequence
  VL VR7660
AYDMTQTPASAEVAVGGTVTINCQASQSIGSGLAWYQQKPGQRPKLLIYAASTLTSGVSSRFKGSGSGTEFTLTISGVECADAATYYCQEGVSRGNIDNAFGGGTEVVVK

SEQ ID NO: 34 - DNA, 418 bp, Artificial Sequence
  VL nucl VR7660
aagcttcgaagccaccatggacatgagggcccccactcagctgctggggctcctgctgctctggctcccaggtgccagatgtgcctatgatatgacccagactccagcctctgcggaggtagctgtgggaggcacagtcaccatcaattgccaggccagtcagagcattggcagtggtttggcctggtatcagcagaaaccagggcagcgtcccaaactcctgatctatgctgcatccactctgacatctggggtctcatcgcggttcaaaggcagtggatctgggacagagttcactctcaccatcagcggcgtggagtgtgccgatgctgccacttactactgtcaagagggtgttagtcgtggtaatattgataatgccttcggcggagggaccgaggtggtggtcaaacgtacg

SEQ ID NO: 35 - protein, 10 aa, Artificial Sequence
  CDR-H1 VR8850
GFSLSSFAMC

SEQ ID NO: 36 - protein, 16 aa, Artificial Sequence
  CDR-H2 VR8850
IINTGGSAYYASWATG

SEQ ID NO: 37 - protein, 14 aa, Artificial Sequence
  CDR-H3 VR8850
GNGYAGYGYDGFDP

SEQ ID NO: 38 - protein, 11 aa, Artificial Sequence
  CDR-L1 VR8850
QASQSITSWLS

SEQ ID NO: 39 - protein, 7 aa, Artificial Sequence
  CDR-L2 VR8850
AASKLTF

SEQ ID NO: 40 - protein, 12 aa, Artificial Sequence
  CDR-L3 VR8850
QSNYGSSSAYGA

SEQ ID NO: 41 - protein, 119 aa, Artificial Sequence
  VH VR8850
QSVEESGGRLVTPGTPLTLTCTVSGFSLSSFAMCWVRQAPGKGLEYIGIINTGGSAYYASWATGRFTISKTSTTVDLKISSPTTEDTATYFCARGNGYAGYGYDGFDPWGPGTLVTVSS

SEQ ID NO: 42 - DNA, 430 bp, Artificial Sequence
  VH nucl VR8850
aagcttcgaagccaccatggagactgggctgcgctggcttctcctggtcgctgtgctcaaaggtgtccagtgtcagtcggtggaggagtccgggggtcgcctggtcacgccggggacacccctgacactcacctgcacagtctctggattctccctcagtagctttgcaatgtgctgggtccgccaggctccagggaagggactggaatacatcggaatcattaatactggtggtagcgcatactacgcgagctgggcgacaggccgattcaccatctccaaaacctcgaccacggtggatctgaaaatctccagtccgacaaccgaggacacggccacctatttctgtgccagaggaaatggttatgctggttatggttatgatggttttgatccctggggcccaggcaccctggtcaccgtctcgagt

SEQ ID NO: 43 - protein, 110 aa, Artificial Sequence
  VL VR8850
DIVMTQTPASVSEPVGGTVTIKCQASQSITSWLSWYQQKPGQPPKLLIYAASKLTFGVSSRFRGSGSGTEYTLTISDLECADAATYYCQSNYGSSSAYGAFGGGTEVVVK

SEQ ID NO: 44 - DNA, 421 bp, Artificial Sequence
  VL nucl VR8850
aagcttcgaagccaccatggacacgagggcccccactcagctgctggggctcctgctgctctggctcccaggtgccagatgtgctgacattgtgatgacccagactccagcctccgtgtctgaacctgtgggaggcacagtcaccatcaagtgccaggccagtcagagcattaccagttggttatcctggtatcagcagaaaccagggcagcctcccaagctcctgatctacgcggcatccaaactgacatttggggtctcatcaagattcagaggcagtggatctgggacagagtacactctcaccatcagcgacctggagtgtgccgatgctgccacttactactgtcaaagcaattatggtagtagtagtgcttatggggctttcggcggagggaccgaggtggtggtcaaacgtacg